SEQUENCE LISTING (22 sequences)

SEQ ID NO: 1 - DNA, 1746 bp, Homo sapiens
atgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggtctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgggctggccattgccgccattgtaattggcattgtcgccctggcctgctccctggctgcctgcgtcggctgttgctgctgcaagaagaggagccaagctgtcctgatgcagatgaaggcacccaatgagtgttaa

SEQ ID NO: 2 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 3 - DNA, 2340 bp, Artificial
  LRRC15-Tev-Fc fusion coding region
atgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggtctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgagaacctgtacttccagggcgctagccaggagcccaaaagctctgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 779 aa, Artificial
  LRRC15-Tev-Fc fusion amino acid sequence
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSENLYFQGASQEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - RNA, 5938 nt, Artificial
  LRRC15 mRNA sequence
acacucaugcugcagccuugagccgucccucguccuccucucaggcucccucuuguccacggcgggcgggcgccgagcugcugaugugucccuagcauaaccuggccccauuccuuugcaucuagaugccuuuggacaaggcuaugccacugaagcauuaucuccuuuugcuggugggcugccaagccuggggugcaggguuggccuaccauggcugcccuagcgaguguaccugcuccagggccucccagguggagugcaccggggcacgcauuguggcggugcccaccccucugcccuggaacgccaugagccugcagauccucaacacgcacaucacugaacucaaugaguccccguuccucaauaucucagcccucaucgcccugaggauugagaagaaugagcugucgcgcaucacgccuggggccuuccgaaaccugggcucgcugcgcuaucucagccucgccaacaacaagcugcagguucugcccaucggccucuuccagggccuggacagccuugagucucuccuucuguccaguaaccagcuguugcagauccagccggcccacuucucccagugcagcaaccucaaggagcugcaguugcacggcaaccaccuggaauacaucccugacggagccuucgaccaccugguaggacucacgaagcucaaucugggcaagaauagccucacccacaucucacccagggucuuccagcaccugggcaaucuccagguccuccggcuguaugagaacaggcucacggauauccccaugggcacuuuugaugggcuuguuaaccugcaggaacuggcucuacagcagaaccagauuggacugcucuccccuggucucuuccacaacaaccacaaccuccagagacucuaccuguccaacaaccacaucucccagcugccacccagcaucuucaugcagcugccccagcucaaccgucuuacucucuuugggaauucccugaaggagcucucucuggggaucuucgggcccaugcccaaccugcgggagcuuuggcucuaugacaaccacaucucuucucuacccgacaaugucuucagcaaccuccgccaguugcagguccugauucuuagccgcaaucagaucagcuucaucuccccgggugccuucaacgggcuaacggagcuucgggagcugucccuccacaccaacgcacugcaggaccuggacgggaaugucuuccgcauguuggccaaccugcagaacaucucccugcagaacaaucgccucagacagcucccagggaauaucuucgccaacgucaauggccucauggccauccagcugcagaacaaccagcuggagaacuugccccucggcaucuucgaucaccuggggaaacugugugagcugcggcuguaugacaaucccuggaggugugacucagacauccuuccgcuccgcaacuggcuccugcucaaccagccuagguuagggacggacacuguaccuguguguuucagcccagccaauguccgaggccagucccucauuaucaucaaugucaacguugcuguuccaagcguccaugucccugaggugccuaguuacccagaaacaccaugguacccagacacacccaguuacccugacaccacauccgucucuucuaccacugagcuaaccagcccuguggaagacuacacugaucugacuaccauucaggucacugaugaccgcagcguuuggggcaugacccaggcccagagcgggcuggccauugccgccauuguaauuggcauugucgcccuggccugcucccuggcugccugcgucggcuguugcugcugcaagaagaggagccaagcuguccugaugcagaugaaggcacccaaugaguguuaaagaggcaggcuggagcagggcuggggaaugaugggacuggaggaccugggaauuucaucuuucugccuccaccccuggguccauggagcuuucccgugauugcucuuucuggcccuagauaaaggugugccuaccucuuccugacuugccugauucucccguagagaagcaggucgugccggaccuuccuacaaucaggaagauagauccaacuggccauggcaaaagcccuggggauuuccgauucauaccccugggcuuccuucgagagggcucuuccuccaaauccuccccaccuguccuccaagaacagccuucccugcgcccaggcccccuccgggccucuguagacucaguuaguccacagccugcucacuucgugggaauaguucuccgcugagauagccccucucgccuaaguauuauguaaguugauuucccuucuuuuguuucucuuguuugugcuauggcuugacccagcauguccccucaaaugaaaguucuccccuugauuuucugcuccugaaggcagggugaguucucuccucaaagaagacuucaaaccauuuaacugguuucuuaagagccgucaaucagccugguuuuggggaugcuaugaaagagagaaggaaaaucaugccgcucaguuccuggagacagaagagccgucaucagugucucacuugugauuuuuaucuggaaaaggaagaaacaccccagcacagcaagcucagccuuuuagagaaggauauuuccaaacugcaaacuuugcuuugaaaaguuuagcccuuuaaggaaugaaaucauguagaauuuuggacuucuaaaaacauuaaaaucagcuuauuaauacgggauagagaaagaaaucuggugccugggggucccuguguucaccccuagaguuuguuuuaaaauuuuuaauugaagcaugugaaguguaccugcagaaaagugggaacaugauaguguauggcuugguggauuuucacaaacugaacauaccuguguaaucagcaucuagacccagacccagagcgucacaaauaucccccauccugggcuuuucccagaggagaugggggcuucugaagauggacuuaccugggaccugccccccaugagccaggacgguccccccacagucagccugugcaaaggccccguggccagggguggaggagaauauguggguguggacaggaugggagacuguggccugaacaggagauuuuauuauaucuggagacccugagagacccugagaccuggggcacccuggcuggccaggucagaagcauccugacugcagagguccgugcagccacacccucuucccugccagcaagcugucugcggcucaucggaggccccuccgccuggagccuucuauggacgugauaugccuguaucuguuuuuaauuuucauucuucacuuaggggaagugaaaucgcucagagaugagauccuuuaauugaaaacgaaguguaacggaaucuagugucuuucuaaugugguaaaauucuccaucaacaucacagucagcuggcagcugaacuucagaaucucacuuacagcaggcgacacggggguacaccgaugggucacacugggucugggggcucccuggagcuccuccugcguguggucugguuaggaguugaguuguuugcuccaggguuauucuccuccucgagucacagucacacgaauaccugccuucucuggcuuuccugcuauacacauauucacauggcgcucaagaaguuaggcucauggcaacgugugucuuucucuggacaacuggcccaguuuacagugaaauggagaauuucaggucuccacgucugcccaggaaagaacuucagcugacuccacggggaucuggaaauccacgaccaaucccgaucggcucuuauuagcuccccgcuccacaagacaccugugcuuuggaaauccaccaccaaucccgaucggcucuuauuagcuccccgcuccacaagacaccugugaucuggaaaucuaccaccaaucccgaucggcucuuauuagcuccccgcuccacaagacaccugugacauccuccagggccacaggagcacgugcugaccaguuuucccuuccaguuccugcacaaaaaguguccagagggcuguuugcaaacacuagugcacuuuguagcuuuucacccucugucccagggaaucuaggagagaugaggcccgucagagucaagagaugucauccccccagggucuccaaggcauuuccacacuauugguggcaccuggaggacaugcaccaaggcuugccagagccaacaggaagugagcccagagcauggcacaugagcaucacccgcugaugguggccugcugugccuggugccaacaggggcaucccggcccauaccccuccagacaggaagcauggguuugcccacagaccugucgggugcuccugugaguggccuccagaugucuuugugcauaggcacaagugggccagggcuggagggaggugggaaaccucaucauccggugggcccugccaaucuuaacccagaacccuuagguauuccuggcaguagccaugacauuggagcaccuuccucuccagccagaggcugaccugagggccacuguccucagaugacaccacccaggagcacccuaggugaggggugagggcccccuuaugugaaccucuugccucuuccuuucucccaucagagugguuggauggagccauuggccuccuuuucuucagcgggcccuucaaccucucugcaccauguugucuggcugaggagcuacuagaaaagcugaguggagucuccuuuccaacaggaugaugcauuugcucaauucucagggcuggaaugagccggcuggucccccagaaagcuggagugggguacagaguucaguuuuccucucuguuuacagcuccuugacagucccacgcccaucuggagugggagcugggagucaguguuggagaagaaacaacaaaagccaauuagaaccacuauuuuuaaaaagugcuuacugugcacagauacucuucaagcacuggacguggauucucucucuagcccucagcaccccugcgguaggagugccgccucuacccacuugugaugggguacagaggcacuugcucuucugcaugguguucaauaggcugggaguuuuauuuaucucuucaaacuuuguacaagagcucauggcuugucuugggcuuucgucauuaaaccaaaggaaauggaagccauuccccuguugcucuccuuagucuuggucaucagaaccucacuugguaccauauagaucaaaagcuuuguaaccacaggaaaaaauaaacucuuccaucccuuaaagaauagaauaguuugucccucucaugggaauugggcuguauguauauuguucuuccuccuuagaauuuagagauacaagaguucuacuuagaacuuuucauggacacaauuuccacaaccuuucagaugcugauguagagcuauugggaaagaacuuccaaacucaggaaguuugcagagagcagacagcuagagauaacucgggacccagaguuggucgacagauguuagauguauccuagcuuuuagcuauaaaccacucaaagauucagcccccagaucccacagucagaacugaaucugcguuguugggaagccagcaguggccuugggaaggaagccauggcugugguucagagagggugggcuggcaagccacuuccggggaaaacuccuuccgccccagguuucuucuucucuuaaggagagauuauucucaccaacccgcugccuucaugcugccuucaaagcuagaucauguuugccuugcuuagagaauuacugcaaaucagccccagugcuuggcgaugcauuuacagauuucuaggcccucaggguuuuguagagugugagcccuggugggcaggguuggggggucugucuucugcuggaugcugcuuguaauccauuugguguacagaaucaacaauaaauaauauacaugua

SEQ ID NO: 6 - DNA, 25 bp, Artificial
  Primer that amplifies a PCR product of 391bp from any cDNA template containing the Lib gene
gacgggaatgtcttccgcatgttgg

SEQ ID NO: 7 - DNA, 25 bp, Artificial
  Primer that amplifies a PCR product of 391bp from any cDNA template containing the Lib gene
tttctgggtaactaggcacctcagg

SEQ ID NO: 8 - DNA, 20 bp, Artificial
  Primer used for amplification of the GAPDH gene
accacagtccatgccatcac

SEQ ID NO: 9 - DNA, 20 bp, Artificial
  Primer used for amplification of the GAPDH gene
tccaccaccctgttgctgta

SEQ ID NO: 10 - DNA, 21 bp, Artificial
  PCR primer used for detecting human Slit-Like 2 gene expression
acagacagtcttctgcactgc

SEQ ID NO: 11 - DNA, 21 bp, Artificial
  PCR primer used for detecting human Slit-Like 2 gene expression
tcgttgtcctgcagcttgagc

SEQ ID NO: 12 - DNA, 40 bp, Artificial
  PCR forward primer from the breast cancer cell line ME-180 with desired restriction site
actactgctagcttaacactcattgggtgccttcatctgc

SEQ ID NO: 13 - DNA, 45 bp, Artificial
  PCR reverse primer from the breast cancer cell line ME-180 with desired restriction site
actactgtcgacctcaccatgccactgaagcattatctcctttgc

SEQ ID NO: 14 - protein, 7 aa, Artificial
  TEV protease site
ENLYFQG

SEQ ID NO: 15 - DNA, 40 bp, Artificial
  P1 forward primer used for amplifying LRRC15 amino terminal extra cellular domain fragmen
gcggcgagatctctcaccatgccactgaagcattatctcc

SEQ ID NO: 16 - DNA, 21 bp, Artificial
  P2 reverse primer used for amplifying LRRC15 amino terminal extra cellular domain fragmen
gagagactcgaggcttgtcca

SEQ ID NO: 17 - DNA, 19 bp, Artificial
  P3 forward primer used for amplifying LRRC15 carboxyl terminal extracellular domain fragmen
caaccagcctaggttaggg

SEQ ID NO: 18 - DNA, 56 bp, Artificial
  P4 reverse primer used for amplifying LRRC15 carboxyl terminal extracellular domain fragmen
gcggcggctagcgccctggaagtacaggttctcgctctgggcctgggtcatgcccc

SEQ ID NO: 19 - DNA, 20 bp, Artificial
  Primer used for synthesis of cDNA
atgccactgaagcattatct

SEQ ID NO: 20 - DNA, 20 bp, Artificial
  Primer used in the synthesis of cDNA
gctccgtcagggatgtattc

SEQ ID NO: 21 - protein, 32 aa, Artificial
  Peptides derived from the extracellular domain of LRRC15
CLESLLLSSNQLLQIQPAHFSQSSNLKELQLH

SEQ ID NO: 22 - protein, 31 aa, Artificial
  Peptides derived from the extracellular domain of LRRC15
EVPSYPETPWYPDTPSYPDTTSVSSTTELTC

(19) United States
(12) Patent Application Publication
(10) Pub. No.: US 2005/0239700 A1
(43) Pub. Date: Oct. 27, 2005

(54) Treatment of cancer using antibodies to LRRC15

(75) Inventors: William S. Kloetzer, Carlsbad, CA (US); Karen McLachlan, Encinitas, CA (US); Michael I. La Barre, San Diego, CA (US); Jonathon Fitchett, San Marcos, CA (US); Robert Peach, San Diego, CA (US); Bill Shestowsky, Encinitas, CA (US); Scott Glaser, San Diego, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Biogen Idec Inc.

(21) Appl. No.: 10/963,987

(22) Filed: Oct. 14, 2004

Related U.S. Application Data
(60) Provisional application No. 60/510,552, filed on Oct. 14, 2003.

(51) Int. Cl. A61K 38/17; A61K 48/00; A61K 39/395

(52) U.S. Cl. 514/12; 514/44; 424/155.1

(57) ABSTRACT

The present invention is directed to novel methods of treating or diagnosing a hyperproliferative disease or disorder in an patient, where the methods include administrating to the patient a binding molecule which binds to a cell surface-expressed glycoprotein expressed predominantly in tumor or tumor-associated cells. In particular, the therapeutic and diagnostic methods of the present invention include the use of a binding molecule, for example an antibody or immunospecific fragment thereof, which specifically binds to the human LRRC15 protein. The present invention further provides a method of isolating and identifying cell surface expressed glycoproteins expressed in tumor or tumor associated tissues, where the method includes isolating desired glycoproteins via their affinity for specific lectins.

### CROSS REFERENCE TO RELATED APPLICATIONS

Claims the benefit of U.S. Provisional Application No. 60/510,552, filed Oct. 14, 2003, which is incorporated herein by reference in its entirety.

### BACKGROUND OF THE INVENTION

1. Field of the Invention

The present invention is directed to novel methods of treating hyperproliferative disorders utilizing binding molecules which bind to polypeptides expressed predominantly in tumor or tumor-associated cells.

2. Background Art

Leucine-rich repeats (LRRs) are 20-29-residue sequence motifs present in a number of proteins with diverse functions such as hormone-receptor interactions, enzyme inhibition, cell adhesion and cellular trafficking. The primary function of these motifs appears to be to provide a versatile structural framework for the formation of protein-protein interactions. A number of recent studies revealed the involvement of LRR proteins in early mammalian development, neural development, cell polarization, regulation of gene expression and apoptosis signaling. In addition, LRRs may be critical to the morphology and dynamics of cytoskeleton. See, e.g., Kobe B., and Kajava A. V., Curr. Opin. Struct. Biol. 11: 725-732 (2001), which is incorporated herein by reference in its entirety.

LRRC15 is a leucine-rich transmembrane protein of 581 amino acids. See, e.g., Genbank Accession Number Q8TF66. The full cDNA sequence of 5938 bases is available as Genbank Accession Number BK001325. See also, Reynolds, P. A., et al., Genes Dev. 17:2094-2107 (2003), and Satoh, K., et al., Biochem. Biophys. Res. Commun. 290:756-762 (2002).

Reynolds et al., noted that LRRC15 is normally expressed on the leading edge of invasive cells in the placental cytotrophoblast layer, but showed that its expression was dramatically upregulated by a transcription factor expressed in desmoplastic small round cell tumor cells. Reynolds et al. further demonstrated that siRNA-mediated suppression of LRRC15 expression in breast cancer cells lead to abrogation of invasiveness in an in vitro system, hypothesizing that LRRC15 may be generally involved in tumor invasiveness. Expression of the rat homolog of LRRC15, designated LIB, was induced in rat astrocytes in response to beta-amyloid protein. See Satoh et al.

### BRIEF SUMMARY OF THE INVENTION

This invention involves using lectins that bind selectively to carbohydrate moieties of glycoproteins to purify vascular proteins that are present in normal tissues and in tissues associated with a disease or disorder. Such disease- or disorder-associated vascular tissues are present, for example, in tumor-bearing and inflammatory tissues. The invention also involves using protein separation and sequencing methods to determine amino acid sequences of peptides derived from lectin-binding proteins of such tissue samples, and comparing the peptide sequences to databases containing known protein amino acid sequences to identify the purified proteins, and to further identify proteins that are specifically present in vascular tissues associated with a disease or disorder. When proteins of non-human tissues are identified, the invention also involves comparing the amino acid sequences of such proteins to the databases to identify their human homologs. The invention further involves preparing therapeutic agents such as monoclonal antibodies and fusion proteins bearing extracellular binding domains that bind with high affinity and specificity to proteins that are specifically present in disease- or disorder-associated tissues, e.g., proteins that are useful targets for killing or interfering with the function of cells of the tissue that express the targeted proteins.

Human target proteins which were identified by this method include CDO, TMEFF2, KIAA1484, LRRN3, LRRC15, synaptogyrin 3, and Slit-like 2 protein.

In one embodiment, the present invention provides a method for treating a hyperproliferative disorder in an animal, comprising administering to an animal in need of treatment a composition comprising a binding molecule which specifically binds to an LRRC15 gene product.

Yet another embodiment provides a method of detecting abnormal hyperproliferative cell growth in a patient, comprising: obtaining a biological sample from the patient; contacting the sample with a binding molecule which specifically binds to an LRRC15 gene product, and assaying the expression level of LRRC15 transcript or polypeptide in the sample.

A method of diagnosing a hyperproliferative disease or disorder in a patient, comprising administering to the patient a sufficient amount of a detectably labeled binding molecule which specifically binds to an LRRC15 gene product, waiting for a time interval following the administration to allow the binding molecule to contact the LRRC15 gene product and detecting the amount of binding molecule which is bound to thegene productin the patient.

In various embodiments, binding molecules for use in the above methods include antibodies and antigen-specific fragments thereof, fusion proteins, T-cell receptors, antisense nucleic acids, siRNAs, ribozymes, and small molecules.

In the above methods, binding molecules bind to gene products including, but not limited to an LRRC15 polypeptide comprising, consisting essentially of or consisting of amino acids 1 to 581 of SEQ ID NO:2 or a fragment thereof, an LRRC15 variant polypeptide at least 70% identical to an LRRC15 polypeptide fragment consisting essentially of amino acids 22 to 581 of SEQ ID NO:2, an LRRC15 variant polypeptide at least 70% identical to an LRRC15 polypeptide fragment consisting essentially of amino acids 22 to 537 of SEQ ID NO:2, an LRRC15 variant polypeptide at least 70% identical to an LRRC15 polypeptide fragment consisting essentially of amino acids 22 to 538 of SEQ ID NO:2, and an LRRC15 messenger RNA consisting essentially of nucleotides 1 to 5938 of SEQ ID NO:5 or a fragment thereof;

### BRIEF DESCRIPTION OF THE DRAWINGS/FIGURES

FIG. 1A: DNA sequence of the coding region of human LRRC15; FIG. 1B: amino acid sequence of human LRRC15.

FIG. 2A: LRRC15-Tev-Fc fusion protein DNA sequence; FIG. 2B: LRRC15-Tev-Fc fusion protein amino acid sequence

FIG. 3A. Biotin-GSIB4 staining of a sectioned HT29 tumor xenograft. The box encloses a sectioned blood vessel. 4× magnification.

FIG. 3B. Gal-blocked biotin-GSIB4 staining of a sectioned HT29 tumor xenograft. Human cells are counterstained blue with monoclonal anti-human EPCAM antibody. 4× magnification.

FIG. 3C. Biotin-GS1134 staining of a sectioned HT29 tumor xenograft. Human cells are counterstained blue with anti-human EPCAM antibody. 4× magnification.

FIG. 3D. Biotin-GSIB4 staining of a sectioned HT29 tumor xenograft. 20× magnification.

FIG. 3E. Biotin-GSIB4 staining of a sectioned HT29 tumor xenograft, with blue counterstaining of the human cells with anti-human EPCAM antibody 20× magnification.

FIG. 3F. Gal-blocked biotin-GSE34 staining of a sectioned HT29 tumor xenograft. Human cells are counterstained blue with anti-human EPCAM antibody 20× magnification.

FIG. 3G. Biotin-GSIB4 staining of sectioned normal lung vasculature of SCID-bg mouse. 40× magnification.

FIG. 3H. Biotin-GSIB4 staining of sectioned normal lung vasculature of SCID-bg mouse 10× magnification.

FIG. 3I. Gal-blocked biotin-GSIB4 staining of sectioned normal lung vasculature of SCID-bg mouse 10× magnification.

FIG. 4: Detection of murine endothelial cell-specific Flk-1 (VEGFR2) in proteins of normal SCID mouse lung homogenate that were adsorbed to and eluted from agarose GSIB4, after separation in a 4-20% SDS Tris-glycine reducing gel. Lanes 1 and 2 are colloidal blue-stained gel. Lanes 3 and 4 are stained with anfi-Flk-1 by Western blot. Lanes 5 and 6 are stained with anti-Flk-1 by Western blot with peptide blocking.

Leftmost lane: molecular weight markers.

Lanes 1, 3, and 5: agarose GSIB4 standard.

Lanes 2, 4, and 6: 4 mg normal SCID mouse lung homogenate.

FIG. 5: Detection of murine endothelial cell-specific Flk-1 (VEGFR2) in proteins of normal SCID mouse lung homogenate that were first adsorbed to agarose GSIB4, then to agarose-Concanavalin A, after separation in a 4-20% SDS Tris-glycine reducing gel. Lanes 1-3 are colloidal blue-stained gel. Lanes 4-6 are stained with anti-Flk-1 by Western blot.

Lanes 1 and 4: 4 mg normal SCID mouse lung homogenate.

Lanes 2 and 5: 4 mg normal SCID mouse lung homogenate (second preparation).

Lanes 3 and 6: agarose GSIB4 standard.

Lane between lanes 2 and 3: molecular weight markers.

FIG. 6: Detection of proteins of HT29 xenograft-associated tissue and normal SCID mouse lung that were first adsorbed to agarose GSE34, eluted with 0.2M galactose, and then adsorbed to and eluted from agarose-Concanavalin A, followed by separation in a 4-20% SDS Tris-glycine reducing gel and staining with colloidal blue. The gels were subsequently sliced and the proteins eluted for MS-MS analysis.

Lane 1: Gal-eluted agarose-GSIB4 pellet of protein from normal SCID-bg mouse lung.

Lane 2: Gal-eluted agarose-GSIB4 pellet of protein from subcutaneous HT29 xenograft-associated tissue.

Lane 3: agarose-Con A.

Lane 4: agarose-Con A pellet of protein from normal SCID-bg mouse lung.

Lane 5: agarose-Con-A pellet of protein from subcutaneous HT29 xenograft-associated tissue.

Lane between lanes 3 and 4: molecular weight markers.

FIG. 7: Murine GSE34-binding proteins witb transmembrane (TM) domains purified from HT29 xenograft-associated tissue.

FIG. 8A: Graphic representation of predicted structural features of the 391 amino acid murine LIB fragment.

FIG. 8B: Graphic representation of predicted structural features of the 579 amino acid murine LIB polypeptide.

FIG. 9A: Schematic representation of predicted structural features of the complete murine LIB polypeptide.

FIG. 9B: Schematic representation of predicted structural features of the 581 amino acid human LRRC15 polypeptide.

FIG. 10 Detection of expression of human LRRC15 in human multiple tissue panels I and II by RT-PCR.

FIG. 11 Detection of expression of human LRRC15 in human cardiovascular tissue panel by RT-PCR.

FIG. 12 Detection of expression of human LRRC15 in human tumor panels by RT-PCR. A: Multiple tumor panel; B: Colon and prostate panel

FIG. 13 Detection of expression of human LRRC15 in matched tumor tissues and normal tissues derived from human lung by RT-PCR.

FIG. 14 Detection of expression of human LRRC15 in matched tumor tissues and normal tissues derived from human breast (panel A) and ovary (panel B) by RT-PCR.

FIG. 15 Detection of expression of human LRRC15 in normal human cell lines by RT-PCR.

FIG. 16 Detection of expression of a human LRRC15 in human tumor cell lines by RT-PCR.

FIG. 17: Detection of expression of a human Slit-like 2 gene in human cells and tissues by RT-PCR.

FIG. 18A: Biotin-UEA1 staining of sectioned human normal kidney tissue N33, showing selective staining of glomeruli and other renal vasculature by biotin-UEA1.

FIG. 18B: Biotin-UEA1 staining of sectioned human normal kidney tissue N35, showing selective staining of glomeruli and other renal vasculature, by biotin-UEA1.

FIG. 18C: Biotin-UEA1 staining of sectioned human tumor-bearing kidney tissue T16, showing selective staining of human kidney tumor vasculature by biotin-UEA1.

FIG. 18D: Biotin-UEA1 staining of sectioned human tumor-bearing kidney tissue T27, showing selective staining of human kidney tumor vasculature by biotin-UEA1.

FIG. 18E: Biotin-UEA1 staining of sectioned human tumor-bearing kidney tissue T36, showing selective staining of human kidney tumor vasculature by biotin-UEA1.

FIG. 18F: Biotin-UEA1 staining of sectioned human tumor-bearing kidney tissue T37, showing selective staining of human kidney tumor vasculature by biotin-UEA1.

FIG. 19A: Biotin-UEA1 staining of sectioned human normal kidney tissue N33, showing selective staining of glomeruli and other renal vasculature by biotin-UEA1. 10× magnification.

FIG. 19B: Biotin-UEA1 staining of sectioned human normal kidney tissue N33, showing selective staining of glomeruli and other renal vasculature by biotin-UEA1 20× magnification.

FIG. 19C: Biotin-UEA1 staining of sectioned human normal kidney tissue N33, showing selective staining of glomeruli and other renal vasculature by biotin-UEA1. 40× magnification.

FIG. 19D: Biotin-UEA1 staining of sectioned human normal kidney tissue N33, showing selective staining of glomeruli and other renal vasculature by biotin-UEA1. 60× magnification.

FIG. 20A: Biotin-UEA1 staining of sectioned human tumor-bearing kidney tissue T27, showing selective staining of human kidney tumor vasculature by biotin-UEA1. 10× magnification.

FIG. 20B: Biotin-UEA1 staining of sectioned human tumor-bearing kidney tissue T27, showing selective staining of human kidney tumor vasculature by biotin-UEA1. 20× magnification.

FIG. 20C: Biotin-UEA1 staining of sectioned human tumor-bearing kidney tissue T27, showing selective staining of human kidney tumor vasculature by biotin-UEA1. 40× magnification.

FIG. 20D: Biotin-UEA1 staining of sectioned human tumor-bearing kidney tissue T27, showing selective staining of human kidney tumor vasculature by biotin-UEA1. 60× magnification.

FIG. 21: RT-PCR analysis of CHO cell lines expressing full length human LRRC15.

FIG. 22: Western blot of human LRRC15-Tev-Fc fusion protein expression.

FIG. 23: 4-20% Tris Glycine SDS PAGE gel showing expression of a human LRRC15-Tev-Fc fusion protein. Lane 1: size standards; lane 2: protein sample non-reduced; lane 3: blank; and lane 4: protein sample reduced.

FIG. 24: Serum titers from mice immunized with human LRRC15-derived peptides 1 and 2. KEY: NMS=normal mouse serum, ##1-3, 10 and 20 are serum samples from individual immunized mice.

FIG. 25: Mass Spectrometry Analysis of HT 29 Xenograph for Cut 6 (Lib). A. Total Ion Chromatograph for HT 29 Xenograph B. MS spectrum for Cut 6. C. MSMS spectrum for Lib. Ions used to identify the Lib peptide are noted.

FIG. 26: Sequence of the LRRC15 messenger RNA, derived from GenBank BK001325.

### DETAILED DESCRIPTION OF THE INVENTION

I. Definitions

Binding molecules. The methods of treating hyperproliferative disorders as described herein utilize “binding molecules.” A binding molecule comprises, consists essentially of, or consists of at least one binding domain which, either alone or in combination with one or more additional binding domains, specifically binds to a target gene product (such as a messenger RNA, a protein, an antigen or other binding partner), e.g., a transcript encoding LRRC15 or a fragment thereof or an LRRC15 polypeptide or fragment or variant thereof. For example, in various embodiments, a binding molecule comprises one or more antisense nucleic acids, one or more siRNA molecules, one or more ribozymes, one or more immunoglobulin antigen binding domains, one or more binding domains of a receptor molecule which, either alone or together, specifically bind a ligand, or one or more binding domains of a ligand molecule which, either alone or together, specifically bind a receptor. Nucleic acid binding molecules are described in more detail below. In certain embodiments, a binding molecule comprises, consists essentially of, or consists of at least two binding domains, for example, two, three, four, five, six, or more binding domains. Each binding domain may bind to a target molecule separately, or two or more binding domains may be required to bind to a given target, for example, a combination of an immunoglobulin heavy chain and an immunoglobulin light chain.

Binding molecules, e.g., binding polypeptides, e.g., LRRC15-specific antibodies used in the diagnostic and treatment methods disclosed herein may comprise, consist essentially of, or consist of two or more subunits thus forming multimers, e.g., dimers, trimers or tetramers. For example, certain binding molecules comprise a polypeptide dimer, typically, a heterodimer comprising two non-identical monomeric subunits. Other binding molecules comprise tetramers, which can include two pairs of homodimers, e.g., two identical monomeric subunits, e.g., an antibody molecule consisting of two identical heavy chains and two identical light chains.

Certain binding molecules, e.g., binding polypeptides to be utilized in the diagnostic and treatment methods disclosed herein comprise at least one amino acid sequence derived from an immunoglobulin. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the starting sequence. Alternatively, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence, as described in more detail below.

Preferred binding polypeptides comprise, consist essentially of, or consist of an amino acid sequence derived from a human amino acid sequence. However, binding polypeptides may comprise one or more contiguous amino acids derived from another mammalian species. For example, a primate heavy chain portion, hinge portion, or binding site may be included in the subject binding polypeptides. Alternatively, one or more murine-derived amino acids may be present in a non-murine binding polypeptide, e.g., in an antigen binding site of a binding molecule. In therapeutic applications, preferred binding molecules to be used in the methods of the invention are not immunogenic in the animal to which the binding polypeptide is administered.

It will also be understood by one of ordinary skill in the art that the binding polypeptides for use in the diagnostic and treatment methods disclosed herein may be modified such that they vary in amino acid sequence from the naturally occurring binding polypeptide from which they were derived. For example, nucleotide or amino acid substitutions leading to conservative substitutions or changes at “non-essential” amino acid residues may be made.

In certain embodiments, a binding polypeptide for use in the methods of the invention comprises an amino acid sequence or one or more moieties not normally associated with that binding polypeptide. Exemplary modifications are described in more detail below. For example, a binding polypeptide of the invention may comprise a flexible linker sequence, or may be modified to add a functional moiety (e.g., PEG, a drug, a toxin, or a label).

A binding polypeptide for use in the methods of the invention may comprise, consist essentially of, or consist of a fusion protein. Fusion proteins are chimeric molecules which comprise a binding domain with at least one target binding site, and at least one heterologous portion.

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the rest of the entity to which it is being compared. For instance, a heterologous polynucleotide or antigen may be derived from a different species origin, different cell type, or the same type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to any native ligand or any region or derivative thereof retaining at least a qualitative receptor binding ability, and preferably the biological activity of a corresponding native ligand.

Antibody or Immunoglobulin. In one embodiment, the binding molecules for use in the diagnostic and treatment methods disclosed herein are “antibody” or “immunoglobulin” molecules, or immunospecific fragments thereof, e.g., naturally occurring antibody or immunoglobulin molecules or engineered antibody molecules or fragments that bind antigen in a manner similar to antibody molecules. The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises five broad classes of polypeptides that can be distinguished biochemically. All five classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H}3 and C_{L} domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In camelid species, however, the heavy chain variable region, referred to as V_{H}H, forms the entire CDR. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR3 in V_{H}H, and (c) the frequent occurrence of a disulfide bond between CDR1 and CDR3 in V_{H}H.

In one embodiment, an antigen binding molecule of the invention comprises at least one heavy or light chain CDR of an antibody molecule. In another embodiment, an antigen binding molecule of the invention comprises at least two CDRs from one or more antibody molecules. In another embodiment, an antigen binding molecule of the invention comprises at least three CDRs from one or more antibody molecules. In another embodiment, an antigen binding molecule of the invention comprises at least four CDRs from one or more antibody molecules. In another embodiment, an antigen binding molecule of the invention comprises at least five CDRs from one or more antibody molecules. In another embodiment, an antigen binding molecule of the invention comprises at least six CDRs from one or more antibody molecules. Exemplary antibody molecules comprising at least one CDR that can be included in the subject antigen binding molecules are known in the art and exemplary molecules are described herein.

Antibodies or immunospecific fragments thereof for use in the methods of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to binding molecules disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain binding molecules, e.g., binding polypeptides, e.g., LRRC15-specific antibodies or immunospecific fragments thereof for use in the diagnostic and treatment methods disclosed herein, the heavy chain portions of one polypeptide chain of a multimer are identical to those on a second polypeptide chain of the multimer. Alternatively, heavy chain portion-containing monomers for use in the methods of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a C_{H}1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a V_{L} or C_{L} domain.

An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into binding molecules for use in the diagnostic and treatment methods disclosed herein and screened for their ability to bind to the desired antigen, e.g., LRRC15.

Antibodies or fragment thereof for use in the diagnostic and therapeutic methods disclosed herein may be described or specified in terms of the epitope(s) or portion(s) of a target polypeptide that they recognize or specifically bind. The portion of an antigen which specifically interacts with the antigen binding domain of an antibody is an “epitope,” or an “antigenic determinant.” An antigen may comprise a single epitope, but typically, an antigen comprises at least two epitopes, and can include any number of epitopes, depending on the size, conformation, and type of antigen. Antigens are typically peptides or polypeptides, but can be any molecule or compound or a combination of molecules or compounds. For example, an organic compound, e.g., dinitrophenol or DNP, a nucleic acid, a carbohydrate, or a mixture of any of these compounds either with or without a peptide or polypeptide can be a suitable antigen. Thus, for example, an “epitope” on a polypeptide may include a carbohydrate side chain.

The minimum size of a peptide or polypeptide epitope is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide antigens preferably contain a sequence of at least 4, at least 5, at least 6, at least 7, more preferably at least 8, at least 9, at least 10, at least 15, at least 20, at least 25, and, most preferably, between about 15 to about 30 amino acids. Preferred peptides or polypeptides comprising, or alternatively consisting of, antigenic epitopes are at least 10, 15, 20, 25, 30, 35, 40, 45, 50, 55, 60, 65, 70, 75, 80, 85, 90, 95, or 100 amino acid residues in length.

By “specifically binds,” it is generally meant that an antibody binds to an epitope via its CDR, and that the binding entails some complementarity between the CDR and the epitope. According to this definition, an antibody is said to “specifically bind” to an epitope when it binds to that epitope, via its CDR more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody for use in the diagnostic and treatment methods disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An antibody or fragment thereof for use in the diagnostic and treatment methods disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Antibodies or immunospecific fragments thereof for use in the diagnostic and therapeutic methods disclosed herein may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Antibodies or immunospecific fragments thereof for use in the diagnostic and treatment methods disclosed herein may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹M, 5×10⁻¹⁰ M, 10⁻¹⁰ M, 5×10⁻¹¹ M, 10⁻¹¹ M, 5×10⁻¹²M, 10⁻¹² M, 5×10⁻¹³ M, 10¹³ M, 5×10⁻¹⁴ M, 10⁻¹⁴ M, 5×10⁻¹⁵ M, or 10⁻¹⁵ M.

Antibodies or immunospecific fragments thereof for use in the diagnostic and treatment methods disclosed herein may act as agonists or antagonists of target polypeptides described herein. For example, an antibody for use in the methods of the present invention may function as an antagonist, blocking or inhibiting LRRC15 activity.

As used herein, the term “binding site” or “binding domain” refers to a region of a binding molecule, e.g., a binding polypeptide, e.g., an antibody or fragment thereof, which is responsible for specifically binding to a target molecule of interest (e.g., an antigen, ligand, receptor, substrate or inhibitor) Exemplary binding domains include antibody variable domains, a receptor binding domain of a ligand, or a ligand binding domain of a receptor or an enzymatic domain. A binding domain on an antibody is referred to herein as an “antigen binding domain.”

A binding molecule, binding polypeptide, or antibody for use in the diagnostic and treatment methods disclosed herein may be “multispecific,” e.g., bispecific, trispecific or of greater multispecificity, meaning that it recognizes and binds to two or more different epitopes present on one or more different antigens (e.g., proteins) at the same time. Thus, whether a binding molecule is “monospecfic” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in a binding molecule, binding polypeptide or antibody. Each binding domain specifically binds one epitope. When a binding molecule, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incoporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “V_{H} domain” includes the amino terminal variable domain of an immunoglobulin heavy chain and the term “C_{H}1 domain” includes the first (most amino terminal) constant region domain of an immunoglobulin heavy chain. The C_{H}1 domain is adjacent to the V_{H} domain and is amino terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., antigen binding molecules such as antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

In one embodiment, a subject can be treated with a nucleic acid molecule encoding a binding molecule, e.g., a binding polypeptide, e.g., a LRRC15-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

In some embodiments, the present invention employs compositions comprising oligomeric antisense compounds, particularly oligonucleotides (e.g., those identified in the drug screening methods described above), for use in modulating the function of nucleic acid molecules encoding stem cell cancer markers of the present invention, ultimately modulating the amount of cancer marker expressed. This is accomplished by providing antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state. In the present invention, the target is a nucleic acid molecule encoding a tumor-associated cell-surface protein. The targeting process also includes determination of a site or sites within this gene for the antisense interaction to occur such that the desired effect, e.g., detection or modulation of expression of the protein, will result.

For example, regions for targeting with antisense include the regions encompassing the translation initiation codon, translation termination codon, or the open reading frame (ORF) of the gene. Since the translation initiation codon is typically 5′ AUG (in transcribed mRNA molecules; 5′ ATG in the corresponding DNA molecule), the translation initiation codon is also referred to as the “AUG codon,” the “start codon” or the “AUG start codon”. The translation termination codon (or “stop codon”) of a gene may have one of three sequences (i.e., 5′ UAA, 5′ UAG and 5′ UGA; the corresponding DNA sequences are 5′ TAA, 5′ TAG and 5′ TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. The open reading frame (ORF) or “coding region,” which refers to the region between the translation initiation codon and the translation termination codon, is also a region that may be targeted effectively.

Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7 methylated guanosine residue joined to the 5′ most residue of the mRNA via a 5′ 5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. Messenger RNA splice sites (i.e., intron exon junctions) may also be preferred target regions. It has also been found that introns can also be effective, and therefore preferred, target regions for antisense compounds targeted, for example, to DNA or pre mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publication No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non specific binding of the antisense compound to non target sequences under conditions in which specific binding is desired (i.e., under physiological conditions in the case of in vivo assays or therapeutic treatment, and in the case of in vitro assays, under conditions in which the assays are performed).

The term “standard hybridization conditions” refers to salt and temperature conditions substantially equivalent to 0.5×SSC to about 5×SSC and 65 degrees C. for both hybridization and wash. The term “standard hybridization conditions” as used herein is therefore an operational definition and encompasses a range of hybridization. Higher stringency conditions may, for example, include hybridizing with plaque screen buffer (0.2% polyvinylpyrrolidone, 0.2% Ficoll 400; 0.2% bovine serum albumin, 50 mM Tris-HCl (pH 7.5); 1M NaCl; 0.1% sodium pyrophosphate; 1% SDS); 10% dextran sulphate, and 100 μg/ml denatured, sonicated salmon sperm DNA at 65 degrees C. for 12-20 hours, and washing with 75 mM NaCl/7.5 mM sodium citrate (0.5×SSC)/1% SDS at 65 degrees C. Lower stringency conditions may, for example, include hybridizing with plaque screen buffer, 10% dextran sulphate and 110 μg/ml denatured, sonicated salmon sperm DNA at 55 degrees C. for 12-20 hours, and washing with 300 mM NaCl/30 mM sodium citrate (2.0×SSC)/1% SDS at 55 degrees C.

Antisense compounds are also commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

Ribozymes are RNA molecules possessing the ability to specifically cleave other single-stranded RNA in a manner analogous to DNA restriction endonucleases. Through the modification of nucleotide sequences which encode those RNA's, it is possible to engineer molecules that recognize and cleave specific nucleotide sequences in an RNA molecule (Cech, T. R., JAMA 260:3030-3034 (1988)). A major advantage of that approach is only mRNA's with particular sequences are inactivated.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant all neoplastic cell growth and proliferation, whether malignant or benign, incuding all transformed cells and tissues and all cancerous cells and tissues. Hyperproliferative diseases or disorders include, but are not limited to, precancerous lesions, abnormal cell growths, benign tumors, malignant tumors, and “cancer.”

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract.

Other hyperproliferative disorders include, but are not limited to:
hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult. Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976).

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

II. Identification of Disease- or Disorder-Associated Target Molecules

In one embodiment the present invention provides methods that utilize one or more lectins that bind selectively to mammalian glycoproteins as affinity reagents to identify, isolate, and/or purify vascular proteins that are expressed in a tissue of an animal or human in response to a disease or disorder; e.g., the presence of a tumor. Identification, isolation, or purification using the lectin as an affinity reagent can be performed, for example, by affinity chromatography directly from tissue homogenates, or by infusing conjugated lectins into a subject animal and allowing them to bind to the target proteins, then removing and homogenizing the tissue of interest and purifying the lectin-protein complexes.

Amino acid sequences of peptides derived from vascular proteins purified from normal tissue and from tissue that is affected by a disease or disorder are determined, e.g., by mass spectrometry microsequencing (MS-MS), and are then compared to known protein amino acid sequences to identify vascular proteins that are specifically expressed in the tissue in response to the disease or disorder. The amino acid sequences of non-human lectin-binding proteins that are identified in disease- or disorder-associated tissue but not in normal tissue are further compared to databases of known protein amino acid sequences to identify one or more human homologs. The specific cell types and sites in tissues in which the disease- or disorder-specific proteins are expressed in non-human animals or in humans are determined using known methods, for example, in situ hybridization (ISH), immunohistochemistry (IHC), and reverse transcription-polymerase chain reaction (RT-PCR).

This aspect of the invention is demonstrated using the lectin, Griffonia simplicifolia IB4 (GSIB4), which binds preferentially to glycoproteins of cells of non-primate vasculature. Tissues of a murine subcutaneous HT29 human tumor xenograft were removed and homogenized; and murine glycoproteins extracted from the tumor-associated tissues were isolated or purified by GSIB4 affinity chromatography. Murine proteins with affinity for GSIB4, that are expressed in a normal, vascularized mouse tissue (e.g., lung) were similarly isolated or purified. Peptide sequences of the GSIB4-binding proteins that were isolated from the tumor-associated tissue and the normal murine tissue were determined by MS-MS, and were compared to a database of known protein amino acid sequences to identify the isolated proteins. The amino acid sequences of selected murine proteins that were expressed in the tumor-associated tissue but not in the normal tissue were then compared to a database of human protein amino acid sequences, and their human homologs were identified.

Using known methods such as RT-PCR, human tissue(s) and cultured cells (e.g. endothelial cells and vascular smooth muscle cells) are evaluated for gene expression in order to assess the tumor specificity and cell type specificity of the GSIB4-binding proteins identified by the invention. Human homologs of tumor-associated proteins identified by the invention that are expressed as cell-surface proteins in tumor-induced vasculature but not in vasculature of normal tissue are selected for further validation as therapeutic targets.

The invention provides methods for identifying, isolating, and/or purifying tumor-related target polypeptides or proteins, e.g., unique tumor proteins or tumor-associated vascular and other cell-surface proteins that are produced by cells in an animal in tissues that show an altered or abnormal pattern of growth or development due to a disease or disorder, for example, in tumor-bearing and inflammatory tissues.

In particular, vascular tissues are induced by tumors to grow and develop abnormally so as to sustain the viability and growth of the tumor. Tumors induce angiogenesis, in which new blood vessels grow from the endothelium of existing blood vessels in a developed animal to provide blood to the growing tumor. in the absence of the blood supply provided by angiogenesis, cells of tumors larger than 1-2 mm³ are deprived of their blood supply, the tumor cells undergo apoptosis, and the tumors become necrotic.

In one embodiment, the present invention provides methods for identifying, isolating and purifying proteins that are expressed by a tumor cell or are present in blood vessels or other tumor-associated tissues induced by a tumor, but are not detected in normal vasculature or other normal tissues. “Identifying” a protein or a group of proteins refers to distinguishing certain proteins from other proteins, e.g., distinguishing glycoproteins expressed only in tumor- or tumor-associated tissues from glycoproteins which are also expressed in normal tissues. “Isolating” a protein refers to substantially removing such a protein from its natural milieu of proteins and other substances. The term “isolating” is not meant to require any level of purification. “Purifying” a protein refers to steps taken to prepare a homogeneous sample of the protein, i.e., procedures to remove a given protein from all other proteins or other substances.

The tumor- and tumor-associated proteins identified according to this method include cell surface proteins that are markers of tumor-induced cell differentiation and tissue formation. As described in more detail below, another embodiment of the present invention provides methods for the preparation and use of therapeutic agents, e.g., binding molecules, binding polypeptides, antibodies or fragment thereof, that bind to, and in certain embodiments antagonize, the tumor-induced proteins identified by methods disclosed herein, e.g., by binding specifically to the identified proteins or to ligands or receptors of the identified proteins. As further described below, yet another embodiment of the present invention provides methods for treating, ameliorating, preventing or blocking the invasiveness of a malignancy, e.g., a tumor or metastasis thereof, in an animal suffering from such a malignancy or predisposed to contract such a malignancy, the method comprising administering to the animal an effective amount of a binding molecule, binding polypeptide, or antibody that binds to a tumor- or tumor-associated protein identified by the methods described herein.

The methods of the invention for identifying, isolating and purifying proteins that are expressed by a tumor cell or are present in blood vessels or other tumor-associated tissues induced by a tumor use a lectin that binds selectively to vascular and other proteins produced by cells of an animal as an affinity purification agent to purify lectin-binding vascular and other proteins of the animal. Lectins are proteins that bind carbohydrate portions of glycoproteins and glycolipids. The invention can be practiced with any lectin that binds selectively to vascular or other glycoproteins produced by cells of an animal. A lectin that “binds selectively” to glycoproteins produced by cells of an animal is one that binds with moderate to high affinity to some glycoproteins of the animal, and with low or negligible affinity to other proteins of the animal. Degrees of binding affinity are described herein in an operative sense, and refer to affinity with which one molecule binds to another under solution conditions that are commonly used in affinity purification of proteins. The invention can be practiced with a lectin that binds selectively to vascular or other glycoproteins produced by cells of a tissue that shows an altered or abnormal pattern of growth or development due to a disease or disorder, for example, in a tumor-bearing or inflammatory tissue of an animal or human.

An example of a lectin that binds selectively to animal vascular glycoproteins is the lectin Griffonia simplicifolia IB4 (GSIB4), which binds selectively to glycoproteins of cells of vasculature of non-primate mammals, and is commonly used to histochemically stain vasculature of non-primate tissues. An example of a lectin that binds selectively to vascular glycoproteins of human tissue is the lectin Ulex europaeus agglutinin I (UEA1). Other lectins that bind selectively to vascular glycoproteins can also be used in the invention.

The invention provides methods for identifying, isolating, and purifying lectin-binding vascular or other proteins that are produced in a tissue that shows an altered or abnormal pattern of growth or development in an animal due to a disease or a disorder. For example, lectin-binding proteins that are present in tumor cells tumor-associated vascular tissue or other tumor-associated tissues are identified, isolated, and/or purified by a method that comprises the steps of
(a) homogenizing tissue that shows an abnormal pattern of growth or development due to the presence of a tumor in the animal (the first tissue),
(b) contacting proteins produced by cells in the tissue with molecules of a lectin that binds selectively to vascular glycoproteins produced by cells of the animal;
(c) separating the lectin-protein complexes from proteins and other cellular molecules that are not bound to the lectin molecules;
(d) contacting the lectin-protein complexes with an agent that effects dissociation of the lectin-protein complexes; and
(e) purifying the lectin-binding proteins from the lectin molecules.

The tissue containing the proteins of interest is homogenized using known methods. Proteins that are effective drug targets in vivo are often cell surface proteins that have a hydrophobic transmembrane domain. Such proteins can be solubilized efficiently in a homogenate that contains a detergent or surfactant. For example, transmembrane proteins are solubilized in homogenization solution comprising 1% Triton-X-100® (alpha-[4-(1,1,3,3-tetramethylbutyl)phenyl]-omega-hydroxypoly (oxy-1,2-ethanediyl), a non-ionic surfactant. Other detergents or surfactants that promote solubilization of transmembrane proteins are well known by persons skilled in the art.

The invention may also be practiced by contacting the proteins of the tissue with the lectin molecules prior to homogenization of the tissue. For example, the tissue can be contacted with the lectin molecules in vitro prior to homogenization; or the lectin molecules can be infused into the tissue in vivo prior to homogenization of the tissue. When the proteins of the tissue are contacted with the lectin molecules prior to homogenization of the tissue, it is useful to use lectin molecules that are coupled to a moiety that facilitates isolation and/or purification of lectin-protein complexes; e.g., a moiety that binds with high affinity to a ligand that can serve as an affinity purification agent of the moiety-lectin pair. For example, the proteins of the t issue can be contacted with biotinylated lectin molecules, and isolation or purification of the biotinylated lectin-protein complexes can be achieved by contacting the biotin moieties of the complexes with an avidin affinity purification matrix.

Alternatively, the invention may be practiced by removing the tissue that shows an altered or abnormal pattern of growth or development from the animal, and homogenizing the tissue before the proteins in the homogenate are contacted with the lectin molecules. In this embodiment, the lectin molecules can be covalently attached to support molecules such as agarose (e.g., agarose beads), and the lectin-binding proteins in the homogenate can be purified by lectin affinity chromatography. Proteins that bind non-specifically to the support molecules can be removed from the homogenate by contacting the proteins with support molecules (e.g., agarose beads) prior to performing the lectin affinity chromatography step. Lectin-bound proteins can be eluted from the lectin affinity matrix by any solution that effects dissociation of the protein-lectin complexes. As described in the disclosed examples, lectin-bound proteins can be eluted from the lectin affinity matrix by SDS-PAGE sample buffer. A solution that contains a sugar for which the lectin shows binding preference is also useful for eluting lectin-bound proteins from the lectin affinity matrix. For example, a solution comprising galactose in the range of from 0.04 to 0.5 M, e.g., a solution of 0.2M galactose, is useful for Outing GSIB4-binding proteins from a GSIB4 affinity matrix.

The identification, isolation or purification methods disclosed herein may further include subjecting the proteins eluted from the first lectin affinity matrix to additional steps of affinity purification using the same or different lectins. In an example of such an embodiment of the invention dial is described below, murine proteins of normal and tumor-associated tissue are purified by eluting them from a GSIB4 affinity matrix, contacting them with a Concanavalin A (Con A) chromatography matrix under conditions such that Con A-binding proteins are adsorbed to the Con A chromatography matrix, and then the Con-A-bound proteins are eluted from the ConA chromatography matrix. Alternatively, biotinylated lectin molecules that are known to bind to vascular glycoproteins, e.g., tomato lectin, can be infused into a tissue in vivo or ex vivo prior to homogenization of the tissue, and the lectin-protein complexes in the homogenate can be purified with an avidin affinity purification matrix. The lectin-binding proteins can then be eluted, and the vascular proteins can be further purified using an affinity matrix containing a different lectin that selectively binds vascular proteins, such as UEA1.

The invention further comprises identifying the lectin-binding vascular proteins isolated or purified from the tissue of interest by performing the steps of.
(f) separating and fractionating the isolated or purified proteins;
(g) digesting the isolated or purified proteins in each fraction with protease to generate peptides;
(h) purifying the peptides;
(i) determining the amino acid sequences of the peptides; and
(j) comparing the peptide amino acid sequences to known protein amino acid sequences and identifying the purified proteins.

Steps (f)-(j) are performed using known methods. For example, the isolated or purified proteins can be separated by SDS-polyacrylamide gel electrophoresis (SDS-PAGE) and fractionated by cutting the resulting gel into slices. The proteins in each fraction can be digested with trypsin in situ in the gel slices to generate peptides, and the peptides can be extracted from the gel slices. The peptides can be purified by reversed phase high pressure liquid chromatography (RP-HPLC); and the amino acid sequences of the peptides can be determined by mass spectrometry. Software and on-line services are widely available that perform the step of comparing the peptide amino acid sequences to known protein amino acid sequences to identify the purified proteins.

The methods of the present invention further enable one to identify, isolate, or purify vascular or other proteins that are specifically present in a tissue that shows an altered or abnormal pattern of growth or development due to a disease or disorder of the animal. This is done by identifying a set of lectin-binding vascular or other proteins that are present in a tissue that is induced to have an altered or abnormal pattern of growth or development by the disease or disorder as described above. Lectin-binding proteins that are present in one or more normal tissues are isolated, purified, and identified by the same methods, and the proteins that are expressed in the disease- or disorder-associated tissue but not in the normal tissue(s) are identified.

For example, the invention can be performed using tumor-associated and normal tissues from a human. As shown below, the lectin UEA1 can be used to isolate and affinity purify vascular glycoproteins directly from human tissues. Alternatively, the invention can be performed by isolating, purifying and identifying lectin-binding proteins from tumor-associated and normal tissues of a non-human mammal. Human proteins that are the homologs of vascular proteins that are specifically present in the tumor-associated tissue of the non-human mammal can then be identified by comparing the amino acid sequences of the non-human proteins to known human protein amino acid sequences.

An example of a method in which lectin-binding proteins are purified from tumor-associated and normal tissues of a mouse is described in below. The lectin used for this study is Griffonia simplicifolia IB4 (GSIB4), which is known to bind selectively to vasculature of non-primate tissues. While the vascular specificity of this lectin is well documented, the identities of GSIB4 binding proteins have not been determined. In the disclosed example, a subcutaneous human HT29 colon tumor xenograft was implanted in a SCID-bg mouse. Vascularized, tumor-associated and normal lung tissues were removed and homogenized, and immobilized GSIB4 lectin bound to agarose was used as an affinity chromatography matrix to isolate and purify GSIB4-binding proteins from the homogenates. The isolated or purified lectin-binding proteins were resolved by SDS-PAGE, digested with a protease and eluted from the gel, and mass spectrometry microsequencing (MS-MS) was used to determine the sequence of the eluted peptides. Computer programs were then used to identify the murine proteins that contain the peptides, to identify proteins that are specifically present in the HT29 tumor-associated tissue, and to identify human proteins homologous to those proteins.

Human homologs for many GSIB4-binding proteins identified from normal mouse lung are known to be expressed by endothelial or vascular smooth muscle cells. Of particular interest are proteins that are specifically present in tumor-associated tissue and contain transmembrane (TM) domains, as these proteins are generally on the cell surface and are accessible as targets of therapeutic agents, e.g., binding molecules, that bind specifically to such proteins to inhibit tumor growth or invasiveness. Three of the murine GSIB4-binding TM proteins identified by the method described in the example, integrin alpha v, TEM5, and VE-cadherin, are known to be up-regulated in tumor vasculature or VEGF-stimulated endothelial cells. Many of the murine TM proteins that were identified have human homologs that are known either to be expressed by or are specific to human vascular cells or other human tissues. Seven human TM proteins homologous to murine proteins that are specifically present in HT29 tumor-associated tissue have been identified by the methods of the invention. These proteins are:
1. CDO (CAM—related/down-regulated by oncogenes): CDO is expressed at high levels during embryogenesis in the developing nervous system, somites, dermomyotomes and myotomes but CDO expression is barely detectable in adult rat and human tissues. CDO-Fc inhibits myogenic differentiation.
2. TMEFF2 (TM protein with EGF—like and two follistatin-like domains 2): High levels of TMEFF2 mRNA are found only in brain and prostate (not placenta).
3. KIAA1484 (murine hypothetical 53.5 kDa protein (with LRR and Ig domains): Expression is reported in brain.
4. LRRN3 (LRR protein with Ig and FN3 domains): Human homolog is neuronal LRR protein 3; expression in brain is reported.
5. LRRC15 (LIB): High level expression only in brain and placenta. Low level mRNA expression detected in heart, lung liver and skeletal muscle. LRRC15 and LIB, its murine homolog, and described in more detail elsewhere herein.
6. synaptogyrin 3 (SYNGR3): SYNGR3 is a synaptic vesicle protein; expression is restricted to brain and placenta.
7. Slit-like 2 protein: involved in cellular guidance; expressed in vitro in vascular smooth muscle cells, but not in PBLs, endothelial cells, or HT29 cells; also expressed in diverse normal and tumor tissues.

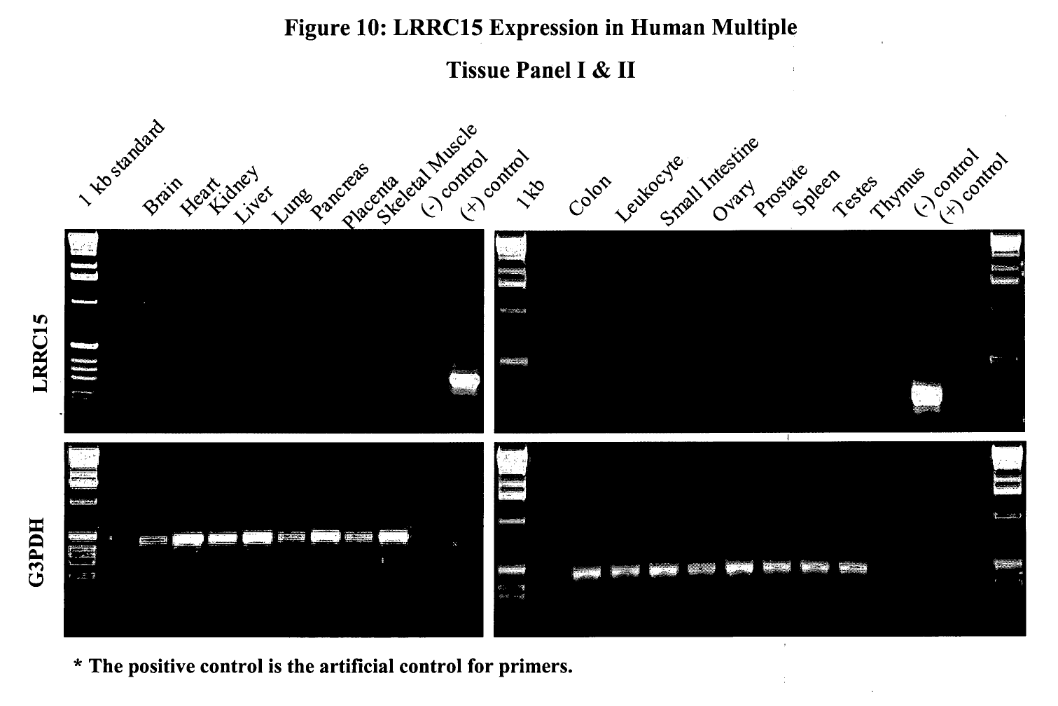

Evidence that a vascular or other protein that is present specifically in a disease- or disorder-associated tissue is a valid drug target can be obtained by using known methods to determine the specific cell types and tissue sites in which the disease- or disorder-associated proteins are expressed in non-human animals and in humans. Such methods include immunohistochemistry (IHC), in situ nucleic acid hybridization (ISH), and reverse transcription-polymerase chain reaction (RT-PCR), as described in more detail in the examples below. In the disclosed examples, the expression of two human genes encoding TM proteins homologous to murine tumor-associated proteins (LRRC15 and the Slit-like 2 protein) in various cell types in vitro. and in diverse normal and tumor tissues was detected using RT-PCR (see FIGS. 10 through 17).

### III. LRRC15

In certain embodiments, the present invention is directed to methods of treating or diagnosing hyperproliferative diseases such as cancer, comprising the use of binding molecules which specifically bind to LRRC15. Leucine-rich repeats (LRRs) are 20-29-residue sequence motifs present in a number of proteins with diverse functions such as hormone-receptor interactions, enzyme inhibition, cell adhesion and cellular trafficking. The primary function of these motifs appears to be to provide a versatile structural framework for the formation of protein-protein interactions. A number of recent studies revealed the involvement of LRR proteins in early mammalian development, neural development, cell polarization, regulation of gene expression and apoptosis signaling. In addition, LRRs may be critical to the morphology and dynamics of cytoskeleton. See, e.g., Kobe B., and Kajava A. V., Curr. Opin. Struct. Biol. 11: 725-732 (2001), which is incorporated herein by reference in its entirety.

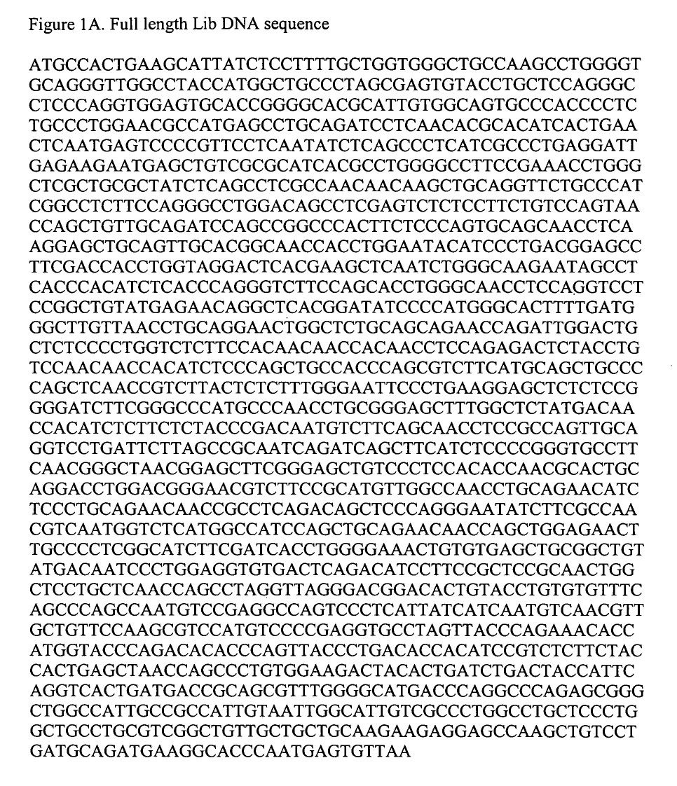

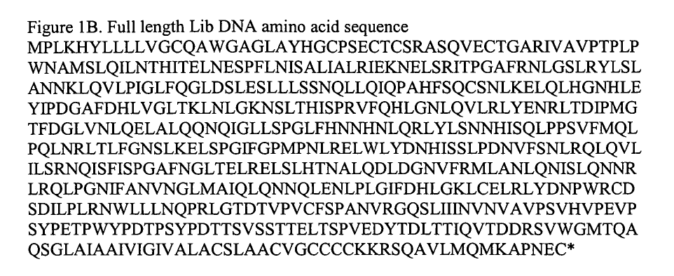

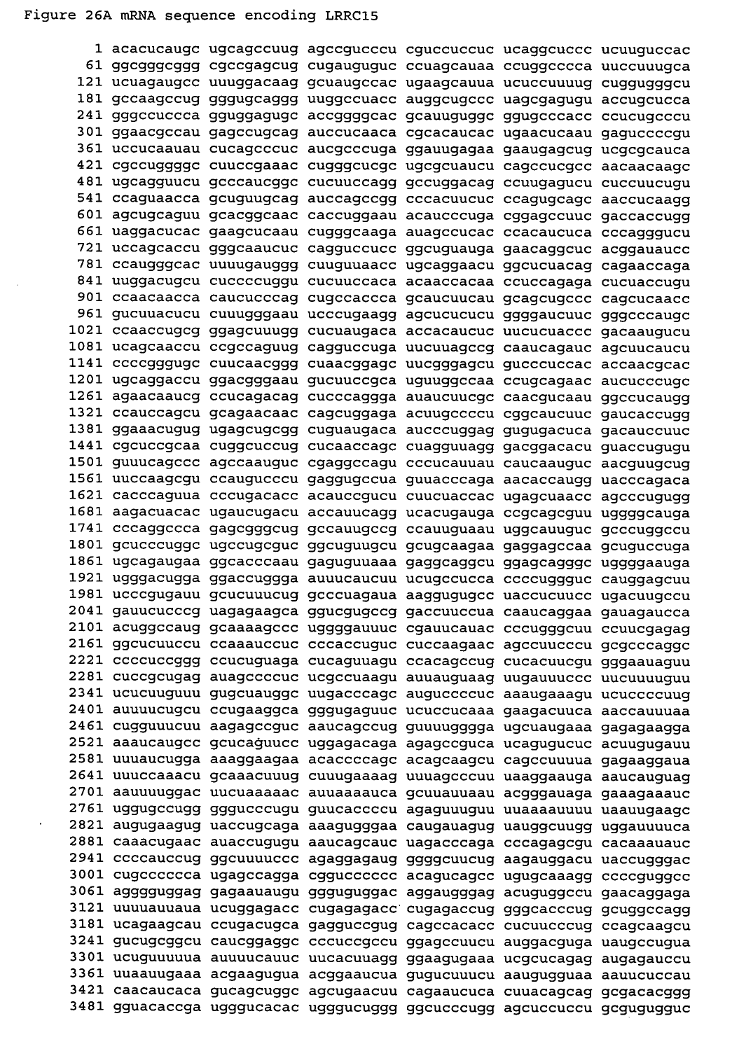

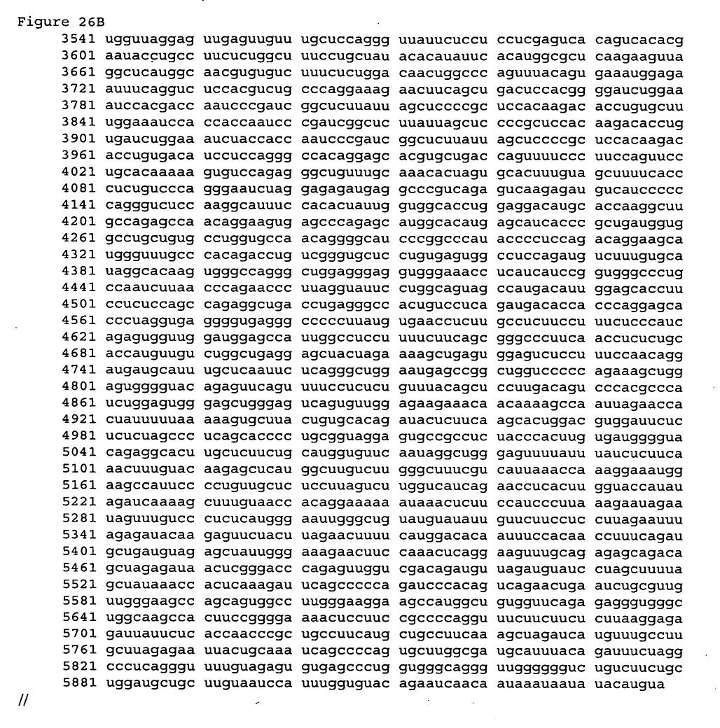

LRRC15 is the human homolog of the murine LIB protein identified by GSIB4 affinity as described in the examples. LRRC15 is a leucine-rich transmembrane protein of 581 amino acids. See, e.g., Genbank Accession Number Q8TF66. The amino acid sequence of LRRC15 is designated herein as SEQ ID NO:2 and is shown in FIG. 1B. The nucleic acid coding region encoding LRRC15 is designated herein as SEQ ID NO:1 and is shown in FIG. 1A. The full cDNA sequence of 5938 bases is available as Genbank Accession Number BK001325. The LRRC15 mRNA sequence is shown as FIG. 26, and is designated herein as SEQ ID NO:5. See also, Reynolds, P. A., et al., Genes Dev. 17:2094-2107 (2003), and Satoh, K., et al., Biochem. Biophys. Res. Commun. 290:756-762 (2002).

The LRRC15 mRNA comprises 143 bases of 5′ untranslated region extending from nucleotide 1 to nucleotide 145 of SEQ ID NO:5. The coding region of LRRC15 extends from nucleotide 144 to nucletoide 1889 of SEQ ID NO:5. The 3′ untranslated region extends from nucleotide 1890 to nucleotide 5938 of SEQ ID NO:5. It will be understood by those of ordinary skill in the art that transcripts encoding LRRC15 may vary for a variety of reasons, e.g., differential splicing of genomic DNA or the length of the poly-A tail added to the end of the transcript. Thus, it will be well understood that the mRNA designated herein as SEQ ID NO:5 is an example of an mRNA encoding LRRC15, and is not limiting.

The LRRC15 translation product comprises, consists essentially of, or consists of an N-terminal signal peptide extending from amino acid 1 to about amino acid 21 of SEQ ID NO:2, and a mature protein extending from about amino acid 22 to amino acid 581 of SEQ ID NO:2. LRRC15 comprises, consists essentially of, or consists of an extracellular domain extending from about amino acid 22 to about amino acid 538 of SEQ ID NO:2, the extracellular domain containing 15 leucine-rich repeats extending from about amino acid 51 to about amino acid 75 of SEQ ID NO:2 (LRR1); from about amino acid 76 to about amino acid 99 of SEQ ID NO:2 (LRR2); from about amino acid 100 to about amino acid 123 of SEQ ID NO:2 (LRR3); from about amino acid 125 to about amino acid 147 of SEQ ID NO:2 (LRR4); from about amino acid 148 to about amino acid 171 of SEQ ID NO:2 (LRR5); from about amino acid 173 to about amino acid 195 of SEQ ID NO:2 (LRR6); from about amino acid 196 to about amino acid 219 of SEQ ID NO:2 (LRR7); from about amino acid 221 to about amino acid 243 of SEQ ID NO:2 (LRR8); from about amino acid 244 to about amino acid 267 of SEQ ID NO:2 (LRR9); from about amino acid 269 to about amino acid 291 of SEQ ID NO:2 (LRR10); from about amino acid 292 to about amino acid 315 of SEQ ID NO:2 (LRR11); from about amino acid 317 to about amino acid 339 of SEQ ID NO:2 (LRR12); from about amino acid 340 to about amino acid 363 of SEQ ID NO:2 (LRR13); from about amino acid 364 to about amino acid 387 of SEQ ID NO:2 (LRR14); or from about amino acid 389 to about amino acid 411 of SEQ ID NO:2 (LRR15). LRRC15 comprises, consists essentially of, or consists of a transmembrane domain extending from about amino acid 539 to about amino acid 559 of SEQ ID NO:2, and a cytoplasmic domain extending from about amino acid 560 to amino acid 581 of SEQ ID NO:2.

An immunogenic fragment of LRRC15 comprises, consists essentially of, or consists of a peptide extending from about amino acid 127 of SEQ ID NO:2 to about amino acid 157 of SEQ ID NO:2 (amino acids 2 to 32 of SEQ ID NO:21). An additional immunogenic fragment of LRRC15 comprises, consists essentially of, or consists of a peptide extending from about amino acid 480 of SEQ ID NO:2 to about amino acid 509 of SEQ ID NO:2 (amino acids 3 to 32 of SEQ ID NO:22). Additional immunogenic fragments, as predicted by the Kyte and Doolittle hydropathy algorithm (Kyte, J. and Doolittle, R.,. J. Mol. Biol. 157: 105-132 (1982)) comprise, consist essentially of, or consist of from about amino acid 25 of SEQ ID NO:2 to about amino acid 60 of SEQ ID NO:2; from about amino acid 85 of SEQ ID NO:2 to about amino acid 95 of SEQ ID NO:2; from about amino acid 140 of SEQ ID NO:2 to about amino acid 170 of SEQ ID NO:2; from about amino acid 200 of SEQ ID NO:2 to about amino acid 215 of SEQ ID NO:2; from about amino acid 240 of SEQ ID NO:2 to about amino acid 255 of SEQ ID NO:2; from about amino acid 365 of SEQ ID NO:2 to about amino acid 380 of SEQ ID NO:2; from about amino acid 420 of SEQ ID NO:2 to about amino acid 430 of SEQ ID NO:2; and from about amino acid 480 of SEQ ID NO:2 to about amino acid 505 of SEQ ID NO:2. Additional immunogenic fragments, as predicted using the Goldman, Engelman and Steitz Transbilayer Helices Prediction algorithm (Engelman, D. M. et al. Annu. Rev. Biophys. Biophys. Chem. 15:321-353 (1986)) comprise, consist essentially of, or consist of from about amino acid 25 of SEQ ID NO:2 to about amino acid 50 of SEQ ID NO:2; from about amino acid 75 of SEQ ID NO:2 to about amino acid 115 of SEQ ID NO:2; from about amino acid of SEQ ID NO:2145 to about amino acid 175 of SEQ ID NO:2; from about amino acid 195 of SEQ ID NO:2 to about amino acid 215 of SEQ ID NO:2; from about amino acid 225 of SEQ ID NO:2 to about amino acid 270 of SEQ ID NO:2; from about amino acid 285 of SEQ ID NO:2 to about amino acid 325 of SEQ ID NO:2; from about amino acid 326 of SEQ ID NO:2 to about amino acid 385 of SEQ ID NO:2; from about amino acid 386 of SEQ ID NO:2 to about amino acid 450 of SEQ ID NO:2; and from about amino acid 475 of SEQ ID NO:2 to about amino acid 540 of SEQ ID NO:2.

In the context of the amino acids comprising the various structural and functional domains of an LRRC15 polypeptide, the term “about” includes the particularly recited value and values larger or smaller by several (e.g., 10, 9, 8, 7, 6, 5, 4, 3, 2, or 1) amino acids. Since the location of these domains have been predicted by computer graphics, one of ordinary skill would appreciate that the amino acid residues constituting these domains may vary slightly (e.g., by about 1 to 15 residues) depending on the criteria used to define the domain. Thus in various embodiments, the extracellular domain of LRRC15 comprises, consists essentially of, or consists of, for example, amino acids 19 to 538 of SEQ ID NO:2, amino acids 29 to 538 of SEQ ID NO:2, amino acids 21 to 538 of SEQ ID NO:2, amino acids 22 to 538 of SEQ ID NO:2, amino acids 23 to 538 of SEQ ID NO:2, amino acids 24 to 538 of SEQ ID NO:2, amino acids 25 to 538 of SEQ ID NO:2, amino acids 22 to 535 of SEQ ID NO:2, amino acids 22 to 536 of SEQ ID NO:2, amino acids 22 to 537 of SEQ ID NO:2, amino acids 22 to 538 of SEQ ID NO:2, amino acids 22 to 539 of SEQ ID NO:2, amino acids 22 to 540 of SEQ ID NO:2, or amino acids 22 to 541 of SEQ ID NO:2. In additional embodiments, the extracellular domain of LRRC15 comprises, consists essentially of, or consists of, for example, amino acids x to y, where x is any of amino acids 12, 13, 14, 15, 16, 17, 18, 19, 20, 21, 22, 23, 24, 25, 26, 27, 28, 29, 30, 31, or 32 of SEQ ID NO:2, and where y is any of amino acids 528, 539, 530, 531, 532, 533, 534, 535, 536, 537, 538, 539, 540, 541, 542, 543, 544, 545, 546, 547, or 548 of SEQ ID NO:2. LRRC15 if further predicted comprise potential N-linked glycosylation sites at amino acid residues 75 and 369.

Reynolds et al., noted that LRRC15 is normally expressed on the leading edge of invasive cells in the placental cytotrophoblast layer, but showed that its expression was dramatically upregulated by a transcription factor expressed in desmoplastic small round cell tumor cells. Reynolds et al. further demonstrated that siRNA-mediated suppression of LRRC15 expression in breast cancer cells lead to abrogation of invasiveness in an in vitro system, hypothesizing that LRRC15 may be generally involved in tumor invasiveness. Expression of the rat homolog of LRRC15, designated LIB, was induced in rat astrocytes in response to beta-amyloid protein. See Satoh et al.

As is shown in more detail in the examples, the present inventors have demonstrated expression of LRRC15 in placenta, as well as in several normal human cell lines and at least one normal lung tissue sample. The inventors further found LRRC expression in various breast, colon, and lung tumor cell lines and in various human tumor tissues recovered from ovary, breast, lung, colon, and prostate.

IV. Treatment Methods Using Therapeutic Binding Molecules, in Particular, LRRC15—Specific Antibodies, or Immunospecific Fragments Thereof

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of a binding molecule, more specifically a binding polypeptide, and even more specifically an antibody or immunospecific fragment thereof, that binds to a tumor- or tumor-associated protein identified by the methods described herein. A specific embodiment of the present invention is a method of treatment as above, where the binding molecule binds specifically to at least one epitope of LRRC15.

A therapeutic binding molecule, e.g., a binding polypeptide, e.g., an antibody that binds specifically to a disease- or disorder-associated protein identified according to methods described herein, to be used in treatment methods disclosed herein can be prepared and used as a therapeutic agent that stopes, reduces, prevents, or inhibits cellular activities involved in cellular hyperproliferation, e.g., cellular activities that induce the altered or abnormal pattern of vascularization that is often associated with hyperproliferative diseases or disorders. Characteristics of proteins that are suitable targets for such binding molecules include location on the cell surface and disease- or disorder-specific expression; e.g., by cells of tumor-induced or inflammatory vascular tissue. Therapeutic binding molecules that bind specifically to such disease- or disorder-associated proteins are referred to herein as binding molecules or binding polypeptides. In certain embodiments, the binding molecule has at least one binding domain which specifically binds to a target molecule such as a polypeptide, e.g., a tumor-expressed or tumor-associated cell surface antigen. A preferred target for such binding molecules is LRRC15.

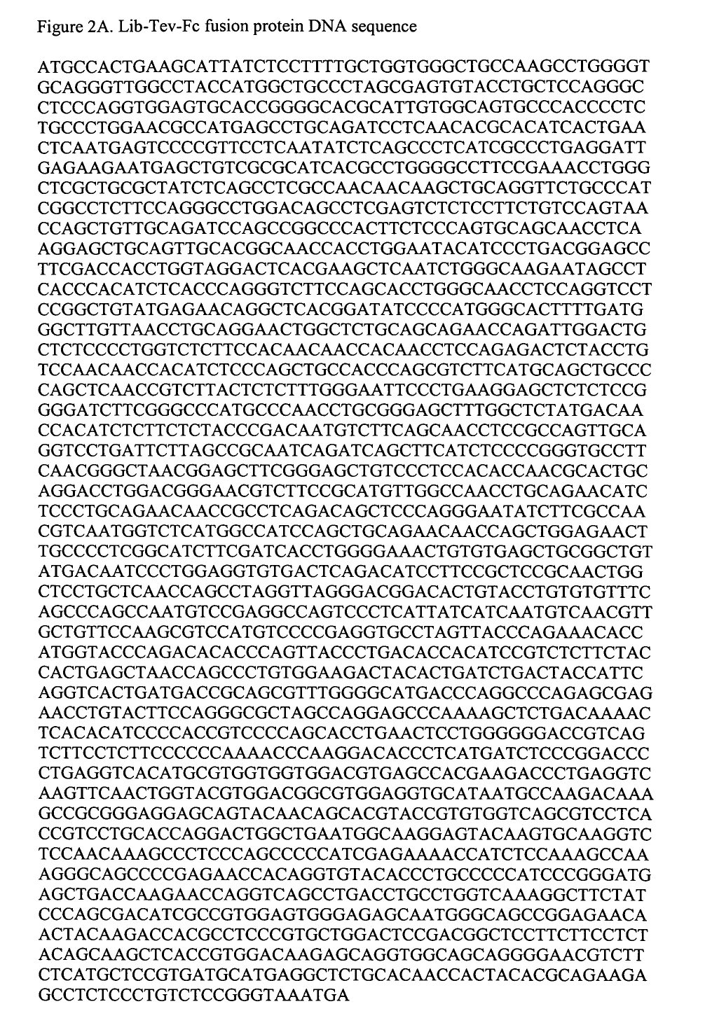

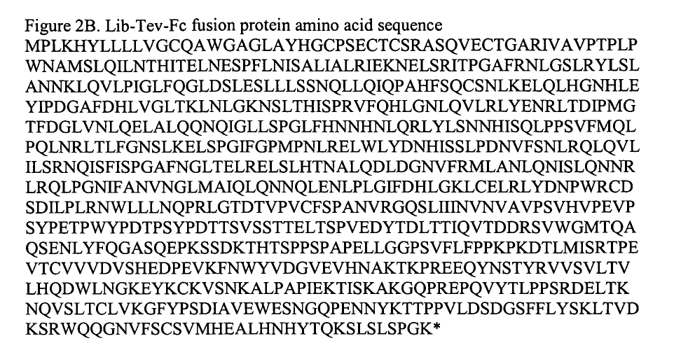

Binding polypeptides include antibodies or immunospecific fragments thereof such as monoclonal, chimeric or humanized antibodies, and fragments of antibodies that bind specifically to tumor-associated proteins such as LRRC15. The antibodies may be monovalent, bivalent, polyvalent, or bifunctional antibodies, and the antibody fragments include Fab F(ab′)₂, and Fv. Therapeutic binding molecules produced according to the invention also include fusion proteins that target a ligand or receptor of a disease- or disorder-associated protein isolated, purified, and/or identified by the methods described herein. Another type of binding polypeptide, also used herein as an immunogen, comprises a non-antigen-specific fragment of an immunoglobulin joined to the extracellular domain of a transmembrane disease- or disorder-associated protein, e.g., about amino acids 22 to 537 or 538 of LRRC15, to generate a receptor:Ig fusion protein that antagonizes and neutralizes the cellular function of the target protein. An exemplary fusion comprising amino acids 1 to 537 of SEQ ID NO:2 fused to an IgG1 Fc region is shown in FIG. 2B and is designated herein as SEQ ID NO:4. The nucleotide sequence encoding this fusion protein is shown in FIG. 2A and is designated herein as SEQ ID NO:3.

Therapeutic binding molecules according to the invention can be used in unlabeled or unconjugated form, or can be coupled or linked to cytotoxic moieties such as radiolabels and biochemical cytotoxins to produce agents that exert therapeutic effects.

In certain embodiments, a binding domain on a binding molecule or binding polypeptide is an antigen binding domain, and the binding polypeptide is an antibody, or immunospecific fragment thereof. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

While a lectin-binding protein that is identified according to the methods of the invention can be expressed in disease- or disorder-associated tissue, the therapeutic agent that binds the targeted protein can also exert a therapeutic effect by binding to the targeted protein present on non-vascular tissues associated with the disease or disorder. For example, the invention includes methods for inhibiting tumor angiogenesis and growth in a mammal comprising administering a binding agent that binds specifically to a vascular protein identified by the invention as being specifically present in tumor-associated tissue. The vascular protein identified by the invention as being specifically present in tumor-associated tissue may also be expressed by the tumor tissue itself, so that in addition to inhibiting tumor angiogenesis through binding to the targeted protein in the tumor vasculature, an anti-tumor agent, e.g., a binding molecule, that binds specifically to the targeted protein according to the invention might also inhibit growth of a tumor by binding to and killing tumor cells directly, or by blocking invasiveness of tumor cells.

The present invention provides methods for treating various hyperproliferative disorders, e.g., by inhibiting tumor growth, in a mammal, comprising, consisting essentially of, or consisting of administering to the mammal an effective amount of a binding agent that binds specifically to a transmembrane vascular protein identified by the invention as being specifically or predominantly present in tumor cells or tumor-associated tissue. Included in the invention are methods comprising administering a binding agent that binds specifically to a protein selected from the group consisting of CDO, TMEFF2, KIAA1484, LRRN3, LRRC15, synaptogyrin 3, and Slit-like 2 protein. Also included in the invention are methods comprising, consisting essentially of, or consisting of administering a binding agent that comprises, consists essentially of, or consists of an extracellular domain of a protein selected from the group consisting of CDO, TMEFF2, KIAA1484, LRRN3, LRRC15, synaptogyrin 3, and Slit-like 2 protein.

In addition to antibodies and immunospecific fragments thereof, binding molecules of the present invention include a fusion protein, an agent which elicits a T-cell response specific for CDO, TMEFF2, KIAA1484, LRRN3, LRRC15, synaptogyrin 3, or Slit-like 2 protein, a cytokine, a soluble version of CDO, TMEFF2, KIAA1484, LRRN3, LRRC15, synaptogyrin 3, or Slit-like 2 protein, a cellular matrix component, a lectin, an antisense RNA, an siRNA, a ribozyme, and a small molecule. Similar binding molecules may be used in the in vitro and in vivo diagnostic methods described in more detail below.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of a binding agent, e.g., a binding molecule, more specifically a binding polypeptide, and even more specifically an antibody or immunospecific fragment thereof, which specifically binds to one or more epitopes of LRRC15.

In particular, the present invention includes a method for treating a hyperproliferative disease, e.g., inhibiting tumor formation, tumor growth, tumor invasiveness, and/or metastasis formation in an animal, e.g., a mammal, e.g., a human patient, or prolonging survival of the animal, where the method comprises, consists essentially of, or consists of administering to an animal in need of such treatment an effective amount of a composition comprising, consisting essentially of, or consisting of, in addition to a pharmaceutically acceptable carrier, a binding molecule which specifically binds to an LRRC15 gene product. Such gene products include, but are not limited to, an LRRC15 polypeptide comprising, consisting essentially of, or consisting of amino acids 1 to 581 of SEQ ID NO:2 or a fragment thereof, an LRRC15 variant polypeptide at least 70%, 75%, 80%, 85%, 90%, or 95% identical to amino acids 1 to 581 or SEQ ID NO:2 or a fragment thereof, an LRRC15 variant polypeptide at least 70%, 75%, 80%, 85%, 90%, or 95% identical to an LRRC15 polypeptide fragment comprising, consisting essentially of, or consisting of amino acids 22 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 22 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 22 to 538 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 538 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 538 of SEQ ID NO:2, and an LRRC15 messenger RNA comprising, consisting essentially of, or consisting of nucleotides 1 to 5938 of SEQ ID NO:5 or a fragment thereof.

In the above embodiments, exemplary “fragments” of an LRRC15 polypeptide consisting of amino acids 1 to 581 of SEQ ID NO:2 include, but are not limited to: a fragment comprising, consisting essentially of, or consisting of amino acids 1 to 538 of SEQ ID NO:2, a fragment comprising, consisting essentially of, or consisting of amino acids 1 to 537 of SEQ ID NO:2, a fragment comprising, consisting essentially of, or consisting of amino acids 2 to 581 of SEQ ID NO:2, a fragment comprising, consisting essentially of, or consisting of amino acids 2 to 538 of SEQ ID NO:2, a fragment comprising, consisting essentially of, or consisting of amino acids 2 to 537 of SEQ ID NO:2, a fragment comprising, consisting essentially of, or consisting of amino acids 22 to 581 of SEQ ID NO:2, a fragment comprising, consisting essentially of, or consisting of amino acids 22 to 538 of SEQ ID NO:2, a fragment comprising, consisting essentially of, or consisting of amino acids 22 to 537 of SEQ ID NO:2, a fragment comprising, consisting essentially of, or consisting of amino acids 127 to 157 of SEQ ID NO:2, and a fragment comprising, consisting essentially of, or consisting of amino acids 480 to 509 of SEQ ID NO:2. Corresponding fragments of a variant LRRC15 polypeptide at least 70%, 75%, 80%, 85%, 90%, or 95% identical to amino acids 1 to 581 of SEQ ID NO:2 are also contemplated.

In the above embodiments, exemplary “fragments” of an LRRC15 polypeptide consisting of amino acids 1 to 581 of SEQ ID NO:2 include, but are not limited to: a fragment comprising, consisting essentially of, or consisting of from about amino acid 25 of SEQ ID NO:2 to about amino acid 60 of SEQ ID NO:2; from about amino acid 85 of SEQ ID NO:2 to about amino acid 95 of SEQ ID NO:2; from about amino acid 140 of SEQ ID NO:2 to about amino acid 170 of SEQ ID NO:2; from about amino acid 200 of SEQ ID NO:2 to about amino acid 215 of SEQ ID NO:2; from about amino acid 240 of SEQ ID NO:2 to about amino acid 255 of SEQ ID NO:2; from about amino acid 365 of SEQ ID NO:2 to about amino acid 380 of SEQ BD NO:2; from about amino acid 420 of SEQ ID NO:2 to about amino acid 430 of SEQ ID NO:2; and from about amino acid 480 of SEQ ID NO:2 to about amino acid 505 of SEQ ID NO:2; from about amino acid 25 of SEQ ID NO:2 to about amino acid 50 of SEQ ID NO:2; from about amino acid 75 of SEQ ID NO:2 to about amino acid 115 of SEQ ID NO:2; from about amino acid of SEQ ID NO:2145 to about amino acid 175 of SEQ ID NO:2; from about amino acid 195 of SEQ BD NO:2 to about amino acid 215 of SEQ ID NO:2; from about amino acid 225 of SEQ ID NO:2 to about amino acid 270 of SEQ ID NO:2; from about amino acid 285 of SEQ ID NO:2 to about amino acid 325 of SEQ ID NO:2; from about amino acid 326 of SEQ ID NO:2 to about amino acid 385 of SEQ ID NO:2; from about amino acid 386 of SEQ ID NO:2 to about amino acid 450 of SEQ ID NO:2; and from about amino acid 475 of SEQ ID NO:2 to about amino acid 540 of SEQ ID NO:2.

Additional exemplary “fragments” of an LRRC15 peptide consisting of amino acids 1 to 581 of SEQ ID NO:2 include, but are not limited to those fragments comprising, consisting essentially of, or consisting of one or more leucine-rich-repeat (LRR) regions of LRRC15. Such fragments, include, for example, a fragment comprising, consisting essentially of, or consisting of amino acids 51 to 75 of SEQ ID NO:2 (LRR1); a fragment comprising, consisting essentially of, or consisting of amino acids 76 to 99 of SEQ ID NO:2 (LRR2); a fragment comprising, consisting essentially of, or consisting of amino acids 100 to 123 of SEQ ID NO:2 (LRR3); a fragment comprising, consisting essentially of, or consisting of amino acids 125 to 147 of SEQ ID NO:2 (LRR4); a fragment comprising, consisting essentially of, or consisting of amino acids 148 to 171 of SEQ ID NO:2 (LRR5); a fragment comprising, consisting essentially of, or consisting of amino acids 173 to 195 of SEQ ID NO:2 (LRR6); a fragment comprising, consisting essentially of, or consisting of amino acids 196 to 219 of SEQ ID NO:2 (LRR7); a fragment comprising, consisting essentially of, or consisting of amino acids 221 to 243 of SEQ ID NO:2 (LRR8); a fragment comprising, consisting essentially of, or consisting of amino acids 244 to 267 of SEQ ID NO:2 (LRR9); a fragment comprising, consisting essentially of, or consisting of amino acids 269 to 291 of SEQ ID NO:2 (LRR10); a fragment comprising, consisting essentially of, or consisting of amino acids 292 to 315 of SEQ ID NO:2 (LRR11); a fragment comprising, consisting essentially of, or consisting of amino acids 317 to 339 of SEQ ID NO:2 (LRR12); a fragment comprising, consisting essentially of, or consisting of amino acids 340 to 363 of SEQ ID NO:2 (LRR13); a fragment comprising, consisting essentially of, or consisting of amino acids 364 to 387 of SEQ ID NO:2 (LRR14); and a fragment comprising, consisting essentially of, or consisting of amino acids 389 to 411 of SEQ ID NO:2 (LRR15). Corresponding fragments of a variant LRRC15 polypeptide at least 70%, 75%, 80%, 85%, 90%, or 95% identical to amino acids 1 to 581 of SEQ ID NO:2 are also contemplated.

Additional exemplary “fragments” of an LRRC15 peptide consisting of amino acids 1 to 581 of SEQ ID NO:2 include, but are not limited to those fragments comprising, consisting essentially of, or consisting of amino acids x to y, where x is any of amino acids 12, 13, 14, 15, 16, 17, 18, 19, 20, 21, 22, 23, 24, 25, 26, 27, 28, 29, 30, 31, or 32 of SEQ ID NO:2, and where y is any of amino acids 528, 539, 530, 531, 532, 533, 534, 535, 536, 537, 538, 539, 540, 541, 542, 543, 544, 545, 546, 547, or 548 of SEQ ID NO:2. Corresponding fragments of a variant LRRC15 polypeptide at least 70%, 75%, 80%, 85%, 90%, or 95% identical to amino acids 1 to 581 of SEQ ID NO:2 are also contemplated.

As known in the art, “sequence identity” between two polypeptides is determined by comparing the amino acid sequence of one polypeptide to the sequence of a second polypeptide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

Exemplary “fragments” of an LRRC15 messenger RNA comprising, consisting essentially of, or consisting of nucleotides 1 to 5938 of SEQ ID NO:5 include, but are not limited to those fragments comprising, consisting essentially of, or consisting of nucleotides 1 to 143 of SEQ ID NO:5, nucleotides 144 to 1889 of SEQ ID NO:5 and nucleotides 1890 to 5938 of SEQ ID NO:5. Additional fragments include, but are not limited to fragments which comprise, consist essentially of, or consist of at least 5, and least 10, at least 25, at least 50, at least 100, at least 250 at least 500, at least 750 at least 1000, at least 2000, at least 3000, at least 4000, or at least 5000 nucleotides of SEQ ID NO:5. Such nucleotides may be contiguous, i.e., in a linear sequence, or non-contiguous, i.e., from at least two different regions of a nucleic acid brought together by, e.g., secondary structure.

By “a binding molecule which specifically binds to an LRRC messenger RNA comprising, consisting essentially of, or consisting of nucleotides 1 to 5938 of SEQ ID NO:5 or a fragment thereof” is meant any molecule that binds to such a nucleic acid, and includes nucleic acid binding molecules, polypeptide bindign molecules, and small molecule binding molecules. Nucleic acid binding molecules typically bind to a LRRC15 mRNA or fragment thereof as described above by hybridization. Such nucleic acid binding molecules include, but are not limited to antisense nucleic acids, siRNA constructs, and ribozymes, e.g., hammerhead ribozymes. Such binding molecules typically hybridize to their complementary nucleic acids under standard hybridiztion conditions as described herein.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of LRRC15, where the epitope comprises, consists essentially of, or consists of at least about four to five amino acids amino acids of SEQ ID NO:2, at least seven, at least nine, or between at least about 15 to about 30 amino acids of SEQ ID NO:2. The amino acids of a given epitope of SEQ ID NO:2 as described may be, but need not be contiguous. In certain embodiments, the at least one epitope of LRRC15 comprises, consists essentially of, or consists of a non-linear epitope formed by the extracellular domain of LRRC15 as expressed on the surface of a cell. Thus, in certain embodiments the at least one epitope of LRRC15 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of LRRC15, where the epitope comprises, consists essentially of, or consists of, in addition to one, two, three, four, five, six or more contiguous or non-contiguous amino acids of SEQ ID NO:2 as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of an LRRC15-specific antibody or immunospecific fragment thereof, and a pharmaceutically acceptable carrier. Types of cancer to be treated include, but are not limited to, colon cancer, lung cancer, breast cancer, pancreatic cancer, and prostate cancer.

A binding molecule for use in the present invention is typically a binding polypeptide, in particular an antibody or immunospecific fragment thereof. In certain embodiments, an antibody or fragment thereof binds specifically to at least one epitope of LRRC15 or fragment or variant described above, i.e., binds to such an epitope more readily than it would bind to an unrelated, or random epitope; binds preferentially to at least one epitope of LRRC15 or fragment or variant described above, i.e., binds to such an epitope more readily than it would bind to a related, similar, homologous, or analogous epitope; competitively inhibits binding of a reference antibody which itself binds specifically or preferentially to a certain epitope of LRRC15 or fragment or variant described above; or binds to at least one epitope of LRRC15 or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻³ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴M, about 10⁻¹⁴ M, about 5×10⁻¹⁵ M, or about 10⁻¹⁵ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻²M” might include, for example, from 0.05 M to 0.005 M.

In specific embodiments, binding molecules, e.g., antibodies or immunospecific fragments thereof for use in the diagnostic and treatment methods disclosed herein bind LRRC15 polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. More preferably, binding molecules, e.g., antibodies or immunospecific fragments thereof for use in the diagnostic and treatment methods disclosed herein bind LRRC15 polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, binding molecules, e.g., antibodies or immunospecific fragments thereof for use in the diagnostic and treatment methods disclosed herein bind LRRC15 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, binding molecules, e.g., antibodies or immunospecific fragments thereof for use in the diagnostic and treatment methods disclosed herein bind LRRC15 polypeptides or fragments or variants thereof with an on rate (k(on)) greater than or equal to 10⁵ M⁻¹ sec⁻¹, 5×10⁻⁵ M⁻¹ sec⁻¹, 10⁶ M⁻¹ sec⁻¹, or 5×106 M⁻¹ sec⁻¹ or 10⁷ M⁻¹ sec⁻¹.

In various embodiments, one or more binding molecules as described above is an antagonist of LRRC15 activity. For example binding of the binding molecule to LRRC15, as expressed in tumor-associated vascular tissue, blocks angiogenesis in the tissue thereby inhibiting tumor growth or spread. Alternatively, binding of the binding molecule to LRRC15, as expressed on a tumor cell may inhibit the ability of LRRC15 to facilitate invasiveness of the tumor cell, e.g., through prevention or retardation of metastatic growth, or through prevention or retardation of tumor spread to adjacent tissues. In addition, binding of the binding molecule to LRRC15, as expressed on a tumor cell may facilitate killing of the tumor cell, for example through effector functions such as complement-mediated lysis or antibody-dependent cellular cytotoxicity.

V. Diagnostic or Prognostic Methods Using LRRC15-Specific Binding Molecules and Nucleic Acid Amplification Assays

LRRC15-specific binding molecules, e.g., antibodies, or fragments, derivatives, or analogs thereof, can be used for diagnostic purposes to detect, diagnose, or monitor diseases, disorders, and/or conditions associated with the aberrant expression and/or activity of LRRC15. While LRRC15 expression is generally limited to certain tissues in the placenta, the inventors have detected significant levels of LRRC15 expression in a number of tumor cells and tumor-associated tissues, e.g., in breast, lung, colon, ovary, pancreas, and prostate tumor-associated tissues.

LRRC15-specific binding molecules disclosed herein, e.g., antibodies or fragments thereof, are useful for diagnosis, treatment, prevention and/or prognosis of hyperproliferative disorders in mammals, preferably humans. Such disorders include, but are not limited to, cancer, neoplasms, tumors and/or as described under elsewhere herein, especially LRRC15-associated cancers such as breast, ovarian, lung, prostate, pancreatic, and colon cancers.

In particular, it is believed that certain tumor-associated tissues in express significantly enhanced levels of LRRC15 protein and mRNA when compared to corresponding “standard” levels. Further, it is believed that enhanced LRRC15 expression can be detected in certain body fluids (e.g., sera, plasma, urine, and spinal fluid) or cells or tissue from mammals with such a cancer when compared to cognate samples from mammals, e.g., humans, of the same species not having the cancer.

For example, as disclosed herein, LRRC15 expression is associated with at least breast, lung, ovarian, prostate, and colon tumor tissues. Accordingly, binding molecules, e.g., antibodies (and antibody fragments) directed against LRRC15 may be used to detect particular tissues expressing LRRC15. These diagnostic assays may be performed in vivo or in vitro, such as, for example, on blood samples, biopsy tissue or autopsy tissue.

Thus, the invention provides a diagnostic method useful during diagnosis of a cancers and other hyperproliferative disorders, which involves measuring the expression level of LRRC15 protein or transcript in tissue or other cells or body fluid from an individual and comparing the measured expression level with a standard LRRC15 expression levels in normal tissue or body fluid, whereby an increase in the expression level compared to the standard is indicative of a disorder.

One embodiment provides a method of detecting the presence of abnormal hyperproliferative cells, e.g., precancerous or cancerous cells, in a fluid or tissue sample, comprising assaying for the expression, e.g., transcription or translation, of LRRC15 in tissue or body fluid samples of an individual and comparing the presence or level of LRRC15 expression in the sample with the presence or level of LRRC15 expression in a panel of standard tissue or body fluid samples, where detection of LRRC15 expression or an increase in LRRC15 expression over the standards is indicative of aberrant hyperproliferative cell growth.

More specifically, the present invention provides a method of detecting the presence of abnormal hyperproliferative cells in a body fluid or tissue sample, comprising (a) assaying for the expression of LRRC15 in tissue or body fluid samples of an individual using one or more binding molecules, e.g., binding polypeptides, e.g., LRRC15-specific antibodies or immunospecific fragments thereof which specifically bind to at least one epitope of LRRC15, and (b) comparing the presence or level of LRRC15 expression in the sample with a the presence or level of LRRC15 expression in a panel of standard tissue or body fluid samples, whereby detection of LRRC15 expression or an increase in LRRC15 expression over the standards is indicative of aberrant hyperprloiferative cell growth.

In addition, the present invention provides a method of detecting the presence of abnormal hyperproliferative cells in a body fluid or tissue sample, comprising (a) assaying for the transcription of LRRC15 in messenger RNA (mRNA) extracted from tissue or body fluid samples of an individual using one or more nucleic acid primers or probes, e.g., oligonucleotides capable of specifically binding to and/or amplifying a portion of LRRC15 mRNA, and (b) comparing the presence or level of LRRC15 mRNA expression in the sample with a the presence or level of LRRC15 expression in a panel of standard tissue or body fluid mRNA samples, whereby detection of LRRC15 mRNA expression or an increase in LRRC15 expression over the standards is indicative of aberrant hyperprloiferative cell growth.

Messenger RNA detection assays, e.g., amplification assays, e.g., reverse transcriptase polymerase chain reaction (RT-PCR) are well known to those of ordinary skill in the art. Suitable oligonucleotides for use in such diagnostic methods, e.g., SEQ ID NOs 6 and 7, are described in the Examples.

With respect to cancer, the presence of a relatively high amount of LRRC15 protein or transcript in biopsied tissue from an individual may indicate the presense of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

LRRC15-specific binding molecules can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of LRRC15 in an animal, preferably a mammal and most preferably a human. In one embodiment, diagnosis comprises: a) administering (for example, parenterally, subcutaneously, or intraperitoneally) to a subject an effective amount of a labeled binding molecule, e.g., an antibody or fragment thereof, which specifically binds to LRRC15; b) waiting for a time interval following the administering for permitting the labeled binding molecule to preferentially concentrate at sites in the subject where LRRC15 is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of LRRC15. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of LRRC15 expression include those detectable by X-radiography, nuclear magnetic resonance immaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR. include those with a detectable characteristic spin, such as deuterium, which may be incorporated into the antibody by labeling of nutrients for the relevant hybridoma. Where in vivo imaging is used to detect enhanced levels of LRRC15 expression for diagnosis in humans, it may be preferable to use human antibodies or “humanized” chimeric monoclonal antibodies. Such antibodies can be produced using techniques described herein or otherwise known in the art. For example methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibiting enhanced LRRC15 expression will experience a worse clinical outcome relative to patients whose expression level decreases nearer the standard level.

By “assaying the expression level of the tumor associated LRRC15 polypeptide” is intended qualitatively or quantitatively measuring or estimating the level of LRRC15 polypeptide in a first biological sample either directly (e.g., by determining or estimating absolute protein level) or relatively (e.g., by comparing to the cancer associated polypeptide level in a second biological sample). Preferably, LRRC15 polypeptide expression level in the first biological sample is measured or estimated and compared to a standard LRRC15 polypeptide level, the standard being taken from a second biological sample obtained from an individual not having the disorder or being determined by averaging levels from a population of individuals not having the disorder. As will be appreciated in the art, once the “standard” LRRC15 polypeptide level is known, it can be used repeatedly as a standard for comparison.

By “biological sample” is intended any biological sample obtained from an individual, cell line, tissue culture, or other source of cells potentially expressing LRRC15. As indicated, biological samples include body fluids (such as sera, plasma, urine, synovial fluid and spinal fluid), and other tissue sources which contain cells potentially expressing LRRC15. Methods for obtaining tissue biopsies and body fluids from mammals are well known in the art.

In an additional embodiment, antibodies, or immunospecific fragments of antibodies directed to a conformational epitope of LRRC15 may be used to quantitatively or qualitatively detect the presence of LRRC15 gene products or conserved variants or peptide fragments thereof. This can be accomplished, for example, by immunofluorescence techniques employing a fluorescently labeled antibody coupled with light microscopic, flow cytometric, or fluorimetric detection.

Binding molecules for use in the diagnostic methods described above include any binding molecule which specifically binds to an LRRC15 gene product. Such gene products include, but are not limited to, an LRRC15 polypeptide comprising, consisting essentially of, or consisting of amino acids 1 to 581 of SEQ ID NO:2 or a fragment thereof, an LRRC15 variant polypeptide at least 70%, 75%, 80%, 85%, 90%, or 95% identical to amino acids 1 to 581 or SEQ ID NO:2 or a fragment thereof, an LRRC15 variant polypeptide at least 70%, 75%, 80%, 85%, 90%, or 95% identical to an LRRC15 polypeptide fragment comprising, consisting essentially of, or consisting of amino acids 22 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 22 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 22 to 538 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 1 to 538 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 581 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 537 of SEQ ID NO:2, an LRRC15 variant polypeptide at least 70%, 75%, 80%, 85%, 90%, or 95% identical to an LRRC15 polypeptide fragment comprising, consisting essentially of, or consisting of amino acids 2 to 538 of SEQ ID NO:2, and an LRRC15 messenger RNA comprising, consisting essentially of, or consisting of nucleotides 1 to 5938 of SEQ ID NO:5 or a fragment thereof.

In the above embodiments, exemplary “fragments” of an LRRC15 polypeptide consisting of amino acids 1 to 581 of SEQ ID NO:2 include, but are not limited to: a fragment comprising, consisting essentially of, or consisting of of amino acids 1 to 538 of SEQ ID NO:2, a fragment comprising, consisting essentially of, or consisting of amino acids 1 to 537 of SEQ ID NO:2, a fragment comprising, consisting essentially of, or consisting of amino acids 2 to 581 of SEQ ID NO:2, a fragment comprising, consisting essentially of, or consisting of amino acids 2 to 538 of SEQ ID NO:2, a fragment comprising, consisting essentially of, or consisting of amino acids 2 to 537 of SEQ ID NO:2, a fragment comprising, consisting essentially of, or consisting of amino acids 22 to 581 of SEQ ID NO:2, a fragment comprising, consisting essentially of, or consisting of amino acids 22 to 538 of SEQ ID NO:2, a fragment comprising, consisting essentially of, or consisting of amino acids 22 to 537 of SEQ ID NO:2, a fragment comprising, consisting essentially of, or consisting of amino acids 127 to 157 of SEQ ID NO:2, and a fragment comprising, consisting essentially of, or consisting of amino acids 480 to 509 of SEQ ID NO:2. Corresponding fragments of a variant LRRC15 polypeptide at least 70%, 75%, 80%, 85%, 90%, or 95% identical to amino acids 1 to 581 of SEQ ID NO:2 are also contemplated.

In the above embodiments, exemplary “fragments” of an LRRC15 polypeptide consisting of amino acids 1 to 581 of SEQ ID NO:2 include, but are not limited to: a fragment comprising, consisting essentially of, or consisting of from about amino acid 25 of SEQ ID NO:2 to about amino acid 60 of SEQ ID NO:2; from about amino acid 85 of SEQ ID NO:2 to about amino acid 95 of SEQ ID NO:2; from about amino acid 140 of SEQ ID NO:2 to about amino acid 170 of SEQ ID NO:2; from about amino acid 200 of SEQ ID NO:2 to about amino acid 215 of SEQ ID NO:2; from about amino acid 240 of SEQ ID NO:2 to about amino acid 255 of SEQ ID NO:2; from about amino acid 365 of SEQ ID NO:2 to about amino acid 380 of SEQ ID NO:2; from about amino acid 420 of SEQ ID NO:2 to about amino acid 430 of SEQ ID NO:2; and from about amino acid 480 of SEQ ID NO:2 to about amino acid 505 of SEQ ID NO:2; from about amino acid 25 of SEQ ID NO:2 to about amino acid 50 of SEQ ID NO:2; from about amino acid 75 of SEQ ID NO:2 to about amino acid 115 of SEQ ID NO:2; from about amino acid of SEQ ID NO:2145 to about amino acid 175 of SEQ ID NO:2; from about amino acid 195 of SEQ ID NO:2 to about amino acid 215 of SEQ ID NO:2; from about amino acid 225 of SEQ ID NO:2 to about amino acid 270 of SEQ ID NO:2; from about amino acid 285 of SEQ ID NO:2 to about amino acid 325 of SEQ ID NO:2; from about amino acid 326 of SEQ ID NO:2 to about amino acid 385 of SEQ ID NO:2; from about amino acid 386 of SEQ ID NO:2 to about amino acid 450 of SEQ ID NO:2; and from about amino acid 475 of SEQ ID NO:2 to about amino acid 540 of SEQ ID NO:2.

Additional exemplary “fragments” of an LRRC15 peptide consisting of amino acids 1 to 581 of SEQ ID NO:2 include, but are not limited to those fragments comprising, consisting essentially of, or consisting of one or more leucine-rich-repeat (LRR) regions of LRRC15. Such fragments, include, for example, a fragment comprising, consisting essentially of, or consisting of amino acids 51 to 75 of SEQ ID NO:2 (LRR1); a fragment comprising, consisting essentially of, or consisting of amino acids 76 to 99 of SEQ ID NO:2 (LRR2); a fragment comprising, consisting essentially of, or consisting of amino acids 100 to 123 of SEQ ID NO:2 (LRR3); a fragment comprising, consisting essentially of, or consisting of amino acids 125 to 147 of SEQ ID NO:2 (LRR4); a fragment comprising, consisting essentially of, or consisting of amino acids 148 to 171 of SEQ ID NO:2 (LRR5); a fragment comprising, consisting essentially of, or consisting of amino acids 173 to 195 of SEQ ID NO:2 (LRR6); a fragment comprising, consisting essentially of, or consisting of amino acids 196 to 219 of SEQ ID NO:2 (LRR7); a fragment comprising, consisting essentially of, or consisting of amino acids 221 to 243 of SEQ ID NO:2 (LRR8); a fragment comprising, consisting essentially of, or consisting of amino acids 244 to 267 of SEQ ID NO:2 (LRR9); a fragment comprising, consisting essentially of, or consisting of amino acids 269 to 291 of SEQ ID NO:2 (LRR10); a fragment comprising, consisting essentially of, or consisting of amino acids 292 to 315 of SEQ ID NO:2 (LRR11); a fragment comprising, consisting essentially of, or consisting of amino acids 317 to 339 of SEQ ID NO:2 (LRR12); a fragment comprising, consisting essentially of, or consisting of amino acids 340 to 363 of SEQ ID NO:2 (LRR13); a fragment comprising, consisting essentially of, or consisting of amino acids 364 to 387 of SEQ ID NO:2 (LRR14); and a fragment comprising, consisting essentially of, or consisting of amino acids 389 to 411 of SEQ ID NO:2 (LRR15). Corresponding fragments of a variant LRRC15 polypeptide at least 70%, 75%, 80%, 85%, 90%, or 95% identical to amino acids 1 to 581 of SEQ ID NO:2 are also contemplated.

Additional exemplary “fragments” of an LRRC15 peptide consisting of amino acids 1 to 581 of SEQ ID NO:2 include, but are not limited to those fragments comprising, consisting essentially of, or consisting of amino acids x to y, where x is any of amino acids 12, 13, 14, 15, 16, 17, 18, 19, 20, 21, 22, 23, 24, 25, 26, 27, 28, 29, 30, 31, or 32 of SEQ ID NO:2, and where y is any of amino acids 528, 539, 530, 531, 532, 533, 534, 535, 536, 537, 538, 539, 540, 541, 542, 543, 544, 545, 546, 547, or 548 of SEQ ID NO:2. Corresponding fragments of a variant LRRC15 polypeptide at least 70%, 75%, 80%, 85%, 90%, or 95% identical to amino acids 1 to 581 of SEQ ID NO:2 are also contemplated.

Exemplary “fragments” of an LRRC15 messenger RNA comprising, consisting essentially of, or consisting of nucleotides 1 to 5938 of SEQ ID NO:5 include, but are not limited to those fragments comprising, consisting essentially of, or consisting of nucleotides 1 to 143 of SEQ ID NO:5, nucleotides 144 to 1889 of SEQ ID NO:5 and nucleotides 1890 to 5938 of SEQ ID NO:5. Additional fragments include, but are not limited to fragments which comprise, consist essentially of, or consist of at least 5, and least 10, at least 25, at least 50, at least 100, at least 250 at least 500, at least 750 at least 1000, at least 2000, at least 3000, at least 4000, or at least 5000 nucleotides of SEQ ID NO:5. Such nucleotides may be contiguous, i.e., in a linear sequence, or non-contiguous, i.e., from at least two different regions of a nucleic acid brought together by, e.g., secondary structure.

By “a binding molecule which specifically binds to an LRRC messenger RNA comprising, consisting essentially of, or consisting of nucleotides 1 to 5938 of SEQ ID NO:5 or a fragment thereof” is meant any molecule that binds to such a nucleic acid, and includes nucleic acid binding molecules, polypeptide binding molecules, and small molecule binding molecules. Nucleic acid binding molecules typically bind to a LRRC15 mRNA or fragment thereof as described above by hybridization. Such nucleic acid binding molecules include, but are not limited to antisense nucleic acids, siRNA constructs, and ribozymes, e.g., hammerhead ribozymes. Such binding molecules typically hybridize to their complementary nucleic acids under standard hybridiztion conditions as described herein.

Other binding molecules for use in the diagnostic methods described herein include binding molecules which specifically bind to at least one epitope of LRRC15, where the epitope comprises, consists essentially of, or consists of at least about four to five amino acids amino acids of SEQ ID NO:2, at least seven, at least nine, or between at least about 15 to about 30 amino acids of SEQ ID NO:2. The amino acids of a given epitope of SEQ ID NO:2 as described may be, but need not be contiguous. In certain embodiments, the at least one epitope of LRRC15 comprises, consists essentially of, or consists of a non-linear epitope formed by the extracellular domain of LRRC15 as expressed on the surface of a cell. Thus, in certain embodiments the at least one epitope of LRRC15 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

Additional binding molecules include those which specifically bind to at least one epitope of LRRC15, where the epitope comprises, consists essentially of, or consists of, in addition to one, two, three, four, five, six or more contiguous or non-contiguous amino acids of SEQ ID NO:2 as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

Cancers that may be diagnosed, and/or prognosed using the methods described above include but are not limited to, colorectal cancer, breast cancer, ovarian cancer, prostate cancer, pancreatic cancer, lung cancer, liver cancer, uterine cancer, and/or skin cancer.

VI. Antibodies or Immunospecific Fragments Thereof

In one embodiment, a binding molecule for use in the methods of the invention is an antibody molecule, or immunospecific fragment thereof. Unless it is specifically noted, as used herein a “fragment thereof” in reference to an antibody refers to an immunospecific fragment, i.e., an antigen-specific fragment. In one embodiment, a binding molecule, e.g., an antibody of the invention is a bispecific binding molecule, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific binding molecule, binding polypeptide, or antibody has at least one binding domain specific for at least one epitope on a target polypeptide disclosed herein, e.g., LRRC15. In another embodiment, a bispecific binding molecule, binding polypeptide, or antibody has at least one binding domain specific for an epitope on a target polypeptide and at least one target binding domain specific for a drug or toxin. In yet another embodiment, a bispecific binding molecule, binding polypeptide, or antibody has at least one binding domain specific for an epitope on a target polypeptide disclosed herein, and at least one binding domain specific for a prodrug. A bispecific binding molecule, binding polypeptide, or antibody may be a tetravalent antibody that has two target binding domains specific for an epitope of a target polypeptide disclosed herein and two target binding domains specific for a second target. Thus, a tetravalent bispecific binding molecule, binding polypeptide, or antibody may be bivalent for each specificity.

Antibody binding molecules for use in the treatment methods of the present invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, methods of treating hyperproliferative diseases according to the present invention comprise administration of an antibody, or immunospecific fragment thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the C_{H}2 domain will be deleted.

In certain antibodies or immunospecific fragments thereof for use in the diagnostic and therapeutic methods described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of antibodies or immunospecific fragments thereof for use in the diagnostic and therapeutic methods disclosed herein can be made from whole precursor or parent antibodies using techniques known in the art. Exemplary techniques are discussed in more detail herein.

In certain embodiments both the variable and constant regions of LRRC15-specific antibodies or immunospecific fragments thereof for use in the diagnostic and treatment methods disclosed herein are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human anti bodies can likewise be produced by various display technologies, e.g., phage display or other viral display systems, as described in more detail elsewhere herein.

Binding molecules, e.g., binding polypeptides, e.g., LRRC15-specific antibodies or immunospecific fragments thereof for use in the diagnostic and treatment methods disclosed herein can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Binding molecules, e.g., binding polypeptides, e.g., LRRC15-specific antibodies or immunospecific fragments thereof for use in the diagnostic and treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In preferred embodiments, a binding molecule, e.g., a binding polypeptide, e.g., a LRRC15-specific antibody or immunospecific fragment thereof for use in the diagnostic and treatment methods disclosed herein will not elicit a deleterious immune response in the animal to be treated, e.g., in a human. In one embodiment, binding molecules, e.g., binding polypeptides, e.g., LRRC15-specific antibodies or immunospecific fragments thereof for use in the diagnostic and treatment methods disclosed herein be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., LRRC15-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In the therapeutic methods described herein,, administration is to an animal, e.g., a human, in need of treatment for cancer or other hyperproliferative disorder. For example, a binding molecule, e.g., a binding polypeptide, e.g., a LRRC15-specific antibody or immunospecific fragment thereof may be administered to a human patient diagnosed with a tumor, other cancerous lesion, or other hyperproliferative disorder, a human patient who has been treated for cancer and is in remission, but is in need of further chronic treatment to prevent recurrence or spread of cancer, a human who exhibits early warning signs for a certain cancer or hyperproliferative disorder and is a candidate for preventative treatment, or preventatively to a human who is genetically predisposed to contract a certain cancer.

The methods of treatment of hyperproliferative disorders as described herein are typically tested in vitro, and then in vivo in an acceptable animal model, for the desired therapeutic or prophylactic activity, prior to use in humans. Suitable animal models, including transgenic animals, are will known to those of ordinary skill in the art. For example, in vitro assays to demonstrate the therapeutic utility of binding molecule described herein include the effect of a binding molecule on a cell line or a patient tissue sample. The effect of the binding molecule on the cell line and/or tissue sample can be determined utilizing techniques known to those of skill in the art including, but not limited to, apoptosis assays and cell lysis assays. In accordance with the invention, in vitro assays which can be used to determine whether administration of a specific binding molecule is indicated, include in vitro cell culture assays in which a patient tissue sample is grown in culture, and exposed to or otherwise administered a compound, and the effect of such compound upon the tissue sample is observed.

Antibodies or fragments thereof for use as therapeutic binding molecules may be generated by any suitable method known in the art. Polyclonal antibodies to an antigen of interest can be produced by various procedures well known in the art. For example, a binding molecule, e.g., a binding polypeptide, e.g., a LRRC15-specific antibody or immunospecific fragment thereof can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified tumor associated antigens such as LRRC15 or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a desired target polypeptide, e.g., LRRC15.

Antibody fragments that recognize specific epitopes may be generated by known techniques. For example, Fab and F(ab′)2 fragments may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the C_{H}1 domain of the heavy chain.

Those skilled in the art will also appreciate that DNA encoding antibodies or antibody fragments (e.g., antigen binding sites) may also be derived from antibody phage libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. In particular, DNA sequences encoding V_{H} and V_{L} regions are amplified from animal cDNA libraries (e.g., human or murine cDNA libraries of lymphoid tissues) or synthetic cDNA libraries. In certain embodiments, the DNA encoding the V_{H} and V_{L} regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} or V_{L} regions are usually recombinantly fused to either the phage gene II or gene VIII. Phage expressing an antigen binding domain that binds to an antigen of interest (i.e., a LRRC15 polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332.

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., LRRC15. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the V_{H} and V_{L} genes can be amplified using, e.g., RT-PCR. The V_{H} and V_{L} genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the diagnostic and therapeutic methods disclosed herein can be produced by any method known in the art for the synthesis of antibodies, in particular, by chemical synthesis or preferably, by recombinant expression techniques as described herein.

It will further be appreciated that the scope of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Recombinant expression of an antibody, or fragment, derivative or analog thereof, e.g., a heavy or light chain of an antibody which binds to a target molecule described herein, e.g., LRRC15, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993);, TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Prolocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in U.S. 2002 0123057 A1.

In one embodiment, a binding molecule or antigen binding molecule for use in the methods of the invention comprises a synthetic constant region wherein one or more domains are partially or entirely deleted (“domain-deleted antibodies”). In certain embodiments compatible modified antibodies will comprise domain deleted constructs or variants wherein the entire C_{H}2 domain has been removed (ΔC_{H}2 constructs). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody.

In certain embodiments, modified antibodies for use in the methods disclosed herein are minibodies. Minibodies can be made using methods described in the art (see, e.g., see e.g., U.S. Pat. No. 5,837,821 or WO 94/09817A1).

In another embodiment, modified antibodies for use in the methods disclosed herein are C_{H}2 domain deleted antibodies which are known in the art. Domain deleted constructs can be derived using a vector (e.g., from Biogen IDEC Incorporated) encoding an IgG₁ human constant domain (see, e.g., WO 02/060955A2 and WO02/096948A2). This exemplary vector was engineered to delete the C_{H}2 domain and provide a synthetic vector expressing a domain deleted IgG₁ constant region.

In one embodiment, a binding molecule, e.g., a binding polypeptide, e.g., a LRRC15-specific antibody or immunospecific fragment thereof for use in the diagnostic and treatment methods disclosed herein comprises an immunoglobulin heavy chain having deletion or substitution of a few or even a single amino acid as long as it permits association between the monomeric subunits. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides the use of antibodies that comprise, consist essentially of, or consist of, variants (including derivatives) of antibody molecules (e.g., the V_{H} regions and/or V_{L} regions) described herein, which antibodies or fragments thereof immunospecifically bind to a LRRC15 polypeptide or fragment or variant thereof. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid subsitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR1, V_{H}CDR2, V_{H}CDR3, V_{L} region, V_{L}CDR1, V_{L}CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a LRRC15 polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a LRRC15 polypeptide) can be determined using techniques described herein or by routinely modifying techniques known in the art.

VII. Fusion Proteins and Antibody Conjugates

As discussed in more detail elsewhere herein, binding molecules, e.g., binding polypeptides, e.g., LRRC15-specific antibodies or immunospecific fragments thereof for use in the diagnostic and treatment methods disclosed herein may further be recombinantly fused to a heterologous polypeptide at the N- or C-terminus or chemically conjugated (including covalent and non-covalent conjugations) to polypeptides or other compositions. For example, LRRC15-specific binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Binding molecules, e.g., binding polypeptides, e.g., LRRC15-specific antibodies or immunospecific fragments thereof for use in the diagnostic and treatment methods disclosed herein include derivatives that are modified, i.e., by the covalent attachment of any type of molecule to the antibody such that covalent attachment does not prevent the antibody binding LRRC15. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Binding molecules, e.g., binding polypeptides, e.g., LRRC15-specific antibodies or immunospecific fragments thereof for use in the diagnostic and treatment methods disclosed herein can be composed of amino acids joined to each other by peptide bonds or modified peptide bonds, i.e., peptide isosteres, and may contain amino acids other than the 20 gene-encoded amino acids. LRRC15-specfic antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the LRRC15-specific antibody, including the peptide backbone, the amino acid side-chains and the amino or carboxyl termini, or on moieties such as carbohydrates. It will be appreciated that the same type of modification may be present in the same or varying degrees at several sites in a given LRRC15-specific antibody. Also, a given LRRC15-specific antibody may contain many types of modifications. LRRC15-specific antibodies may be branched, for example, as a result of ubiquitination, and they may be cyclic, with or without branching. Cyclic, branched, and branched cyclic LRRC15-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising, consisting essentially of, or consisting of, an antibody (including molecules comprising, consisting essentially of, or consisting of, antibody fragments or variants thereof), that immunospecifically binds to LRRC15, and a heterologous polypeptide. Preferably, the heterologous polypeptide to which the antibody is fused is useful for function or is useful to target the LRRC15 polypeptide expressing cells, including but not limited to breast, ovarian, bladder, colon, lung, prostate, and pancreatic cancer cell. In an alternative preferred embodiment, the heterologous polypeptide to which the antibody is fused is useful for T cell, macrophage, and/or monocyte cell function or is useful to target the antibody to a T cell, macrophage, or monocyte. In one embodiment, a fusion protein of the invention comprises, consists essentially of, or consists of, a polypeptide having the amino acid sequence of any one or more of the V_{H} regions of an antibody of the invention or the amino acid sequence of any one or more of the V_{L} regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three, or more of the V_{H} CDRs of an LRRC15-specific antibody, or the amino acid sequence of any one, two, three, or more of the V_{L} CDRs of an LRRC15-specific antibody, or fragments or variants thereof, and a heterologous polypeptide sequence. In one embodiment, the fusion protein comprises, consists essentially of, or consists of a polypeptide having the amino acid sequence of a V_{H} CDR3 of an LRRC15-specific antibody, or fragment or variant thereof, and a heterologous polypeptide sequence, which fusion protein specifically binds to at least one epitope of LRRC15. In another embodiment, a fusion protein comprises, consists essentially of, or consists of a polypeptide having the amino acid sequence of at least one V_{H} region of an LRRC15-specific antibody and the amino acid sequence of at least one V_{L} region of an LRRC15-specific antibody or fragments or variants thereof, and a heterologous polypeptide sequence. Preferably, the V_{H} and V_{L} regions of the fusion protein correspond to a single source antibody (or scFv or Fab fragment) which specifically binds at least one epitope of LRRC15. In yet another embodiment, a fusion protein for use in the diagnostic and treatment methods disclosed herein comprises, consists essentially of, or consists of a polypeptide having the amino acid sequence of any one, two, three or more of the V_{H} CDRs of an LRRC15-specific antibody and the amino acid sequence of any one, two, three or more of the V_{L} CDRs of an LRRC15-specific antibody, or fragments or variants thereof, and a heterologous polypeptide sequence. Preferably, two, three, four, five, six, or more of the V_{H} CDR(s) or V_{L} CDR(s) correspond to single source antibody (or scFv or Fab fragment) of the invention. Nucleic acid molecules encoding these fusion proteins are also encompassed by the invention.

The invention also pertains to the use of binding molecules which comprise one or more immunoglobulin domains. Fusion proteins for use in the diagnostic and therapeutic methods disclosed herein comprise a binding domain (which comprises at least one binding site) and a dimerization domain (which comprises at least one heavy chain portion). The subject fusion proteins may be bispecific (with one binding site for a first target and a second binding site for a second target) or may be multivalent (with two binding sites for the same target).

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

In one embodiment a fusion protein combines the binding domain(s) of the ligand or receptor (e.g. the extracellular domain (ECD) of a receptor) with at least one heavy chain domain and a synthetic connecting peptide. In one embodiment, when preparing the fusion proteins of the present invention, nucleic acid encoding the binding domain of the ligand or receptor domain will be fused C-terminally to nucleic acid encoding the N-terminus of an immunoglobulin constant domain sequence. N-terminal fusions are also possible. In one embodiment, a fusion protein includes a C_{H}2 and a C_{H}3 domains. Fusions may also be made to the C-terminus of the Fc portion of a constant domain, or immediately N-terminal to the C_{H}1 of the heavy chain or the corresponding region of the light chain.

In one embodiment, the sequence of the ligand or receptor binding domain is fused to the N-terminus of the Fc domain of an immunoglobulin molecule. It is also possible to fuse the entire heavy chain constant region to the ligand or receptor binding domain sequence. In one embodiment, a sequence beginning in the hinge region just upstream of the papain cleavage site which defines IgG Fc chemically (i.e. residue 216, taking the first residue of heavy chain constant region to be 114 according to the Kabat system), or analogous sites of other immunoglobulins is used in the fusion. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins can be assembled as multimers, and particularly as heterodimers or heterotetramers. Generally, these assembled immunoglobulin-like proteins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

As discussed elsewhere herein, binding molecules, e.g., binding polypeptides, e.g., LRRC15-specific antibodies or immunospecific fragments thereof for use in the diagnostic and treatment methods disclosed herein may be fused to heterologous polypeptides to increase the in vivo half life of the polypeptides or for use in immunoassays using methods known in the art. In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5 receptor, have been fused with Fc portions for the purpose of high-throughput screening assays to identify antagonists of hIL-5. (See, D. Bennett et al., J. Molecular Recognition 8:52-58 (1995); K. Johanson et al. J. Biol. Chem. 270:9459-9471 (1995).

Moreover, binding molecules, e.g., binding polypeptides, e.g., LRRC15-specific antibodies or immunospecific fragments thereof for use in the diagnostic and treatment methods disclosed herein can be fused to marker sequences, such as a peptide to facilitates their purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). Ordinarily, the ligand or ligand binding partner is fused C-terminally to the N-terminus of the constant region of the heavy chain (or heavy chain portion) and in place of the variable region. Any transmembrane regions or lipid or phospholipids anchor recognition sequences of ligand binding receptor are preferably inactivated or deleted prior to fusion. DNA encoding the ligand or ligand binding partner is cleaved by a restriction enzyme at or proximal to the 5′ and 3′ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

Binding molecules for use in the methods of the present invention may be used in non-conjugated form or may be conjugated to at least one of a variety of molecules, e.g., to improve the therapeutic properties of the molecule, to facilitate target detection, or for imaging or therapy of the patient. Binding molecules, e.g., binding polypeptides, e.g., LRRC15-specific antibodies or immunospecific fragments thereof for use in the diagnostic and treatment methods disclosed herein can be labeled or conjugated either before or after purification, when purification is performed.

In particular, binding molecules, e.g., binding polypeptides, e.g., LRRC15-specific antibodies or immunospecific fragments thereof for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule, e.g., a binding polypeptide, e.g., a LRRC15-specific antibody or immunospecific fragment thereof for use in the diagnostic and treatment methods disclosed herein can be conjugated to a molecule that decreases vascularization of tumors. In other embodiments, the disclosed compositions may comprise binding molecules, e.g., binding polypeptides, e.g., LRRC15-specific antibodies or immunospecific fragments thereof coupled to drugs or prodrugs. Still other embodiments of the present invention comprise the use of binding molecules, e.g., binding polypeptides, e.g., LRRC15-specific antibodies or immunospecific fragments thereof conjugated to specific biotoxins or their cytotoxic fragments such as ricin, gelonin, pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukernias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ⁸⁶Re and ⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules, e.g., binding polypeptides, e.g., LRRC15-specific antibodies or immunospecific fragments thereof may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Binding molecules, e.g., binding polypeptides, e.g., LRRC15-specific antibodies or immunospecific fragments thereof for use in the diagnostic and treatment methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylene-triaminepentaacetic acid), which comprises a 1:1 mixture of 1-isothiocyanato-benzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of 131I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Those skilled in the art will appreciate that non-radioactive conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the binding molecules, e.g., binding polypeptides, e.g., LRRC15-specific antibodies or immunospecific fragments thereof with cytostatic/cytotoxic substances and metal chelates are prepared in an analogous manner.

Additional preferred agents for conjugation to binding molecules, e.g., binding polypeptides, e.g., LRRC15-specific antibodies or immunospecific fragments thereof are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that binding molecules, e.g., binding polypeptides, e.g., LRRC15-specific antibodies or immunospecific fragments thereof for use in the diagnostic and treatment methods disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules, e.g., binding polypeptides, e.g., LRRC15-specific antibodies or immunospecific fragments thereof disclosed herein comprise cytokines such as lymphokines and interferons. In view of the instant disclosure it is submitted that one skilled in the art could readily form such constructs using conventional techniques.

Another class of compatible cytotoxins that may be used in in association with or conjugated to the disclosed binding molecules, e.g., binding polypeptides, e.g., LRRC15-specific antibodies or immunospecific fragments thereof, are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., a LRRC15-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules, e.g., binding polypeptides, e.g., LRRC15-specific antibodies or immunospecific fragments conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, e.g., binding polypeptide, e.g., LRRC15-specific antibody or immunospecific fragment thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule, e.g., a binding polypeptide, e.g., a LRRC15-specific antibody or immunospecific fragment thereof also can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a binding molecule, e.g., a binding polypeptide, e.g., a LRRC15-specific antibody or immunospecific fragment thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, e.g., binding polypeptide, e.g., LRRC15-specific antibody or immunospecific fragment thereof, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule, e.g., a binding polypeptide, e.g., a LRRC15-specific antibody or immunospecific fragment thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a binding molecule, e.g., a binding polypeptide, e.g., a LRRC15-specific antibody or immunospecific fragment thereof are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

VIII. Polynucleotides Encoding LRRC15—Specific Binding Molecules

The present invention also provides for nucleic acid molecules encoding LRRC15-specific antibodies or other binding molecules (including molecules comprising, consisting essentially of, or consisting of, antibody fragments or variants thereof).

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or other binding molecule may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody or other binding molecule, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other binding molecule. Amplified nucleic acids generated by PCR may then be cloned into replicable cloning vectors using any method well known in the art.

Once the nucleotide sequence and corresponding amino acid sequence of the antibody or other binding molecule is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an LRRC15-specific antibody or other binding molecule can be composed of any polyribonucleotide or polydeoxribonucleotide, which may be unmodified RNA or DNA or modified RNA or DNA. For example, a polynucleotide encoding an LRRC15-specific antibody or other binding molecule can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an LRRC15-specific antibody can be composed of triple-stranded regions comprising RNA or DNA or both RNA and DNA. A polynucleotide encoding an LRRC15-specific antibody or other binding molecule may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

IX. Antibody Expression

Following manipulation of the isolated genetic material to provide binding molecules, e.g., binding polypeptides, e.g., LRRC15-specific antibodies or immunospecific fragments thereof for use in the diagnostic and treatment methods disclosed herein, the polynucleotides encoding the binding molecules are typically inserted in an expression vector for introduction into host cells that may be used to produce the desired quantity of binding molecule.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF/1His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the binding molecules, e.g., binding polypeptides, e.g., LRRC15-specific antibodies or immunospecific fragments thereof for use in the diagnostic and treatment methods disclosed herein may be expressed using polycistronic constructs such as those disclosed in U.S. Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of binding molecules, e.g., binding polypeptides, e.g., LRRC15-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of binding molecules disclosed in the instant application.

More generally, once the vector or DNA sequence encoding a monomeric subunit of the binding polypeptide (e.g. a modified antibody) has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

Along those same lines, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding binding molecules, e.g., binding polypeptides, e.g., LRRC15-specific antibodies or immunospecific fragments thereof for use in the diagnostic and treatment methods disclosed herein can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologouspolypeptides must be isolated, purified and then assembled into functional molecules. Where tetravalent forms of antibodies are desired, the subunits will then self-assemble into tetravalent antibodies (WO02/096948A2).

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

X. Immunoassays

Binding molecules, e.g., binding polypeptides, e.g., LRRC15-specific antibodies or immunospecific fragments thereof for use in the diagnostic and treatment methods disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4.degree. C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²¹I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

LRRC15-specific binding molecules may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled LRRC15-specific antibody or fragment thereof, preferably applied by overlaying the labeled antibody (or fragment) onto a biological sample. Through the use of such a procedure, it is possible to determine not only the presence of LRRC15 protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for LRRC15 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to LRRC15 or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled LRRC15-specific antibody. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of LRRC15-specific antibody may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

XI. Pharmaceutical Compositions and Administration Methods

Methods of preparing and administering binding molecules, e.g., binding polypeptides, e.g., LRRC15-specific antibodies or immunospecific fragments thereof to a subject in need thereof are well known to or are readily determined by those skilled in the art. The route of administration of the binding molecule, e.g., binding polypeptide, e.g., LRRC15-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., LRRC15-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., a LRRC15-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (U.S.-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or other binding molecule, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Binding molecules, e.g., binding polypeptides, e.g., LRRC15-specific antibodies or immunospecific fragments thereof for use in the diagnostic and treatment methods disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moeity, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., LRRC15-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a binding molecule, e.g., a binding polypeptide, e.g., a LRRC15-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Specific embodiments comprise a method of treating a hyperproliferative disorder, comprising administering to an animal in need of treatment an effective amount of a binding molecule which specifically binds to a polynucleotide which encodes LRRC15, or a fragment or a variant thereof. Such binding molecules include, but are not limited to antisense molecules, ribozymes, siRNA, and RNAi. Typically, such binding molecules are separately administered to the animal (see, for example, O'Connor, J. Neurochem. 56:560 (1991), but such binding molecules may also be expressed in vivo from polynucleotides taken up by a host cell and expressed in vivo. See also Oligodeoxynucleotides as Antisense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J. Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes a mature target polypeptide disclosed herein, e.g., LRRC15, may be used to design an antisense RNA oligonucleotide of from about 10 to 40 base pairs in length. A DNA oligonucleotide is designed to be complementary to a region of the gene involved in transcription thereby preventing transcription and the production of the target protein. The antisense RNA oligonucleotide hybridizes to the mRNA in vivo and blocks translation of the mRNA molecule into the target polypeptide.

In one embodiment, antisense nucleic acids specific for an LRRC15-specific gene product are produced intracellularly by transcription from an exogenous sequence. For example, a vector or a portion thereof, is transcribed, producing an antisense nucleic acid (RNA). Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the antisense molecule, can be by any promoter known in the art to act in vertebrate, preferably human cells, such as those described elsewhere herein.

Absolute complementarity of an antisense molecule, although preferred, is not required. A sequence “complementary to at least a portion of an RNA encoding a target polypeptide,” referred to herein, means a sequence having sufficient complementarity to be able to hybridize with the RNA, forming a stable duplex; or triplex formation may be assayed. The ability to hybridize will depend on both the degree of complementarity and the length of the antisense nucleic acid. Generally, the larger the hybridizing nucleic acid, the more base mismatches it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of a messenger RNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature 372:333-335 (1994). Thus, oligonucleotides complementary to either the 5′- or 3′-non-translated, non-coding regions could be used in an antisense approach to inhibit translation of a target polypeptide disclosed herein, e.g., LRRC15. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Polynucleotides for use the therapeutic and diagnostic methods disclosed herein can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. 84:648-652 (1987)); PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., BioTechniques 6:958-976 (1988)) or intercalating agents. (See, e.g., Zon, Pharm. Res. 5:539-549(1988)). To this end, the oligonucleotide may be conjugated to another molecule, e.g., a peptide, hybridization triggered cross-linking agent, transport agent, hybridization-triggered cleavage agent, etc.

An antisense oligonucleotide for use in the therapeutic and diagnostic methods disclosed herein may comprise at least one modified base moiety which is selected from the group including, but not limited to, 5fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5′methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3(3-amino-3-N-2-carboxypropyl) uracil, (acp3)w, and 2,6-diaminopurine.

An antisense oligonucleotide for use in the therapeutic and diagnostic methods disclosed herein may also comprise at least one modified sugar moiety selected from the group including, but not limited to, arabinose, 2-fluoroarabinose, xylulose, and hexose.

In yet another embodiment, an antisense oligonucleotide for use in the therapeutic and diagnostic methods disclosed herein comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, an antisense oligonucleotide for use in the therapeutic and diagnostic methods disclosed herein is an a-anomeric oligonucleotide. An a-anomeric oligonucleotide forms specific double-stranded hybrids with complementary RNA in which, contrary to the usual situation, the strands run parallel to each other (Gautier et al., Nucl. Acids Res. 15:6625-6641(1987)). The oligonucleotide is a 2′-O-methylribonucleotide (Inoue et al., Nucl. Acids Res. 15:6131-6148(1987)), or a chimeric RNA-DNA analogue (Inoue et al., FEBS Lett. 215:327-330(1987)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451(1988)), etc.

Polynucleotide compositions for use in the diagnostic and therapeutic methods disclosed herein further include catalytic RNA, or a ribozyme (See, e.g., PCT International Publication WO 90/11364, published Oct. 4, 1990; Sarver et al., Science 247:1222-1225 (1990). The use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the target mRNA; i.e., to increase efficiency and minimize the intracellular accumulation of non-functional mRNA transcripts.

As in the antisense approach, ribozymes for use in the diagnostic and therapeutic methods disclosed herein can be composed of modified oligonucleotides (e.g. for improved stability, targeting, etc.) and may be delivered to cells which express LRRC15 in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous LRRC15 messages and inhibit translation. Since ribozymes unlike antisense molecules, are catalytic, a lower intracellular concentration is required for efficiency.

Polynucleotide compositions as described above may be employed to inhibit cell growth and proliferation of neoplastic cells and tissues expressing LRRC15. For example, such compositions may be used to inhibit the stimulation of angiogenesis of tumors, and, therefore, retard or prevent abnormal cellular growth and proliferation, for example, in tumor formation or growth.

Thus, the invention provides a method of treating hyperproliferative disorders or diseases in an animal, comprising administering to an animal in need of treatment (a) an antisense molecule directed to a polynucleotide encoding LRRC15, and/or (b) a ribozyme directed to a polynucleotide encoding LRRC15.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where LRRC15-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

Agents of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled binding polypeptides range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ²⁷⁹Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, 47Sc, Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ²⁵¹I, ²³¹I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not binding molecules, e.g., binding polypeptides, e.g., LRRC15-specific antibodies or immunospecific fragments thereof for use in the diagnostic and treatment methods disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, binding molecules, e.g., binding polypeptides, e.g., LRRC15-specific antibodies or immunospecific fragments thereof for use in the treatment methods disclosed herein are useful in a conjugated or unconjugated form in patients that have previously undergone adjunct therapies such as external beam radiation or chemotherapy. In other preferred embodiments, binding molecules, e.g., binding polypeptides, e.g., LRRC15-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While binding molecules, e.g., binding polypeptides, e.g., LRRC15-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of binding molecules, e.g., binding polypeptides, e.g., LRRC15-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of binding molecules, e.g., binding polypeptides, e.g., LRRC15-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and binding molecule, e.g., binding polypeptide, e.g., LRRC15-specific antibody or immunospecific fragment thereof, may be administered in any order or concurrently. In selected embodiments binding molecules, e.g., binding polypeptides, e.g., LRRC15-specific antibodies or immunospecific fragments thereof for use in treatment methods disclosed herein will be administered to patients that have previously undergone chemotherapy. In yet other embodiments, binding molecules, e.g., binding polypeptides, e.g., LRRC15-specific antibodies or immunospecific fragments thereof for use in treatment methods disclosed herein will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated binding molecules, e.g., binding polypeptides, e.g., LRRC15-specific antibodies or immunospecific fragments thereof for use in treatment methods disclosed herein may be used to effectively treat patients having ANCs lower than about 2000/mm³ or platelet counts lower than about 150,000/mm³. More preferably binding molecules, e.g., binding polypeptides, e.g., LRRC15-specific antibodies or immunospecific fragments thereof for use in treatment methods disclosed herein may be used to treat patients having ANCs of less than about 1500/mm³, less than about 1000/m³ or even more preferably less than about 500/mm³. Similarly, binding molecules, e.g., binding polypeptides, e.g., LRRC15-specific antibodies or immunospecific fragments thereof for use in treatment methods disclosed herein may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed binding molecules, e.g., binding polypeptides, e.g., LRRC15-specific antibodies or immunospecific fragments thereof for use in treatment methods disclosed herein may be used to treat disorders in patients exhibiting myelosuppression regardless of the cause.

In this regard it will further be appreciated that binding molecules, e.g., binding polypeptides, e.g., LRRC15-specific antibodies or immunospecific fragments thereof for use in treatment methods disclosed herein may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more binding molecules, e.g., binding polypeptides, e.g., LRRC15-specific antibodies or immunospecific fragments thereof as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the binding molecules disclosed herein may vary by subject or may be administered according to what is known in the art. See for example, Bruce A Chabner et al., Antineoplastic Agents, in Goodman & Gilman's The Pharmacological Basis of Therapeutics 1233-1287 ((Joel G. Hardman et al., eds., 9^{th} ed. (1996).

As previously discussed, binding molecules, e.g., binding polypeptides, e.g., LRRC15-specific antibodies or immunospecific fragments thereof, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of binding molecule, e.g., binding polypeptide, e.g., LRRC15-specific antibody or immunospecific fragment thereof, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, binding molecules, e.g., binding polypeptides, e.g., LRRC15-specific antibodies or immunospecific fragments thereof for use in treatment methods disclosed herein may be administered to a human or other animal in accordance with the aforementioned methods of treatment in an amount sufficient to produce a therapeutic or prophylactic effect. The binding molecules, e.g., binding polypeptides, e.g., LRRC15-specific antibodies or immunospecific fragments thereof for use in treatment methods disclosed herein can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

All of the references cited above, as well as all references cited herein, are incorporated herein by reference in their entireties.

### EXAMPLES

### Example 1

### GSIB4 Histochemistry

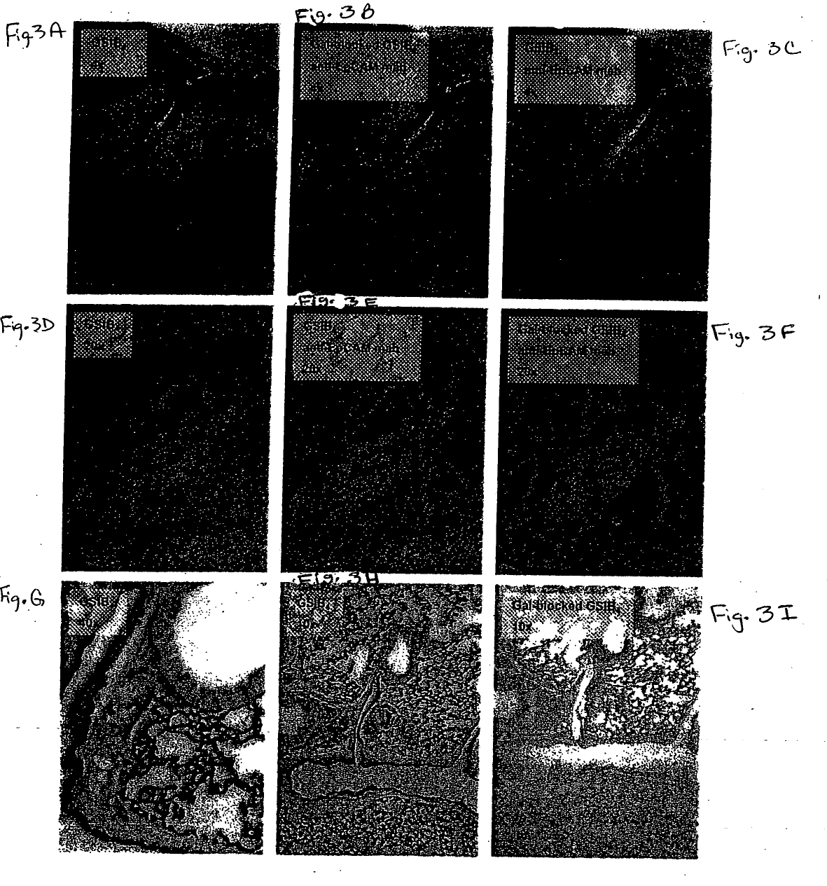

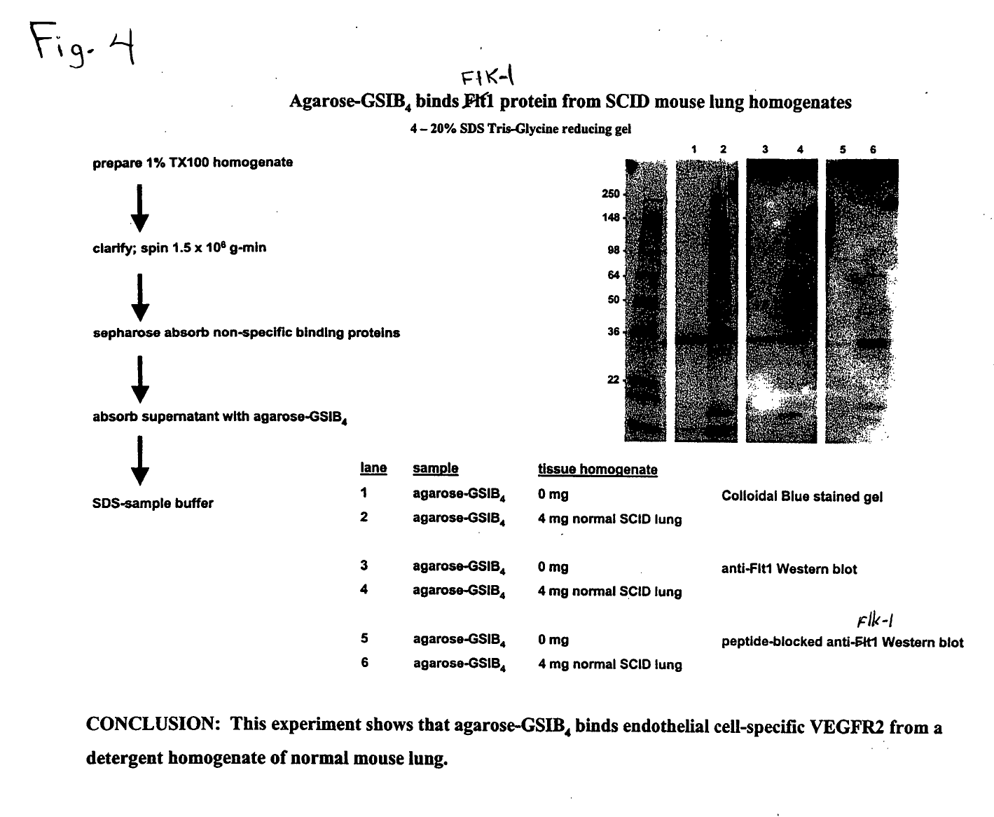

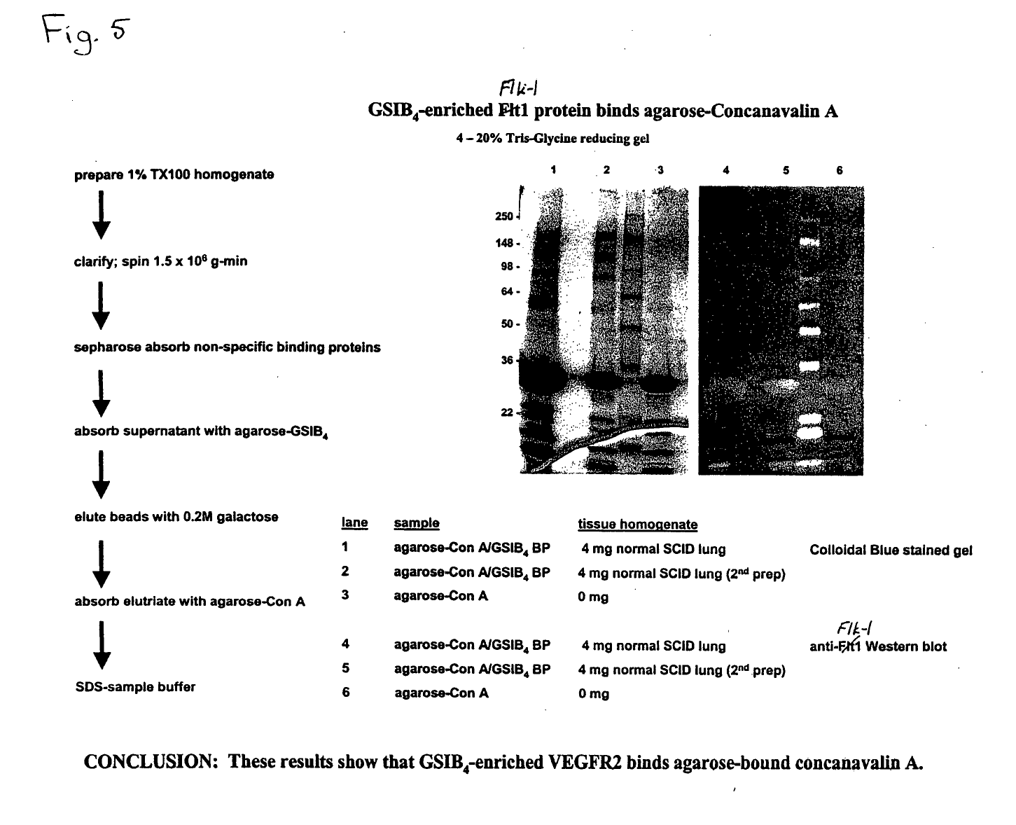

A subcutaneous human HT29 tumor xenograft was implanted in a SCID-bg mouse. Vascularized, tumor-associated and normal lung tissues were removed and tissue sections were made and labeled for staining with biotin-GSIB4 alone, or in the presence of (a) galactose, which blocks the binding of GSIB4 to the glycoproteins to which it preferentially binds, and/or (b) monoclonal anti-human EpCAM antibody, which is specific for human tissue. Sections of formalin-fixed tissue were deparaffinized, hydrated and stained 60 min with 10 μg biotin-GSIB4/ml (10 mM HEPES, 20 mM CaCl₂, 20 mM MgCl₂, Sigma). ABC reagent (30 min) and NovaRed substrate (Vector Labs) were used for color development. Staining with anti-EpCAM antibodies (2.5 μg/ml for 60 min; Dako) was detected using a VectaStain AP kit and Vector Blue substrate. Results were photographed using a Nikon E600 microscope and RT Color digital camera (Diagnostic Instruments). As shown in FIGS. 3A-3I, GSIB4 binds preferentially to cells lining blood vessel in the murine vascularized tumor-associated tissue. GSIB4 affinity purification and SDS-PAGE

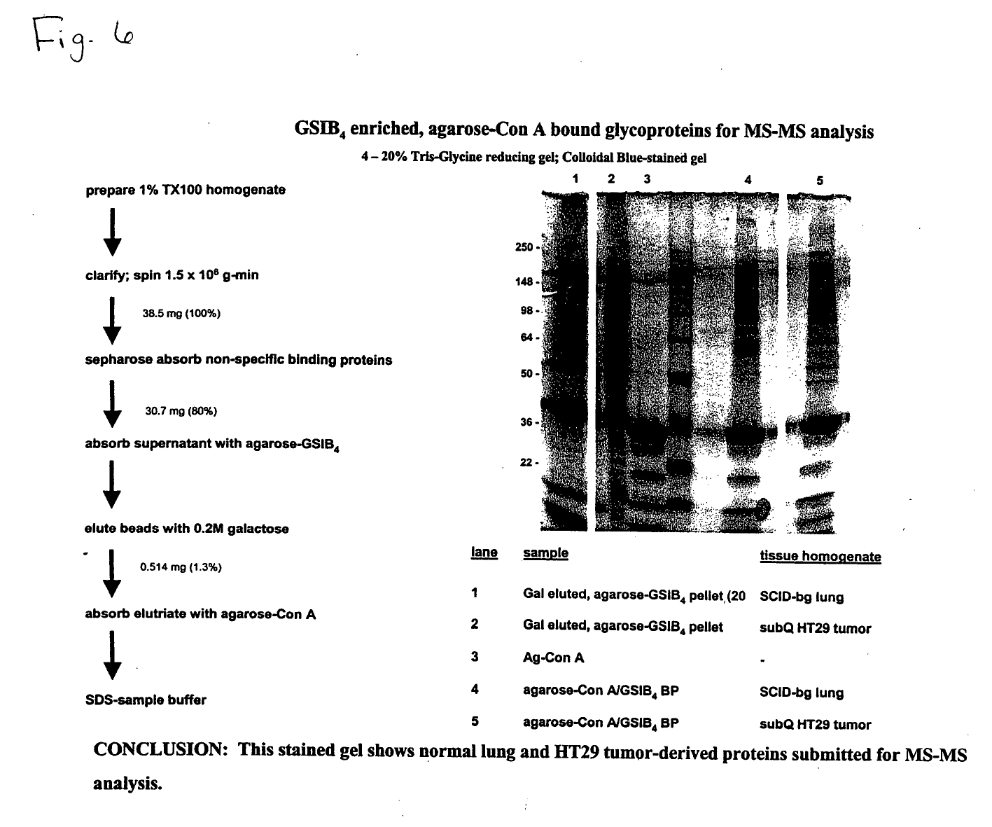

Normal lung tissue was homogenized in a solution containing 1% Triton-X-100® (alpha-[4-(1,1,3,3-tetramethylbutyl)phenyl]-omega-hydroxypoly (oxy-1,2ethanedlyl)), the solution was clarified by spinning at 1.5×10⁶ g-min, and the supernatant was contacted with Sepharose 6B® agarose (Sigma) for adsorption of non-specific binding proteins. The supernatant containing proteins that did not adsorb to the Sepharose 6B® was contacted with agarose-GSIB4, and glycoproteins that bound to the agarose-GSIB4 were eluted with sample buffer for SDS-PAGE. GSIB4 (Sigma) was coupled to agarose using AminoLink Plus Immobilization Kit (Pierce). Protein content was monitored at each purification step using Dc Protein Assay (BioRad). Eluted proteins from normal lung tissue were separated by SDS-PAGE in a 4-20% SDS Tris-glycine reducing gel. Gel lanes were stained with colloidal blue stain and a Western blot was made and probed with antibody specific for the endothelial cell-specific transmembrane protein Flk-1 (anti-Flk-1). As shown in FIG. 4, Flk-1 was successfully purified from the normal lung homogenate by affinity chromatography with agarose-GSIB4.

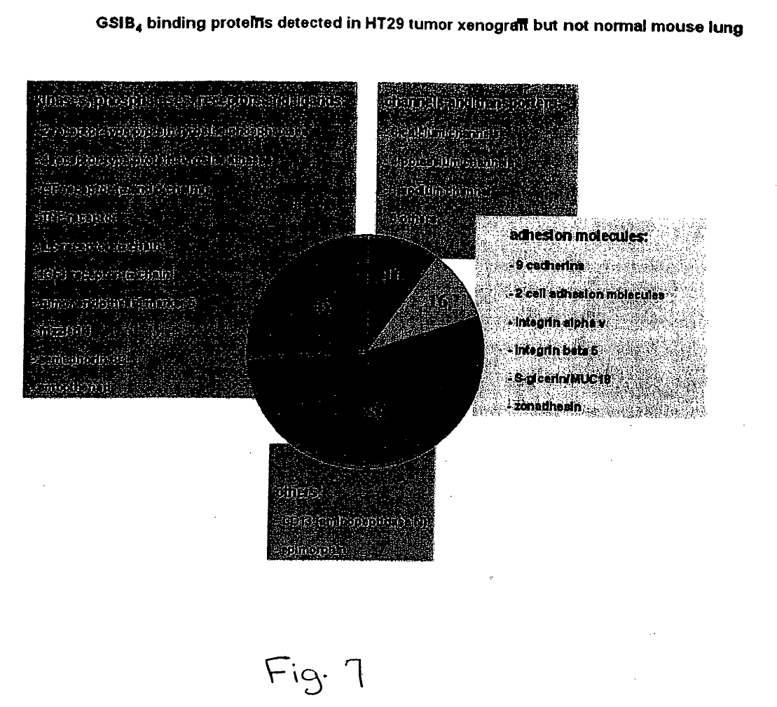

In a variant of the affinity purification procedure, Triton-X-100® homogenate of normal mouse lung was clarified, the supernatant was contacted with Sepharose® agarose to remove non-specific binding proteins, and then was contacted with agarose-GSIB4. Glycoproteins that bound to the agarose-GSIB4 were eluted with 0.2 M galactose, and then were contacted with agarose-Concanavalin A (Con A, from Sigma). Con A is a lectin that preferentially binds glycoproteins containing mannose and glucose. Glycoproteins were eluted from agarose-Con A with sample buffer for SDS-PAGE, and the eluted proteins were separated by SDS-PAGE, stained with colloidal blue stain, and analyzed by Western blot using anti-Flk-1 antibody as described above. As shown in FIG. 5, Flk-1 was also successfully purified from normal lung homogenate by affinity chromatography purification using the two lectins, GSE34 and Con A.

### Example 2

### Mass Spectrometry Microsequencing:

A. Tumor-associated and normal lung tissues were homogenized in a solution containing 1% Triton-X-100®, and the solutions were clarified by spinning at 1.5×10⁶ g-min. In a typical preparation, the clarified supernatant contained about 38.5 mg protein (100%). The clarified supernatants were contacted with Sepharose 6B® (Sigma) to remove non-specific binding proteins, after which the protein content of the supernatant was about 30.7 mg (80%). The supernatant was then contacted with agarose-GSIB4 beads, and 0.2 M galactose was used to elute 0.514 mg of glycoproteins that bound to the agarose-GSIB4 (1.3%). The glycoproteins eluted from the agarose-GSIB4 beads were contacted with agarose-Con A beads (Sigma), and then were eluted from the agarose-Con A with sample buffer for SIDS-PAGE. The proteins were electrophoretically separated on 4-20% gradient SDS gel. The gel was stained with colloidal blue stain (FIG. 6), and then de-stained prior to cutting the separation lane into 12 bands, each ⅛ thick. The proteins were digested with trypsin in situ in the gel, and the resulting peptides extracted. The peptides were loaded onto a column for separation by reversed phase high-pressure liquid chromatography (HPLC), and then were eluted into an Agilent MSD Ion Trap mass spectrometer for sequencing by tandem ion trap mass spectrometry-microsequencing (MS-MS).

LC Parameters The Reverse Phase run was 110 minutes using a 0.1% Formic Acid mobile phase and 60% Acetonitrile as the elution buffer. The gradient was 5-60% elution buffer starting at 5 min to 75 min.

The mass spectrometer was run in positive ion mode at 4000 V. The target acquisition was 30,000 ions or 300 milliseconds. The temperature was kept at 325° C. The pressure of the nebulizer gas was 25 psi and the flow of the dry gas was 12 L/min. The molecular weight scan range was 400-1800. For the MS/MS, 3 precursor ions were used with active exclusion after 1 spectra.

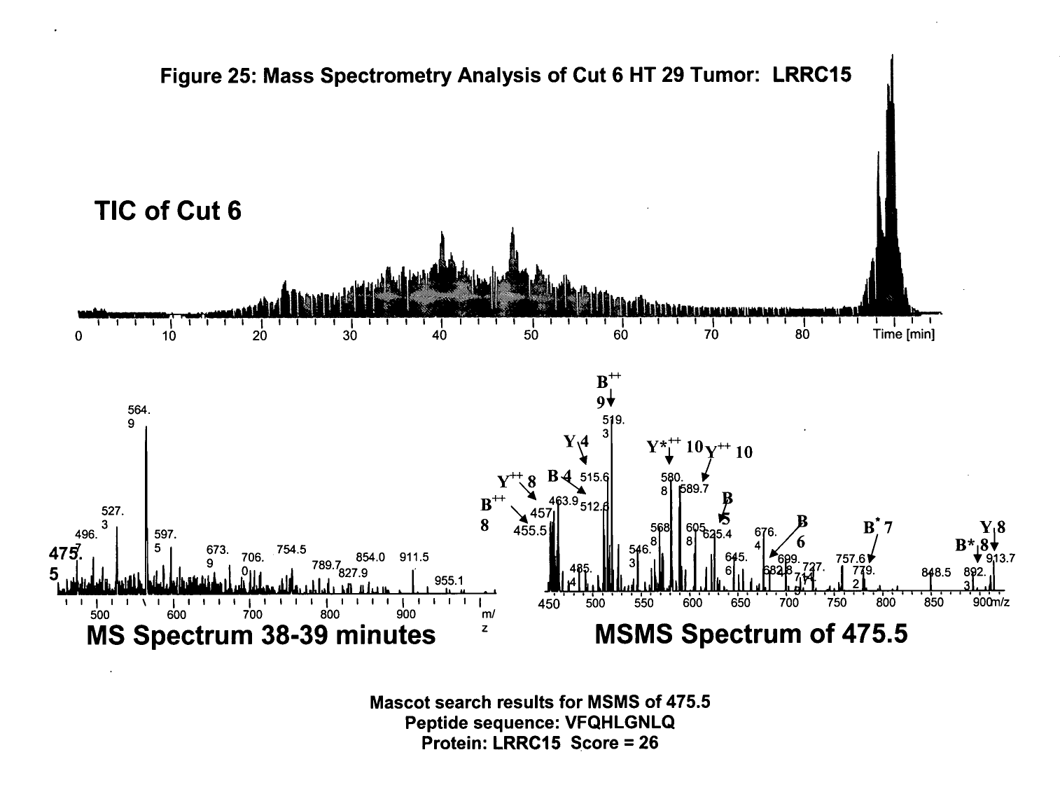

The resulting total Ion Chromatograph is shown in FIG. 25A. The MS spectrum for “Cut 6,” i.e., the sixth gel band, is shown in FIG. 25B. A smaller MS/MS spectrum, containing the data for the murine LIB protein, is shown in FIG. 25C. The ions used to identify the Lib peptide are noted.

### Example 3

### Bioinformatics

The computer program Mascot® (Matrix Science) was use to identify proteins from MS-MS data. Purified lectin-binding proteins were identified by comparing the peptide amino acid sequences determined by MS-MS to known protein amino acid sequences accessible in EMBL and Genbank databases. BLAST (Basic Local Alignment Search Tool) software was used to eliminate redundant sequences with multiple accession numbers. The results are summarized in Table 1. Of 1442 murine GSIB4-binding proteins purified from normal lung (N), only 183 (N+T) were also in the set of 1111 GSIB4-binding proteins purified from the tumor-associated vascularized tissue (T).

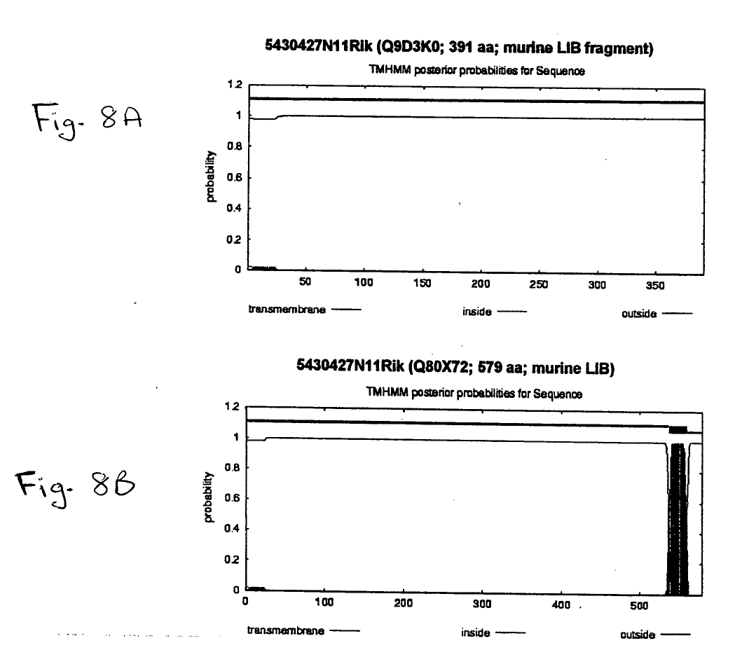

928 (84%) of the 1111 murine GSIB4-binding proteins purified from the tumor-associated vascularized tissue were specifically present (T-N) in the tumor-associated tissue; i.e., they were not found in the set of GS1134-binding proteins purified from normal lung tissue. Examples of GSIB4-binding proteins purified from normal murine lung tissue, including proteins specifically present in normal lung relative to the tumor associated vasculature (N−T), and proteins that were detected in both normal lung and tumor-associated tissue (T+N), are listed in Tables 2a and 2b. Examples of GSIB4-binding proteins that were specifically present in the tumor-associated tissue (T−N) are listed in Tables 3a and 3b. Of the GSIB4-binding proteins that were specifically present in the tumor-associated tissue, 160 had transmembrane (TM) domains. As shown in FIG. 7, 16 (10%) of these TM proteins were channels and transporters, 16 (10%) were cell adhesion molecules, 42 (26%) were receptors and ligands, and 86 (54%) were other types of proteins.

Tables 4a-4c present another list of (T−N) murine GSIB4-binding proteins that were identified in HT29 tumor xenograft vasculature but not normal mouse lung samples. The murine GSIB4-binding proteins listed in Table 4a have human homologs for which little data is available regarding their expression in somatic tissues. Among these proteins are the murine homologs of the human CDO, TMEFF2, KIAA1484, LRRN3, LRRC15, and Slit-like 2 protein, which are transmembrane proteins identified by the invention as targets for anti-tumor agents that inhibitor disrupt tumor angiogenesis.

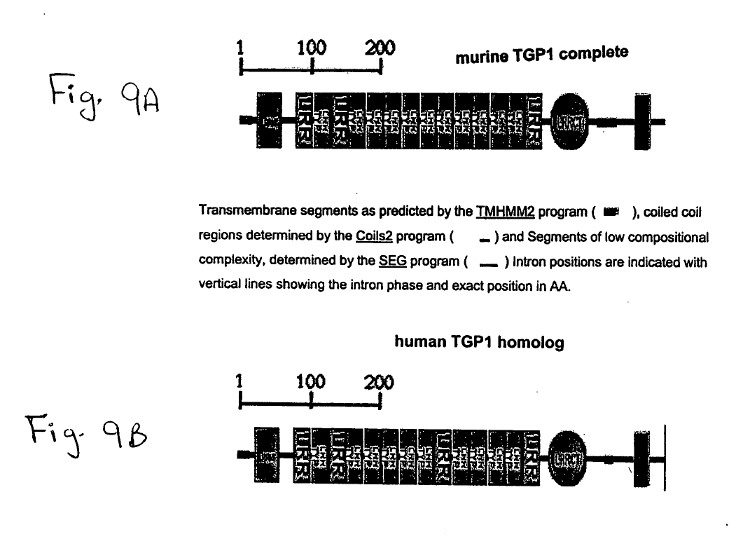

Predicted structural features of the 391 amino acid murine LIB fragment, and the 579 amino acid murine LIB protein that were identified as GSIB4-binding proteins are shown graphically in FIGS. 8A and 8B, respectively. Schematic representations of predicted structural features of the full length murine LIB protein and the homologous human LRRC15 protein are shown in FIGS. 9A and 9B, respectively. The nucleotide sequence encoding LRRC15 (SEQ ID NO:1) is shown in FIG. 1A, and the amino acid sequence is shown in FIG. 1B (SEQ ID NO:2).

### Example 4

### Expression of Human Homologs of Murine GSIB4-Binding Proteins in Human Tissues

RT-PCR amplification of mRNA directed by the human LRRC15 gene (hTGP1) and the human Slit-like 2 gene (hTGP2) in human cells in vitro and in normal and tumor tissues was detected by RT-PCR, and the DNA products of RT-PCR were visualized in agarose gels.

A. Expression of the Human LRRC15 Gene:

The PCR primers used for detecting human LRRC15 gene expression are shown below. These primers amplify a PCR product of 391 bp from any cDNA template containing the Lib gene. Since human ESTs are not commercially available, a synthetic positive control was made by adding the primers to a DNA fragment.

5′GACGGGAATGTCTTCCGCATGTTGG 3′(SEQ ID NO:6)
5′TTTCTGGGTAACTAGGCACCTCAGG 3′(SEQ ID NO:7)

The artificial positive control generates a 350 bp band. Expression of Glyceraldehyde 3-phosphate dehydrogenase (GAPDH) is measured in all experiments as a control for cDNA integrity. GAPDH is a housekeeping gene expressed abundantly in all human tissues. Primers used for amplification of the GAPDH gene are:

5′-ACCACAGTCCATGCCATCAC-3′(SEQ ID NO:8)
5′-TCCACCACCCTGTTGCTGTA-3′(SEQ ID NO:9)

These primers amplify a 482 bp product from any cDNA template encoding the GAPDH gene. In all cases, positive and negative controls are also included; the positive control is the synthetic fragment containing the Lib specific primers, the negative control is water (no template).

The in-house tumor samples were obtained from Grossmont Hospitals. The tumor cell lines were provided by Francesca and Edwin at Cancer Therapeutics Department, who obtained the cell lines originally from The Arizona Cancer Center (UACC812) and ATCC (the rest of the cell lines). Normal cell lines were provided by Julia Coronella at Antibody Discovery who obtained the cell lines from Cambrex. RNA was isolated from each tumor sample and cell line using Qiagen's RNeasy kit (catalog # 75162). cDNA was prepared from total RNA using Invitrogen's cDNA synthesis system (catalog # 11904-018.) In each of the following figures, the upper panel shows LRRC15 expression, the lower panel shows GAPDH expression.

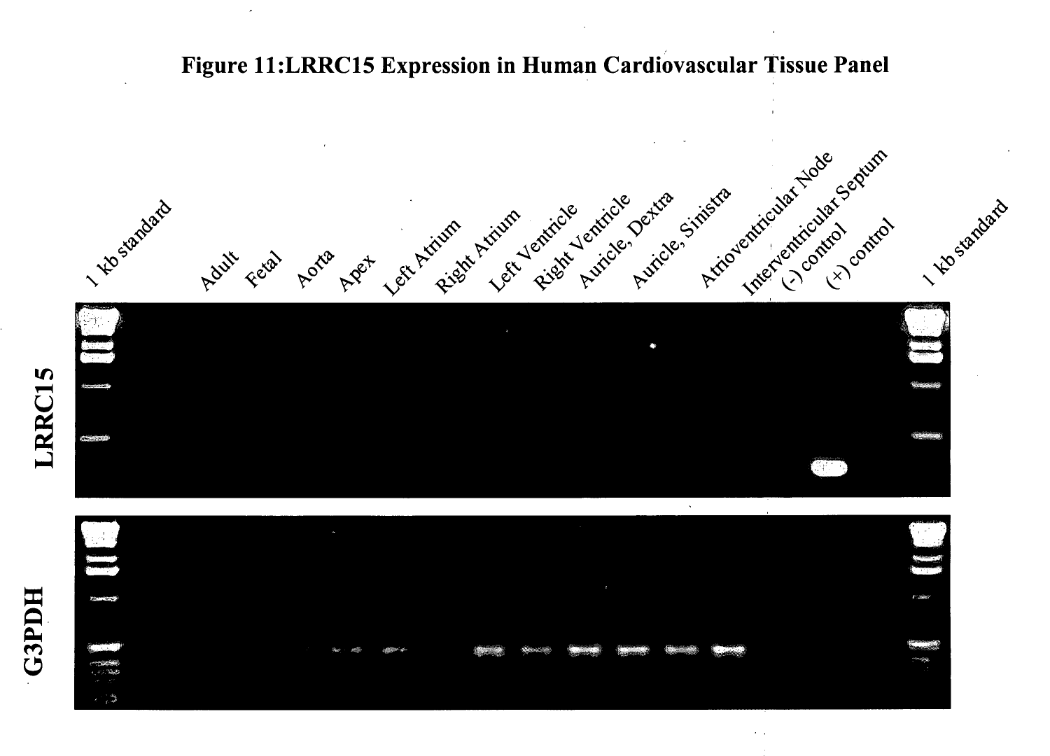

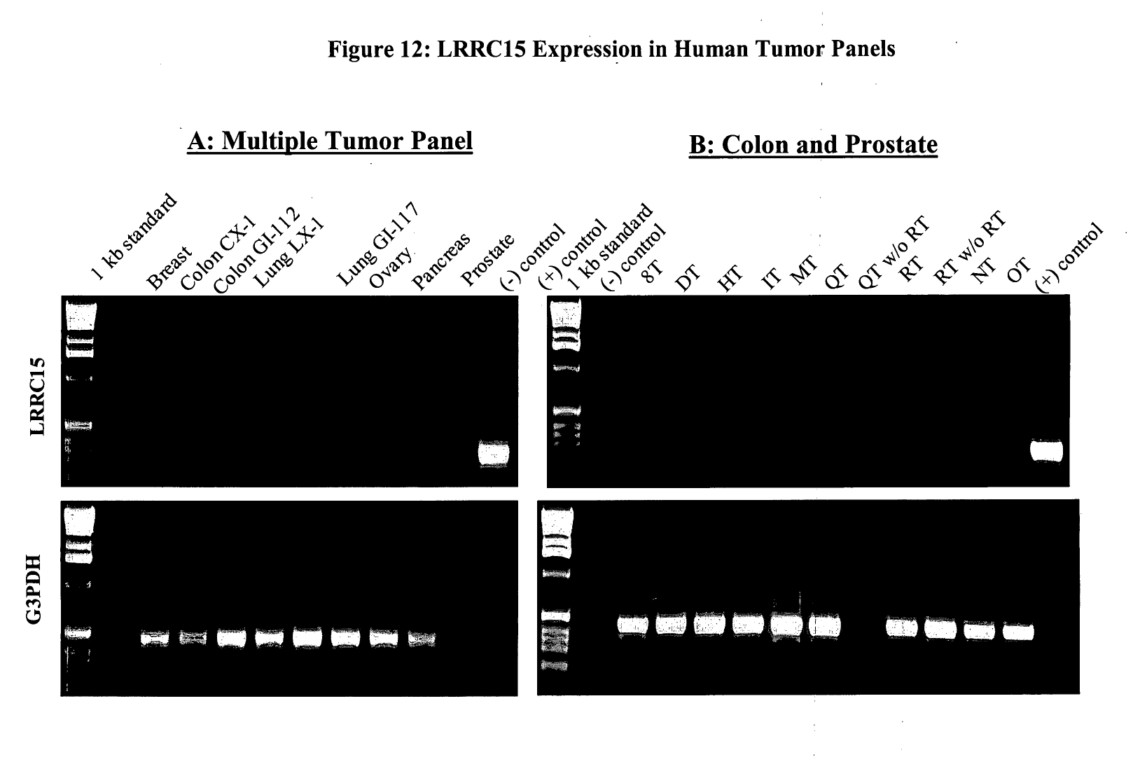

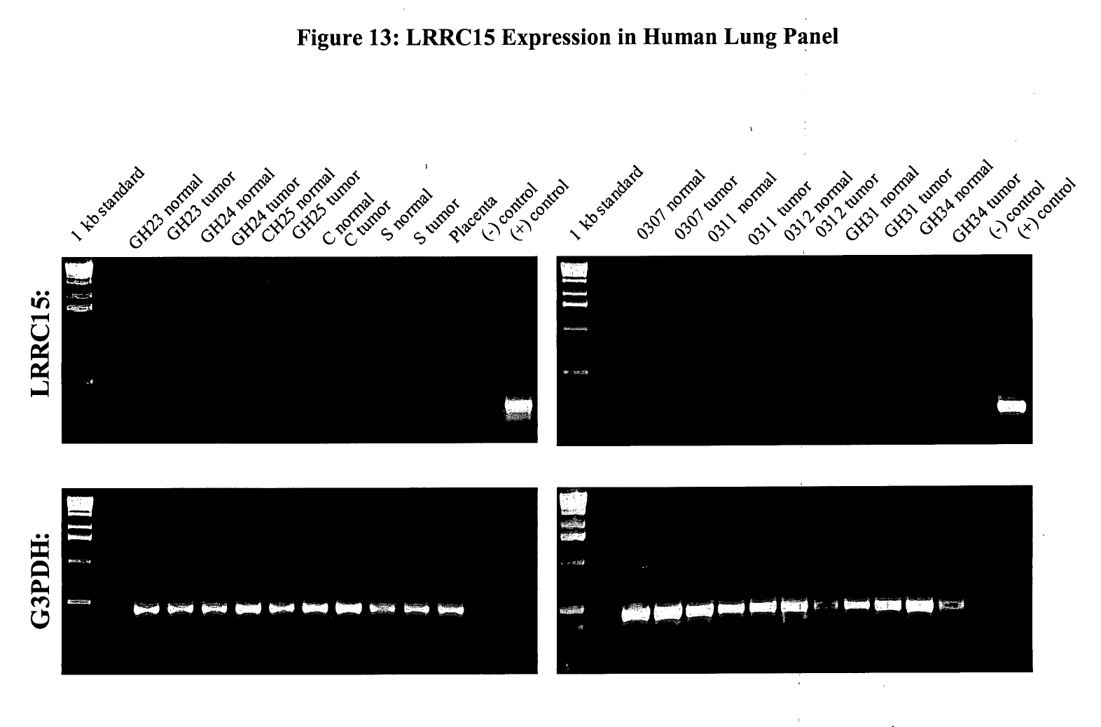

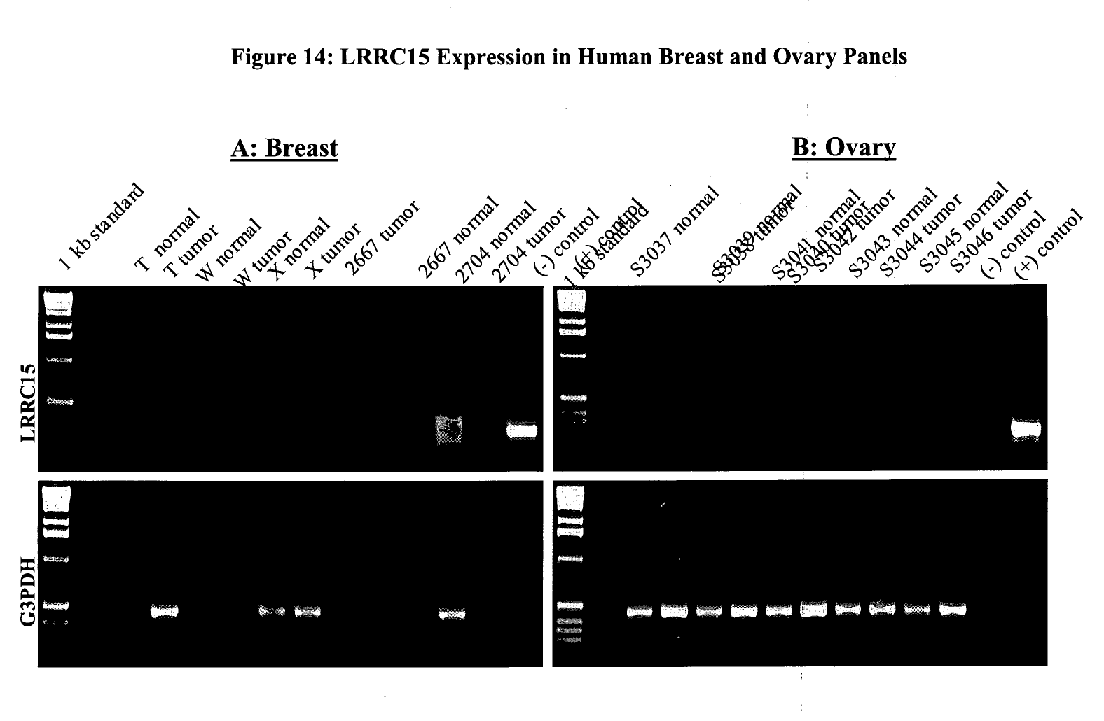

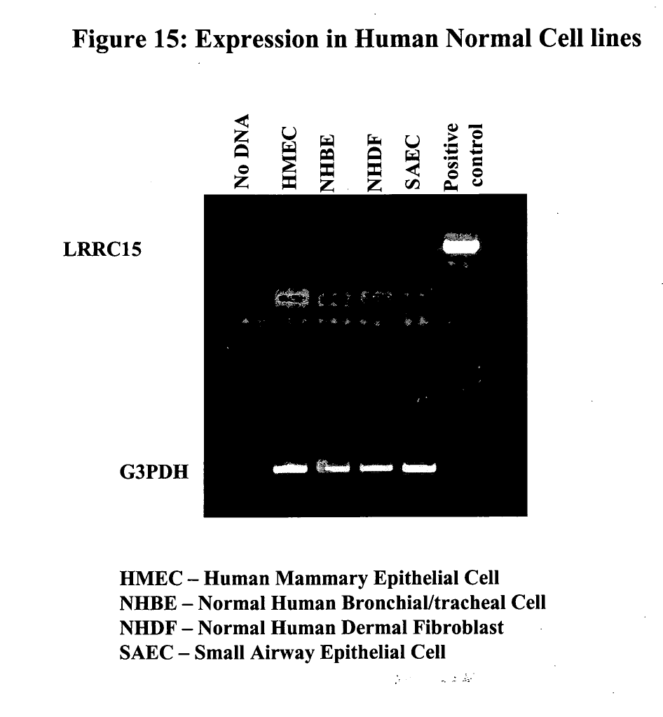

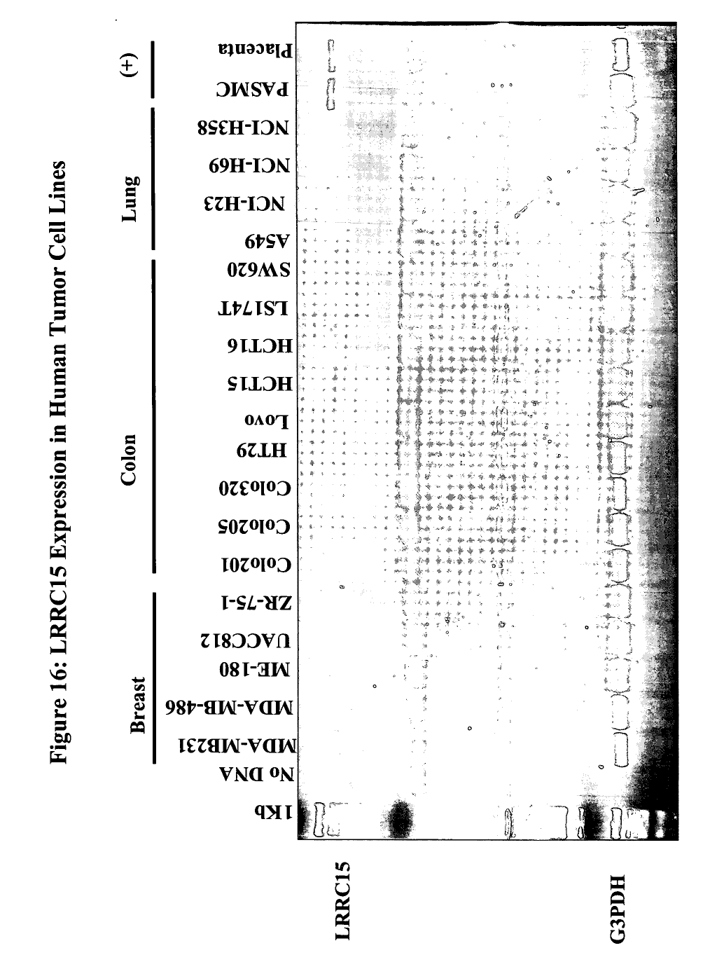

The expression of Lib in normal and malignant human tissues was investigated by PCR experiments using commercially available human cDNA panels and cDNA samples prepared in-house from human tissues and cell lines. FIG. 10 shows LRRC15 expression in human multiple tissue panels I and II. The cDNAs were purchased from BD Clontech (catalog # K1423-1 and K1430-1). As can be seen, all tissues were negative for LRRC15 expression except for placenta. FIG. 11 shows LRRC15 expression in a human cardiovascular MTC tissue panel (BD Clontech catalog # K1427-1). All tissues were negative for LRRC expression. FIG. 12A shows LRRC15 expression in a multiple tumor tissue panel (BD Clontech catalog # K1422-1). In this panel, all samples were negative for LRRC15 expression. FIG. 12B shows LRRC15 expression in an in-house-derived panel of colon and prostate tumors. In this panel, all tissues except for 8T and DT (lanes 3 and 4) were positive ror LRRC15 expression. FIG. 13 shows LRRC15 expression in an in-house-derived panel of lung tumor/normal tissue matched pairs. In this experiment, one tumor tissue (0307T, lane 3 in the second panel) was positive, and one normal tissue (0312N, lane 8 in the second panel) was positive. FIGS. 14A and B show LRRC15 expression in an in-house-derived panel of breast (panel A) and ovarian (panel B) tumor/normal tissue matched pairs. In the breast panel, all of the tumor tissues except 2667 T were positive for LRRC expression, while all of the normal tissues were negative. In the ovary panel, three of five tumor tissues were positive for LRRC expression, where all of the normal tissues were negative. FIG. 15 shows LRRC15 expression in normal cell lines, all of which were positive. FIG. 16 shows LRRC15 expression in tumor cell lines. Three of five breast tumor lines were positive, one of 8 colon tumor lines were positive, and 4 of 4 lung tissue lines were positive.

B. Expression of the Human Slit-Like 2 Gene:

The PCR primers used for detecting human Slit-Like 2 gene expression are shown below:

5′ACAGACAGTCTTCTGCACTGC 3′(SEQ ID NO:10)
5′TCGTTGTCCTGCAGCTTGAGC 3′(SEQ ID NO: 11)

In vitro cell expression of the human Slit-like 2 gene is elevated in human vascular smooth muscle cells (HVSMC) but not in PBLs, endothelial cells or HT29 colon tumor cells. Expression of the human Slit-like 2 gene is detected in diverse normal and tumor tissues. See FIG. 17.

### Example 5

### UEA1 Histochemistry

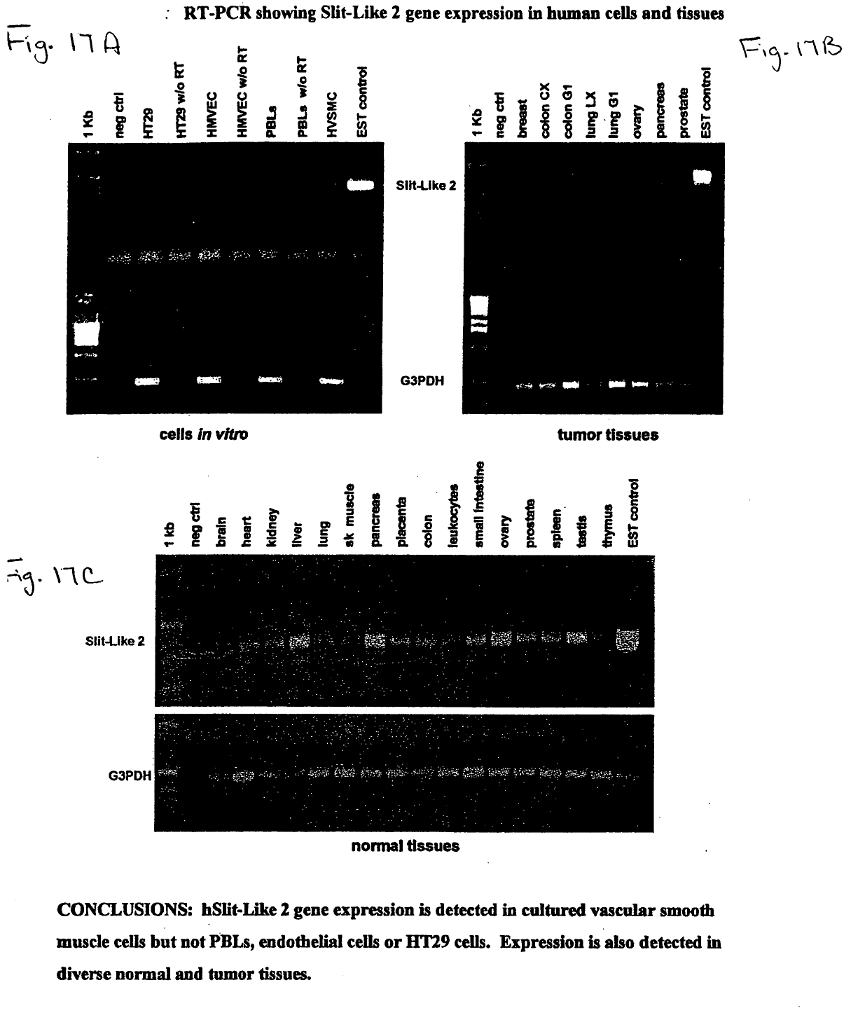

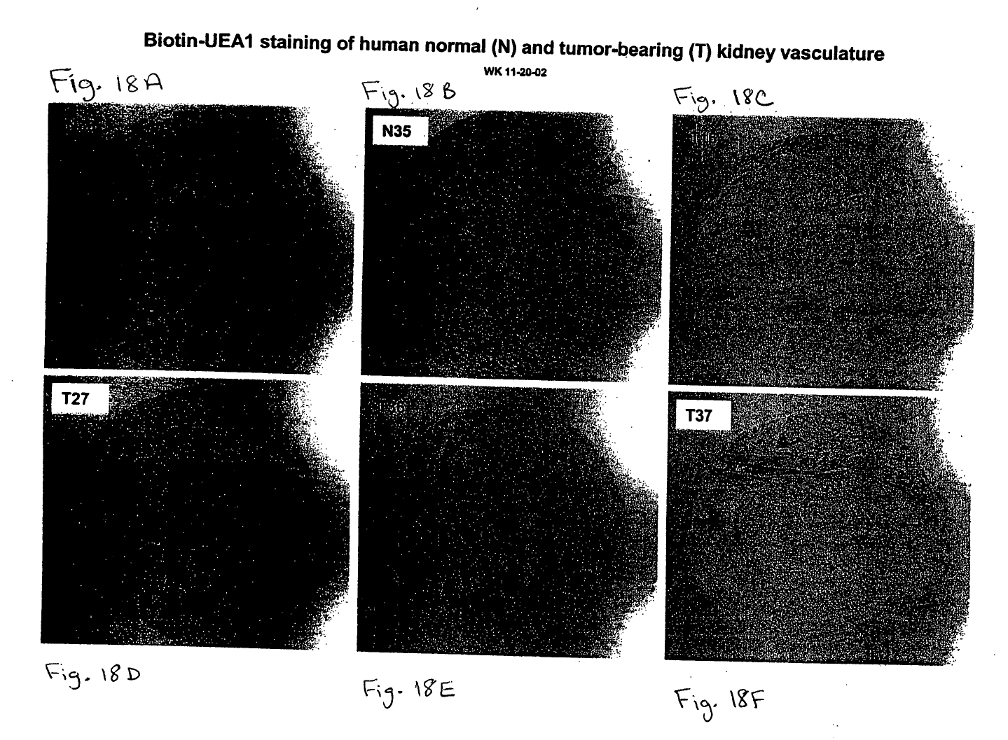

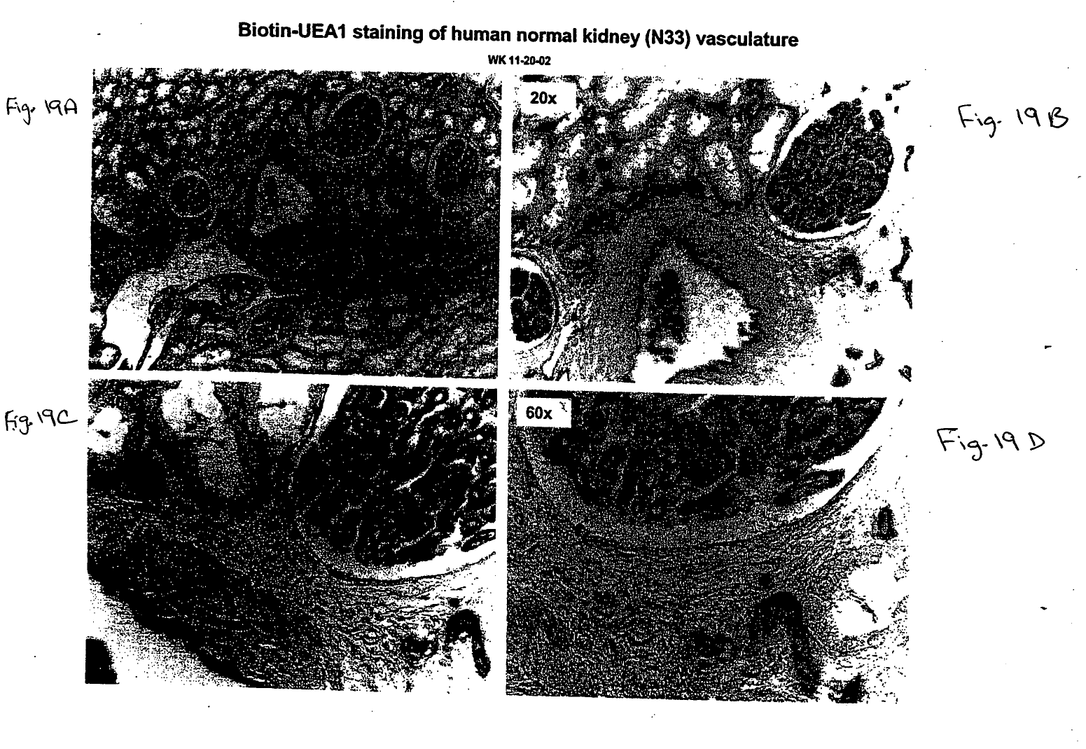

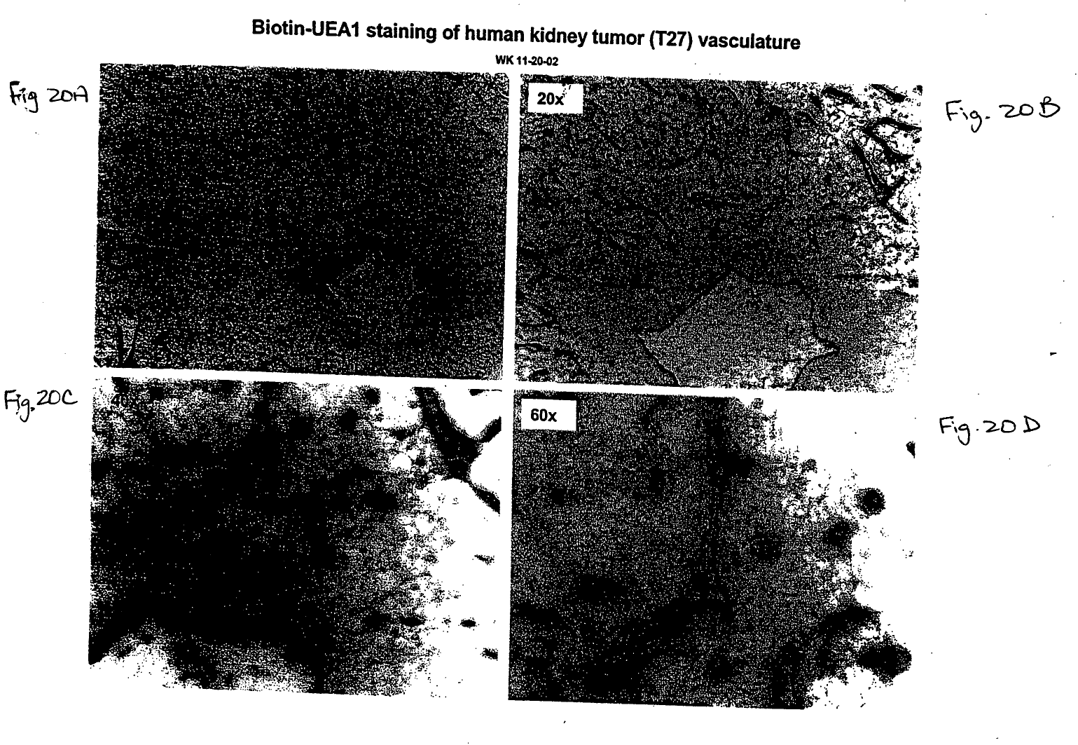

Human normal and tumor-bearing kidney tissues were sectioned and fixed, stained with biotin-UEA1, and photographed as described above for staining murine tissues with GS1134. FIGS. 18A and 18B show that biotin-UEA1 selectively stains glomeruli and other renal vasculature of two human normal kidney tissues, N33 and N35. FIGS. 18C-18F show that biotin-UEA1 also selectively stains kidney tumor vasculature of four different human tumor-bearing kidney tissues, T16, T27, T36, and T37. Staining of glomeruli and other renal vascular structures in human normal kidney tissue by UEA1 is shown at successively higher magnifications in FIGS. 19A-19D. Similarly, staining of human kidney tumor vasculature by UEA1 is shown at successively higher magnifications in FIGS. 20A-20D.

### Example 6

### Cloning and Expression of the LRRC15

A. Full-Length LRRC15 Gene

A cDNA clone encoding the 581-amino acid LRRC15 protein was amplified by PCR from the breast cancer cell line ME-180 with desired restriction sites. The 5′ forward primer was as follows: 5′ ACTACTGCTA GCTTAACACT CATTGGGTGC CTTCATCTGC 3′ (SEQ ID NO:12), the 3′ reverse primer was as follows: 5′ ACTACTGTCG ACCTCACCAT GCCACTGAAG CATTATCTCC TTTGC 3′ (SEQ ID NO:13).

After standard amplification, the PCR product was directly inserted into the PCR cloning vector pGEM-T-Easy, using the system available from Promega (catalog # A1360). The proper insert was confirmed by nucleotide sequencing. Full-length LRRC15 gene was also cloned into N5K-Cidectin-B71g expression vector for further study.

B. LRRC15 Extracellular Domain-Fc Fusion Construct

In this example, a soluble fusion polypeptide was constructed, comprising the extracellular domain of LRRC15, extending from amino acid 22 to amino acid 537 of SEQ ID NO:2 (FIG. 1), fused at its C-terminus to a human IgG1 Fc domain. In addition, construct was prepared with an internal sequence encoding the TEV protease site (ENLYFQG, SEQ ID NO:14) between Lib and Fc providing the option to proteolytically cleave and release the Lib extracellular domain (Invitrogen, Carlsbad, Calif., USA).

The fusion construct was made as follows. DNA fragments encoding the amino terminal and carboxyl terminal portions of the extracellular domain of LRRC15 were amplified by PCR from the full-length gene sequence (SEQ ID NO:1, FIG. 1A). The sequence encoding the extracellular domain was amplified in two sections to facilitate use of certain restriction endonuclease sites. Primers used for amplifying LRRC15 amino terminal extracellular domain fragment were: P1 forward primer 5′ GCGGCGAGAT CTCTCACCAT GCCACTGAAG CATTATCTCC 3′ (SEQ ID NO:15) and P2 reverse primer 5′ GAGAGACTCG AGGCTTGTCCA 3′ (SEQ ID NO:16). Primers used for amplifying LRRC15 carboxyl terminal extracellular domain fragment were: P3 forward primer 5′CAACCAGCCT AGGTTAGGG 3′ (SEQ ID NO:17) and P4 reverse primer 5′ GCGGCGGCTA GCGCCCTGGA AGTACAGGTTC TCGCTCTGGG CCTGGGTCAT GCCCC 3′(SEQ ID NO:18), the underlined sequences in the P4 reverse primer coding for the TEV protease cleavage site.

PCR was performed using standard conditions, the resulting products were isolated by standard methods and the amino and carboxyl terminal DNA fragments were digested with BglII/XhoI and AvrII/NheI, respectively. The full length LRRC15 DNA vector, prepared as described in part A above, was digested with XhoI and AvrII and the internal XhoI/AvrII fragment isolated. The amino terminal BglII/XhoI and the internal XhoI/AvrII DNA fragments were then ligated into a BglII/AvrII digested mammalian expression vector containing human IgG1 Fc cDNA sequence. This intermediate vector was digested with AvrII/NheI and ligated to the carboxyl terminal AvrII/NheI DNA fragment to complete the nucleotide sequence encoding the fusion protein. The predicted sequence of the construct was verified by nucleic acid sequencing. The DNA (SEQ ID NO:3) and amino acid (SEQ ID NO:4) sequences of the fusion construct are shown in FIGS. 2A and 2B, respectively.

C. Construction of Stable Cell Lines Expressing LRRC15

The full length and fusion constructs described above were subsequently used to generate stably transfected Chinese hamster ovary (CHO) cell lines. Briefly, expression constructs were transfected into DHFR-CHO DG44 cells (Urlaub et. al., Som. Cell. Mol. Gen. 12:555-566 (1985)) by electroporation. Cells were washed, counted and resuspended in ice cold PBS buffer (2.67 mM KCl, 1.47 mM KH2PO4, 137.93 mM NaCl, 8.06 mM Na2HPO4-7H20). Plasmid DNA was linearized by PacI restriction digestion and 2, 1 or 0.5 ug/ml DNA mixed with 4×106 DG44 cells and electroporated. Cells were seeded into 96-well microtiter culture plates and cell lines were selected for G418 resistance in CHO S SFM II media (Gibco) supplemented with hypoxanthine+thymidine (HT, Gibco).

G418 resistant cell lines expressing full-length Lib from plates transfected with the lowest concentration of DNA and exhibiting robust cellular growth were screened by B7Ig surrogate marker expression by ELISA. Briefly, Immulon II plates (Thermo Labsystems) were coated with goat anti-human IgG and nonspecific sites blocked. Supernatants from G418 resistant cell lines were diluted into binding buffer (0.5% non-fat milk in PBS) and added to the plates. Captured immune complexes were detected by incubating with HRP-conjugated goat anti-human IgG (Southern Biotechnology) and developed with TMB Peroxidase substrate (KPL Inc.) Color development was quenched by the addition of 2N H2SO₄, and absorbence were measured using a microtiter pate reader (Molecular Dynamics) at a dual wavelength setting of 450/540 nm. The cell lines secreting the highest levels of B7Ig were further analyzed for LRRC15 mRNA expression by RT-PCR. Total RNA from 5×10⁶ cells of the top six highest B7Ig producing clones was extracted using Qiagen RNeasy® Mini kit following the manufacturer's protocol. cDNA was synthesized using Invitrogen Super Script First Strand Synthesis System for PCR according to manufacturer's protocol. The following primers were synthesized and used to perform PCR.

5′ATGCCACTGAAGCATTATCT 3′(SEQ ID NO:19)
5′GCTCCGTCAGGGATGTATTC 3′(SEQ ID NO:20)

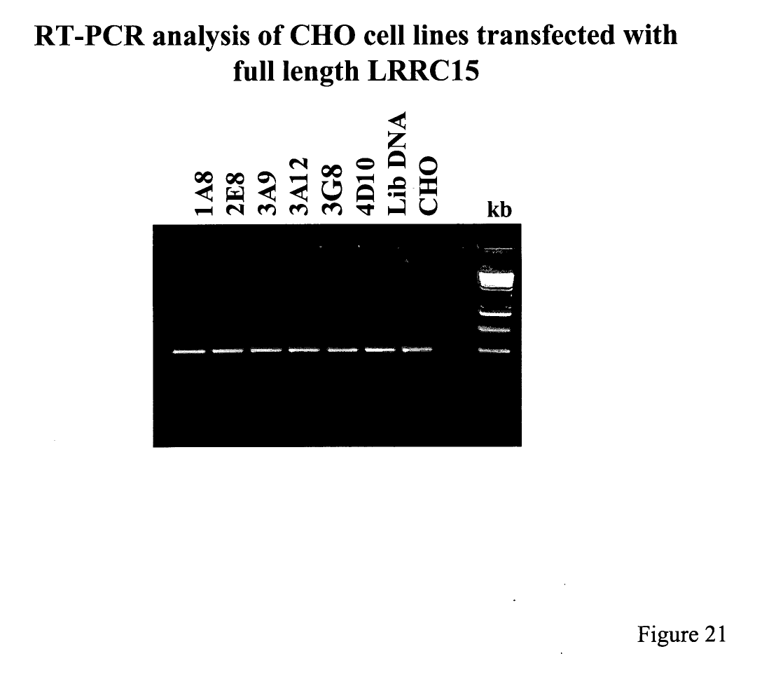

Bands were visualized on a 1.2% agarose+EtBr gel (FIG. 21). The data presented in this figure confirms that recombinant LRRC15 molecules were expressed at detectable RNA levels in mammalian cells and produce PCR products of the expected size. The top three B7Ig+ and LIB RT-PCR+CHO cell lines were amplified in 5 nM methotrexate (MTX). Briefly, cells were seeded at a density ranging from 1.5 cells/plate to 3000 cells/plate in two-fold increments and cultured in media containing 5 nM MTX for two weeks. Surviving cell lines were screened for B7Ig surrogate marker secretion by ELISA and the highest producing clones selected.

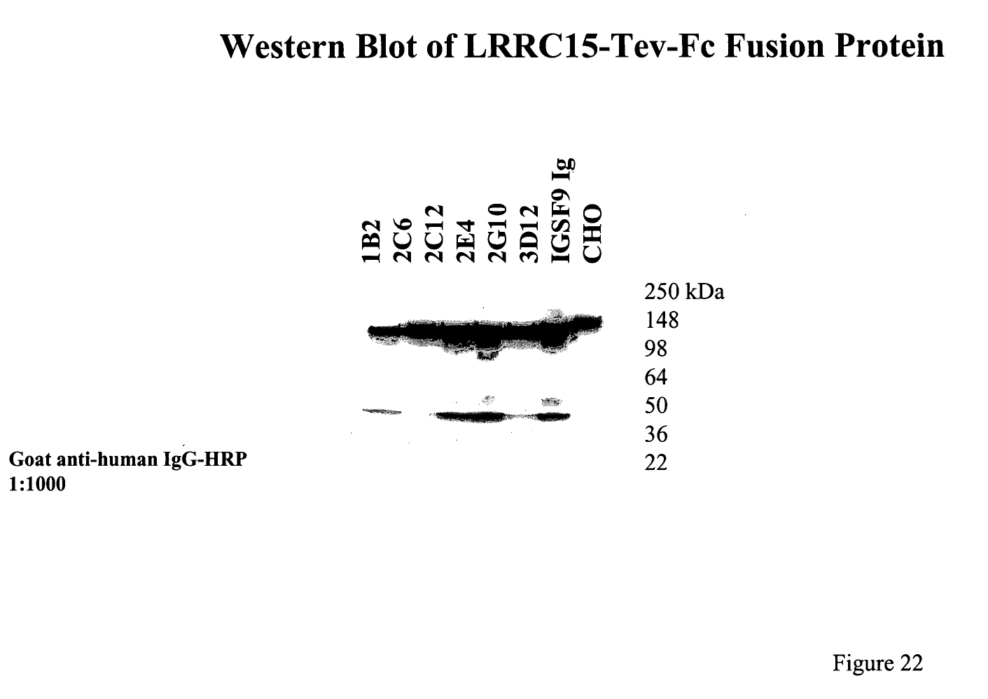

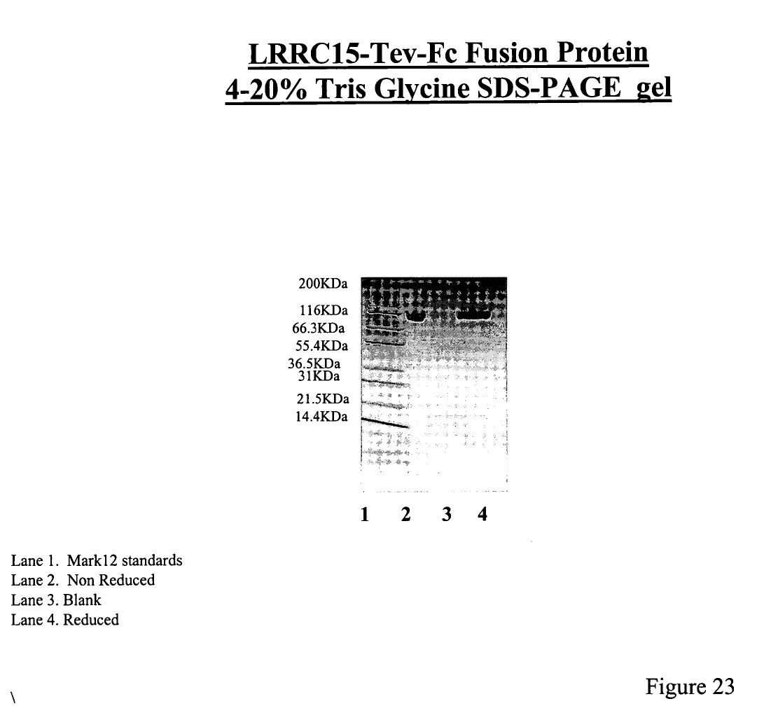

G418 resistant cell lines expressing LRRC15-Fc fusion protein from plates transfected with the lowest concentration of DNA and exhibiting robust cellular growth were identified by screening for secreted LRRC15-Fc fusion protein by ELISA. Cell culture supernatants were assayed by Western Blot from the top six highest LRRC15-Fc fusion protein producing clones. Supernatants were mixed 1:1 in 2×SDS Sample Buffer (Invitrogen) containing 2-mercaptoethanol and resolved on a 4-20% Tris-Glycine SDS gel. The proteins were then transferred to a nitrocellulose membrane. The membrane was probed with a horseradish peroxidase (HRP) conjugated goat anti-human IgG (Southern Biotechnology) at a 1:1000 dilution. Following extensive washing the membrane was developed using ECL reagent (Amersham Pharmacia) according to manufacturer's instructions. Results shown in FIG. 22 indicate that clones 2E4 and 3D12 are among the highest producers. Cell line 3D12 was expanded into a 5 liter spinner culture and Lib Fc fusion protein purified from culture supernatant by protein A affinity chromatography. FIG. 23 shows an SDS-PAGE analysis of purified Lib Fc fusion protein. A band of approximately 116 kDa, consistent with the predicted MW of the fusion protein, was seen. This data indicates that recombinant Lib molecules can be expressed successfully at high levels in mammalian cells.

### Example 7

### Production of anti-LRRC15 Monoclonal Antibodies

Monoclonal antibodies were produced by injecting groups of 6-8 week old male balb/c mice thirteen days prior to fusion with peptides derived from the extracellular domain of LRRC15. Peptide One: (N(acetylated)-CLESLLLSSNQLLQIQPAHFSQSSNLKELQLH-COO-(acid)), designated herein as SEQ ID NO:21, has 32 amino acids, with amino acids 2 to 32 being identical to amino acids 127 to 157 of SEQ ID NO:2. Peptide Two: (NH2(amino)-EVPSYPETPWYPDTPSYPDTTSVSSTTELTC-COO (amide)), designated herein as SEQ ID NO:22, has 32 amino acids with amino acids 3 to 32 being identical to amino acids 480 to 509 of SEQ ID NO:2. The peptides were coupled to ovalbumin for use in the immunizations. Two groups of 5 mice were immunized with each of the coupled peptides using 20 μg protein/per mouse in RIBI adjuvant, and 20 μg protein/mouse in complete Freund's adjuvant, in a rapid immunization technique consisting of five sets of twelve injections over a period of eleven days.

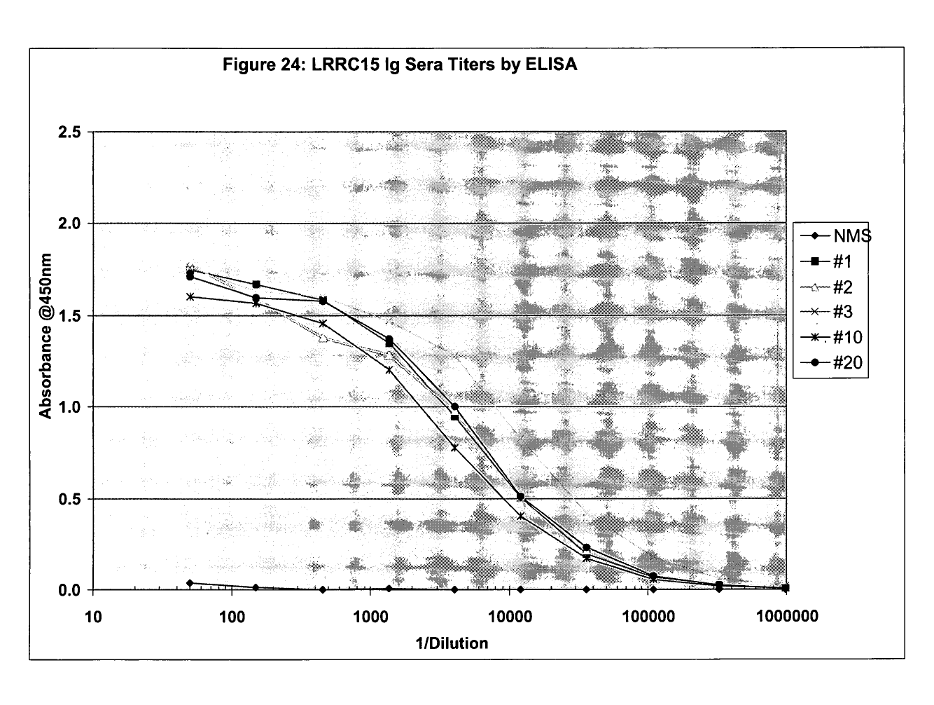

The immunized mice were bled by standard techniques, and serum titers were measured by ELISA on microtiter plates coated with the same peptides. Serum titers for individual mice, compared with a control of normal mouse serum (“NMS”) are shown in FIG. 24.

PEG-induced cell fusions were then carried out on spleen cells from mice with the highest titers to form hybridomas. All hybridomas were also initially screened by ELISA for binding to the peptides used for immunization, and all positives from the first screen were then tested on irrelevant negative control peptides to rule out any non-specific antibodies prior to subcloning by limiting dilution. Hybridomas which secrete antibodies which specifically bind to the LRRC15 peptides are subjected to subcloning and limiting dilution in order to isolate hybridoma clones which produce LRRC15-specific monoclonal antibodies. Complementary DNAs encoding the cloned monoclonal antibodies are isolated and subjected to nucleotide sequencing. and host cells stably expressing the desired antibodies are constructed. Large quantities of the desired antibodies are then produced either from the host cells or in mouse ascites, and the antibodies are subjected to further characterization, for example assays to test for the ability of the antibodies to retard the growth of cultured tumor cells in vitro. The cDNAs encoding suitable monoclonal antibodies are then subjected to further engineering to improve their characteristics for use an a therapeutic in humans. For example, the antibodies are subjected to humanization, affinity maturation, chimerization, primatization, as well as other manipulations described herein.

Further immunizations were then carried out using the LRRC15-Ig fusion protein produced as described in Example 6B.

Briefly, monoclonal antibodies are produced by injecting groups of 6-8 week old male balb/c mice thirteen days prior to fusion with the LRRC15 Ig fusion protein. Groups of 5 mice were immunized using 20 μg protein/per mouse in RIBI adjuvant, and 20 μg protein/mouse in complete Freund's adjuvant, in a rapid immunization technique consisting of five sets of twelve injections over a period of eleven days. The immunized mice are bled by standard techniques, and serum titers are measured by ELISA on microtiter plates coated with the same peptides.

PEG-induced cell fusions are then carried out on spleen cells from mice with the highest titers to form hybridomas. All hybridomas are initially screened on by ELISA for binding to the LRRC15-Ig fusion protein used for immunization. All positives from the first screen are then tested on irrelevant negative control peptides to rule out non-specific antibodies prior to subcloning by limiting dilution. Hybridomas which secrete antibodies which specifically bind to the LRRC15-Ig fusion protein are subjected to subcloning and limiting dilution in order to isolate hybridoma clones which produce LRRC15-specific monoclonal antibodies. Complementary DNAs encoding the cloned monoclonal antibodies are isolated and subjected to nucleotide sequencing. and host cells stably expressing the desired antibodies are constructed. Large quantities of the desired antibodies are then produced either from the host cells or in mouse ascites, and the antibodies are subjected to further characterization, for example assays to test for the ability of the antibodies to retard the growth of cultured tumor cells in vitro. The cDNAs encoding suitable monoclonal antibodies are then subjected to further engineering to improve their characteristics for use an a therapeutic in humans. For example, the antibodies are subjected to humanization, affinity maturation, chimerization, primatization, as well as other manipulations described herein.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

TABLE 1
GSIB₄ binding proteins identified from normal mouse lung and HT29 tumor xenograft
Oct. 02, 2003

| | normal | | | | | | | HT29 tumor |
|---|---|---|---|---|---|---|---|---|
| | lung (N) | N − T | | T + N | | T − N | | xenograft (T) |
| alignments | 2581 | — | | — | | — | | 1740 |
| non-redundant alignments | 1442 | 1259 | | 183 | | 928 | (84%) | 1111 |
| proteins with TM domains | | 302 | (24%) | 41 | (22%) | 146 | (16%) | |
| proteins lacking TM domains | | 957 | | 142 | | 768 | | |
| >truncated TM proteins | | — | | — | | 14 | | |
| total TM (TMHMM) proteins | | 302 | | 41 | | 160 | | |
| >transporters & channels | | 32 | (11%) | 2 | (5%) | 16 | (10%) | |
| >cell adhesion molecules [symbol] | | 16 | (5%) | 11 | (27%) | 16 | (10%) | |
| >receptors & ligands [symbol] | | 76 | (25%) | 15 | (37%) | 42 | (26%) | |
| >others ( [symbol] b) | | 178 | (59%) | 13 | (31%) | 86 | (54%) | |

¹ cut-off scores of 15 (multiple peptides) and 25 (single peptides)

FIG. 7. Murine GSIB₄ binding proteins that have
human homologs expressed by vasculature

| | Normal | HT29 Tumor |
|---|---|---|
| GSIB₄ Binding Protein | Lung | Xenograft |
| advanced glycosylation end-products receptor | x | |
| AN2/NG2 proteoglycan | x | |
| CD31 (platelet-endothelial cell adhesion | x | |
| molecule-1) | | |
| CD34 (hematopoietic stem cell antigen) | x | |
| CD36 (platelet gpIV, TSP-1 receptor) | x | |
| CD105 (endoglin) | x | |
| EC selective adhesion molecule | x | |
| endothelin converting enzyme | x | |
| ephrin B2 (hepatoma TM kinase ligand) | x | |
| ICAM-1 | x | |
| IL-11 receptor alpha chain | x | |
| IL-13 receptor alpha chain | x | |
| integrin alpha 1 | x | |
| integrin alpha 4 | x | |
| integrin alpha 9 | x | |
| MECA32 | x | |
| natriuretic peptide clearance receptor | x | |
| protein tyrosine kinase, Eph A2 (Sek2) | x | |
| protein tyrosine kinase, Ephrin-B4 | x | |
| protein tyrosine kinase, ErbB4 | x | |
| protein tyrosine kinase, FGF receptor 4 | x | |
| protein tyrosine kinase, Flk-1 | x | |
| protein tyrosine kinase, Flk-2 | x | |
| protein tyrosine kinase, Kit | x | |
| protein tyrosine kinase, Ryk | x | |
| protein tyrosine kinase, Tie-2 | x | |
| thrombomodulin | x | |
| anglotensin converting factor | x | x |
| (peptidyl dipeptidase A) | | |
| C1Q receptor | x | x |
| cadherin-VE | x | x |
| calnexin | x | x |
| CD91 (alpha-2-macroglobulin receptor) | x | x |
| Fc gamma Rllb | x | x |
| integrin alpha 2 | x | x |
| integrin alpha 3 | x | x |
| integrin alpha 6 | x | x |
| integrin alpha 6 | x | x |
| integrin beta 1 | x | x |
| integrin beta 2 (CD18) | x | x |
| integrin beta 3 | x | x |
| macrophage mannose receptor | x | x |
| MUC18/L-gicerin | x | x |
| notch 3 | x | x |
| protein tyrosine kinase, EGF receptor | x | x |
| protein tyrosine phosphatase, | x | x |
| receptor type mu | | |

TABLE 2
Common (T + N) Transmembrane Proteins (28/41)
Oct. 02, 2003

| | | | |
|---|---|---|---|
| acession | description | mass | TM domains |

TRANSPORTERS AND CHANNELS

| | | | |
|---|---|---|---|
| AAD10168 | calcium channel Trp4, putative capacitative | 103,051 | 8 |
| O35379 | multidrug resistance protein ABCC1 | 172,520 | 16 |

ADHESION MOLECULES

| | | | |
|---|---|---|---|
| CAA58782 | cadherin-VE | 88,138 | 2 |
| AAA62450 | calnexin | 65,360 | 1 |
| S44142 | integrin alpha 2 (α2β1) | 130,088 | 1 |
| I55534 | integrin alpha 3 (α3β1) | 117,964 | 2 |
| S44250 | integrin alpha 5 (α5β1) | 116,217 | 1 |
| CAA49527 | integrin alpha 6 (subunit for β1, β4) | 120,814 | 1 |
| S00551 | integrin alpha M (CD11b, Mac-1 alpha subunit for CD18) | 128,642 | 1 |
| CAA33272 | integrin beta 1 (subunit for α1-9, αV) | 91,501 | 1 |
| S04847 | integrin beta 2 (CD18; subunit for CD11a, CD11b, CD11c) | 88,144 | 1 |
| PN0510 | integrin beta 3 (subunit for αv, αIIb) | | 1 |
| Q9EPPI | L-glcerinMUC18 | 72,498 | 1 |

RECEPTORS, LIGANDS, KINASES AND PHOSPHATASES

| | | | |
|---|---|---|---|
| S25111 | alpha-2-macroglobulin receptor | 504,781 | 1 |
| CAA42219 | BGF receptor fragment | | 1 |
| AAA92709 | Fc gamma receptor IIb2 | 28,829 | 1 |
| BAA01065 | hepatocyte growth factor | 84,734 | 1 |
| ACM SIT | inositol 1,4,5-trisphosphate receptor | 316,680 | 6 |
| JE0273 | low density lipoprotein receptor-related protein 6 | 183,331 | 1 |
| A55840 | macrophage bacteria-binding receptor MARCO | 53,252 | 1 |
| I48922 | mannose 6-phosphate/insulin-like growth factor II receptor, cation-independent | 280,912 | 1 |
| A48925 | mannose receptor precursor, macrophage | 167,826 | 1 |
| S45306 | notch 3 | 257,655 | 1 |
| JC4976 | plexin 3 (semaphorin receptor family) | 211,540 | 1 |
| S12792 | protein tyrosine kinase (EC 2.7.1.112); Ltk (leukocyte tyrosine kinase) | 63,874 | 1 |
| A48066 | protein tyrosine phosphatase (EC 3.1.3.48), receptor type kappa | 166,217 | 2 |
| S17670 | protein tyrosine phosphatase (EC 3.1.3.48), receptor type mu | 165,394 | 1 |
| A56487 | signal recognition particle receptor beta chain | 29,579 | 1 |

TABLE 3a
HT29 Tumor-Specific (T − N) Transmembrane Proteins (32/160)
Oct. 02, 2003

| | | | |
|---|---|---|---|
| acession | description | mass | TM domains |

TRANSPORTERS AND CHANNELS

| | | | |
|---|---|---|---|
| Q9R183 | amino acid transporter B0+ | 72,552 | 12 |
| AAC52940 | calcium channel alpha 1 A, voltage-sensitive | 248,547 | 17 |
| AAD42069 | calcium channel, receptor-activated | 100,229 | 5 |
| S55473 | chloride channel 3 | 85,520 | 11 |
| AAD28473 | chloride channel 5 | 84,088 | 12 |
| AAD50604 | chloride channel CLC-2 | 100,366 | 11 |
| AAC17702 | chloride channel CLC6, putative | 98,431 | 7 |
| Q9JJZ9 | cyclic nucleotide-gated channel subunit CNG6 | 80,361 | 4 |
| BAA02254 | glutamate receptor channel subunit epsilon 4 | 144,301 | 4 |
| Q9ES78 | intestinal peptide transporter pept1 | 78,276 | 11 |
| JC4139 | potassium channel 1, G-protein-activated | 57,038 | 3 |
| S48739 | potassium channel, G protein-activated | 42,991 | 2 |
| AAB60688 | potassium channel, voltage-gated | 74,2986 | 3 |
| Q61923 | potassium channel, voltage-gated, shaker-related, member 6 | 59,138 | 4 |
| AAH09652 | sodium channel type I beta polypeptide, voltage-gated | 24,998 | 1 |
| AAH5742 | solute carrier family 24 protein (Slc24a3) | 36,354 | 5 |

ADHESION MOLECULES

| | | | |
|---|---|---|---|
| AAC06342 | activated leukocyte cell adhesion molecule CD166; ALCAM | 65,858 | 1 |
| CAA64857 | cadherin 8 | 88,548 | 1 |
| Q91Y21 | cadherin, protocadherin alpha 1 | 103,098 | 1 |
| Q91VD6 | cadherin, protocadherin beta 17 | 88,208 | 1 |
| Q91XZ9 | cadherin, protocadherin beta 20 | 88,265 | 1 |
| Q91XX8 | cadherin, protocadherin gamma B1 | 99,505 | 1 |
| Q91XW9 | cadherin, protocadherin gamma C5 | 102,645 | 1 |
| IJM SCN | cadherin-N precursor, neuronal | 100,226 | 1 |
| A53584 | cadherin-OB | 88319 | 1 |
| Q99KH1 | cadherin-related, hypothetical 67.8 kDa protein | 68,692 | 1 |
| Q9ERC8 | Down Syndrome Cell Adhesion Molecule | 224,068 | 1 |
| T10050 | integrin alpha v (subunit for β2, β3, β5, β6, β8) | 116,381 | 1 |
| ITB5_MOUSE | Integrin beta 5 (αVβ5) | 91,275 | 1 |
| Q9BQ17 | protein tyrosine phosphatase LAR (Leukocyte Adhesion Receptor) | 212,611 | 1 |
| Q9ESS7 | S-gicerin/MUC18 | 67,854 | 1 |
| T42215 | zonadhesin | 610,266 | 1 |

TABLE 3b
HT29 Tumor-Specific (T − N) Transmembrane Proteins (42/160)
Oct. 02, 2003
RECEPTORS, LIGANDS, KINASES AND PHOSPHATASES

| acession | description | mass | TM domains |
|---|---|---|---|
| AAD28372 | calcium-sensing receptor | 113,790 | 7 |
| Q99MX6 | CD163, hemoglobin scavenger receptor, macrophage | 125,242 | 2 |
| AAB00092 | CD55, decay-accelerating factor transmembrane | 45,456 | 2 |
| O88971 | CDO protein (Robo-related) | 136,930 | 1 |
| JC5599 | cholecystokinin-A receptor | 49,249 | 7 |
| S541B1 | BrbB3 binding protein EBP1 | 38,262 | 1 |
| S52417 | E-selectin ligand-1 | 137,680 | 1 |
| AAH16256 | Frizzled 4 | 61,328 | 7 |
| Q91ZB5 | G protein-coupled receptor (nociceptive somatosensory neurons) | 32,898 | 7 |
| S53530 | gamma-aminobutyric acid receptor beta 1 chain | 64,891 | 4 |
| JH0589 | glutamate receptor gamma-2 chain | 110,423 | 3 |
| S12874 | glutamate receptor GluR1 | 102,382 | 3 |
| S68700 | HPTP beta-like tyrosine phosphatase | 137,827 | 1 |
| Q9CU00 | ICAM-4 soluble | 20,956 | 1 |
| A48805 | IGF-1 receptor alpha subunit | 38,201 | 1 |
| S12357 | interleukin-6 receptor | 47,627 | 1 |
| Q9JIA8 | kalnate receptor GLUR7 3 subunit | 58,417 | 3 |
| JX0312 | LIF receptor alpha chain | 123,909 | 1 |
| O89821 | LIF receptor beta chain | 111,347 | 1 |
| Q91ZX7 | lipoprotein receptor-related protein | 624,010 | 1 |
| ML1X_MOUSE | melatonin-related receptor HB | 66,346 | 7 |
| AAA37168 | muscarinic acetylcholine receptor | 62,199 | 6 |
| BAA13050 | neuropeptide Y-Y2 receptor | 43,564 | 7 |
| A49542 | N-formyl peptide chemotactic receptor | 40,907 | 7 |
| AAA39803 | nicotinic acetylcholine receptor delta1 subunit | 69,564 | 4 |
| O35202 | pheromone receptor, putative | 94,016 | 7 |
| AAA37286 | protein tyrosine kinase; Bek FGF receptor | 93,087 | 1 |
| I58411 | protein tyrosine kinase; Brt (Tyro-3, Sky, Rse. EC 2.7.1.112) | 94,975 | 1 |
| S28926 | protein tyrosine kinase; Ret | 125,763 | 1 |
| I48310 | protein tyrosine kinase-related protein Ros | 264,521 | 1 |
| O35584 | protein tyrosine phosphatase; receptor-type | 163,181 | 2 |
| Q99LE2 | rhodopsin-like receptor, hypothetical protein | 37,022 | 6 |
| BAA76294 | semaphorin Y (sema6C) | 100,873 | 1 |
| Q9CZT5 | Silt-Like 2 | 73,488 | 1 |
| JC6539 | Smoothened | 88,809 | 7 |
| AAC16739 | sortilin-related receptor (gp250; LR11) | 232,879 | 1 |
| Q99MNB | sphingosine-1-phosphate receptor LPB4 | 41,824 | 7 |
| RWMSBC | T-cell receptor beta chain precursor (F5) | 34,644 | 1 |
| Q921T1 | TGF-beta 2, simlar to | 29,784 | 1 |
| Q9QYM9 | TMBFF2 (TM protein with EGF-like and 2 follistatin-like domains 2) | 43,389 | 2 |
| Q91ZV8 | Tumor Endothelial Marker 5 (7 TM; class II GPCR) | 144,747 | 6 |
| AAA40465 | tumor necrosis factor receptor | 61,772 | 2 |

TABLE 4a
Murine GSIB₄ Binding Proteins Identified in HT29
Tumor Xenograft But Not Normal Mouse Lung Samples

| | Human homologs with little published |
|---|---|
| murine protein | expression data in somatic tissues 19 |
| accession # | murine GSIB₄ binding protein |
| AAF71993 | ADAM-28 |
| CAA64857 | cadherin 8 |
| A53584 | cadherin OB |
| Q91Y21 | cadherin, protocadherin alpha 1 |
| Q91VD8 | cadherin, protocadherin beta 17 |
| Q91XZ9 | cadherin, protocadherin beta 20 |
| Q91XX8 | cadherin, protocadherin gamma B1 |
| Q91XW9 | cadherin, protocadherin gamma C5 |
| O88971 | CDO (Robo-related) |
| Q9ERC8 | Down Syndrome Cell Adhesion Molecule |
| S54181 | ErbB3 binding protein EBP1 |
| Q9D3K0 | LIB (LRRC15) |
| Q99KT6 | LRR and Ig domain containing hypothetical |
| | 53.5 kDa fragment |
| P97860 | LRRN3 (Ig and FN3 domain containing fragment) |
| T34101 | neural adhesion molecule p84 |
| Q9CZT5 | slit-like 2 |
| AAC16739 | sortilin-related receptor (LR11) |
| Q9QYM9 | TMEFF2 (TM protein with EGF-like and |
| | 2 follistatin-like domains 2) |
| T42215 | zonadhesin |

TABLE 4b
Human homologs known to be expressed by vasculature 42

| accession | GSIB₄ binding protein |
|---|---|
| | angiotensin converting factor (peptidyl |
| | dipeptidase A) |
| o89103 | C1Q receptor |
| IJMSCN | cadherin-N |
| | cadherin-VE |
| | calnexin |
| aab19065, | CD13 (aminopeptidase N) |
| AMPN_MOUSE | |
| Q99MX8 | CD163 (macrophage hemoglobin scavenger receptor) |
| AAC06342 | CD166 (activated leukocyte cell adhesion molecule) |
| AAB00092 | CD55 (decay accelerating factor) |
| | CD91 (alpha-2-macroglobulin receptor) |
| JC5599 | cholecystokinin A receptor |
| S51193 | epimorphin |
| | Fc gamma RIIb |
| AAH15256 | frizzled 4 |
| A48805 | IGF-1 receptor alpha subunit |
| | integrin alpha 2 |
| | integrin alpha 3 |
| | integrin alpha 5 |
| | integrin alpha 6 |
| T10050 | integrin alpha v |

TABLE 4C

| | |
|---|---|
| | integrin beta 1 |
| | integrin beta 2 (CD18) |
| | integrin beta 3 |
| ITB5_MOUSE | integrin beta 5 |
| JX0312 | LIF receptor alpha chain |
| O88821 | LIF receptor beta chain |
| | macrophage mannose receptor |
| | MUC18/L-gicerin |
| Q9ESS7 | MUC18/S-gicerin |
| BAA13050 | neuropeptide Y-Y2 receptor |
| A49542 | N-formyl peptide chemotactic receptor |
| | notch 3 |
| Q9ES83 | popeye protein 1 |
| I58411 | protein tyrosine kinase Brt (Tyro-3, Sky, Rse) |
| AAA37286 | protein tyrosine kinase, Bek |
| | protein tyrosine kinase, EGF receptor |
| | protein tyrosine phosphatase, receptor type mu |
| BAA76294 | semaphorin Y (6C) |
| JC5539 | smoothened |
| Q99MN8 | sphingosine-1-P receptor LPB4 |
| | (endothelial differentiation) |
| AAA40465 | TNF receptor |
| Q91ZV8 | tumor endothelial marker 5 |

1-168. (canceled)

169. A method for treating a hyperproliferative disease or disorder in an animal, comprising administering to an animal in need of treatment a composition comprising:
a) a binding molecule which specifically binds to a gene product selected from the group consisting of:
i) an LRRC15 polypeptide consisting of amino acids 1 to 581 of SEQ ID NO:2 or a fragment thereof,
ii) an LRRC15 variant polypeptide at least 70% identical to an LRRC15 polypeptide fragment consisting essentially of amino acids 22 to 581 of SEQ ID NO:2,
iii) an LRRC15 variant polypeptide at least 70% identical to an LRRC15 polypeptide fragment consisting essentially of amino acids 22 to 537 of SEQ ID NO:2,
iv) an LRRC15 variant polypeptide at least 70% identical to an LRRC15 polypeptide fragment consisting essentially of amino acids 22 to 538 of SEQ ID NO:2, and
v) an LRRC15 messenger RNA consisting essentially of nucleotides 1 to 5938 of SEQ ID NO:5 or a fragment thereof; and
b) a pharmaceutically acceptable carrier.

170. The method of claim 169, wherein said fragment consists essentially of amino acids 1 to 538 of SEQ ID NO:2.

171. The method of claim 169, wherein said fragment consists essentially of amino acids 1 to 537 of SEQ ID NO:2.

172. The method of claim 169, wherein said fragment consists essentially of amino acids 22 to 581 of SEQ ID NO:2.

173. The method of claim 169, wherein said fragment consists essentially of amino acids 22 to 538 of SEQ ID NO:2.

174. The method of claim 169, wherein said fragment consists essentially of amino acids 22 to 537 of SEQ ID NO:2.

175. The method of claim 169, wherein said fragment consists essentially of amino acids 127 to 157 of SEQ ID NO:2.

176. The method of claim 169, wherein said fragment consists essentially of amino acids 480 to 509 of SEQ ID NO:2.

177. The method of claim 169, wherein said fragment comprises one or more leucine-rich-repeat (LRR) regions selected from the group consisting of:
amino acids 51 to 75 of SEQ ID NO:2 (LRR1); amino acids 76 to 99 of SEQ ID NO:2 (LRR2); amino acids 100 to 123 of SEQ ID NO:2 (LRR3); amino acids 125 to 147 of SEQ ID NO:2 (LRR4); amino acids 148 to 171 of SEQ ID NO:2 (LRR5); amino acids 173 to 195 of SEQ ID NO:2 (LRR6); amino acids 196 to 219 of SEQ ID NO:2 (LRR7); amino acids 221 to 243 of SEQ ID NO:2 (LRR8); amino acids 244 to 267 of SEQ ID NO:2 (LRR9); amino acids 269 to 291 of SEQ ID NO:2 (LRR10); amino acids 292 to 315 of SEQ ID NO:2 (LRR11); amino acids 317 to 339 of SEQ ID NO:2 (LRR12); amino acids 340 to 363 of SEQ ID NO:2 (LRR13); amino acids 364 to 387 of SEQ ID NO:2 (LRR14); and amino acids 389 to 411 of SEQ ID NO:2 (LRR15); or one or more fragments selected from the group consisting of: amino acid 25 to amino acid 60 of SEQ ID NO:2; amino acid 85 to amino acid 95 of SEQ ID NO:2; amino acid 140 to amino acid 170 of SEQ ID NO:2; amino acid 200 to amino acid 215 of SEQ ID NO:2; amino acid 240 to amino acid 255 of SEQ ID NO:2; amino acid 365 to amino acid 380 of SEQ ID NO:2; amino acid 420 to amino acid 430 of SEQ ID NO:2; amino acid 480 to amino acid 505 of SEQ ID NO:2; amino acid 25 to amino acid 50 of SEQ ID NO:2; amino acid 75 to amino acid 115 of SEQ ID NO:2; from amino acid 145 to amino acid 175 of SEQ ID NO:2; from about amino acid 195 to about amino acid 215 of SEQ ID NO:2; from amino acid 225 to amino acid 270 of SEQ ID NO:2; about amino acid 285 to amino acid 325 of SEQ ID NO:2; from amino acid 326 to amino acid 385 of SEQ ID NO:2; from amino acid 386 to amino acid 450 of SEQ ID NO:2; and from amino acid 475 to amino acid 540 of SEQ ID NO:2.

178. The method of claim 169, wherein said binding molecule is selected from the group consisting of a fusion protein, an agent which elicits a T-cell response, a ligand, an antibody or immunospecific fragment thereof, and a small molecule.

179. The method of claim 169, wherein said gene product is an LRRC15 messenger RNA.

180. The method of claim 179, wherein said binding molecule is selected from the group consisting of an antisense oligonucleotide, an siRNA, a ribozyme, and a small molecule.

181. The method of claim 178, wherein said binding molecule is an antibody or immunospecific fragment thereof.

182. The method of any one of claims 181, wherein said antibody or fragment thereof is monoclonal.

183. The method of any one of claims 181, wherein said antibody or fragment thereof is humanized.

184. The method of any one of claims 181, wherein said antibody or fragment thereof is chimeric.

185. The method of any one of claims 181, wherein said antibody or fragment thereof is primatized.

186. The method of claim 169, wherein said binding molecule is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, and polyethylene glycol (PEG).

187. The method of claim 186, wherein said cytotoxic agent is selected from the group consisting of a radionuclide, a biotoxin, an enzymatically active toxin, a cytostatic or cytotoxic therapeutic agent, a prodrugs, an immunologically active ligand, and a biological response modifier.

188. The method of claim 186, wherein said detectable label is selected from the group consisting of an enzyme, a fluorescent label, a chemiluminescent label, a bioluminescent label, and a radioactive label.

189. The method of claim 169, wherein said hyperproliferative disease or disorder is selected from the group consisting of cancer, a neoplasm, a tumor, a malignancy, or a metastasis thereof.

190. The method of claim 189, wherein tumor cell proliferation is inhibited through the prevention or retardation of tumor angiogenesis.

191. The method of claim 189, wherein tumor cell proliferation is inhibited through the prevention or retardation of tumor spread to adjacent tissues.

192. The method of claim 189, wherein said hyperproliferative disease or disorder is a neoplasm located in the: prostate, colon, abdomen, bone, breast, digestive system, liver, pancreas, peritoneum, adrenal gland, parathyroid gland, pituitary gland, testicles, ovary, thymus, thyroid, eye, head, neck, central nervous system, peripheral nervous system, lymphatic system, pelvis, skin, soft tissue, spleen, thoracic region, or urogenital tract.

193. The method of claim 189, wherein said hyperproliferative disease is cancer, said cancer selected from the group consisting of: epithelial squamous cell cancer, melanoma, leukemia, myeloma, lung cancer, pancreatic cancer, cervical cancer, ovarian cancer, liver cancer, bladder cancer, breast cancer, colon cancer, renal cancer, prostate cancer, testicular cancer, thyroid cancer, and head and neck cancer.

194. The method of claim 193, wherein said cancer is selected from the group consisting of colon cancer, lung cancer, pancreatic cancer, ovarian cancer, and breast cancer.

195. The method of claim 169, wherein said animal is a mammal.

196. The method of claim 195, wherein said mammal is a human.

197. A method of detecting abnormal hyperproliferative cell growth in a patient, comprising:
(a) obtaining a biological sample from said patient;
(b) contacting said sample with a binding molecule which specifically binds to an LRRC15 gene product selected from the group consisting of:
(i) an LRRC15 polypeptide consisting of amino acids 1 to 581 of SEQ ID NO:2 or a fragment thereof,
(ii) an LRRC15 variant polypeptide at least 70% identical to an LRRC15 polypeptide fragment consisting essentially of amino acids 22 to 581 of SEQ ID NO:2,
(iii) an LRRC15 variant polypeptide at least 70% identical to an LRRC15 polypeptide fragment consisting essentially of amino acids 22 to 537 of SEQ ID NO:2,
(iv) an LRRC15 variant polypeptide at least 70% identical to an LRRC15 polypeptide fragment consisting essentially of amino acids 22 to 538 of SEQ ID NO:2, and
(v) an LRRC15 messenger RNA consisting essentially of nucleotides 1 to 5938 of SEQ ID NO:5 or a fragment thereof; and
(c) assaying the expression level of said LRRC15 gene product in said sample.

198. The method of claim 197, wherein said binding molecule is detectably labeled.

199. The method of claim 198, wherein said label is selected from the group consisting of: an enzyme, a fluorescent label, a luminescent label, a bioluminescent label, and a radioactive label.

200. The method of claim 197, wherein said binding molecule is selected from the group consisting of a fusion protein, an agent which elicits a T-cell response, a ligand, an antibody or immunospecific fragment thereof, and a small molecule.

201. The method of claim 197, wherein said gene product is an LRRC15 messenger RNA.

202. The method of claim 201, wherein said binding molecule is selected from the group consisting of an antisense oligonucleotide, an siRNA, a ribozyme, and a small molecule.

203. The method of claim 200, wherein said binding molecule is an antibody or immunospecific fragment thereof.

204. The method of claim 203, wherein said antibody or fragment thereof is monoclonal.

205. The method of claim 203, wherein said antibody or fragment thereof is humanized.

206. The method of claim 203, wherein said antibody or fragment thereof is chimeric.

207. The method of claim 203, wherein said antibody or fragment thereof is primatized.

208. A method of diagnosing a hyperproliferative disease or disorder in a patient, comprising:
(a) administering to said patient a sufficient amount of a detectably labeled binding molecule which specifically binds to an LRRC15 gene product selected from the group consisting of:
(i) an LRRC15 polypeptide consisting of amino acids 1 to 581 of SEQ ID NO:2 or a fragment thereof,
(ii) an LRRC15 variant polypeptide at least 70% identical to an LRRC15 polypeptide fragment consisting essentially of amino acids 22 to 581 of SEQ ID NO:2,
(iii) an LRRC15 variant polypeptide at least 70% identical to an LRRC15 polypeptide fragment consisting essentially of amino acids 22 to 537 of SEQ ID NO:2,
(iv) an LRRC15 variant polypeptide at least 70% identical to an LRRC15 polypeptide fragment consisting essentially of amino acids 22 to 538 of SEQ ID NO:2, and
(v) an LRRC15 messenger RNA consisting essentially of nucleotides 1 to 5938 of SEQ ID NO:5 or a fragment thereof;
(b) waiting for a time interval following said administration to allow said binding molecule to contact said gene product; and
(c) detecting the amount of said binding molecule bound to said gene product in said patient.

209. The method of claim 208, wherein said binding molecule comprises a label selected from the group consisting of: an enzyme, a fluorescent label, a luminescent label, a bioluminescent label, a radioactive label, a positron emitting metal, and a nonradioactive paramagnetic metal ion.

210. The method of claim 209, wherein said labeled binding molecule is detected in said patient by a method selected from the group consisting of: computed tomography (CT), position emission tomography (PET), magnetic resonance imaging (MRI), sonography, nuclear magnetic resonance imaging (NMR), electron spin resonance imaging (ESR), detection with a radiaion responsive surgical instrument, and detection with a fluorescence responsive scanning instrument.

211. The method of claim 208, wherein said labeled binding molecule preferentially accumulates at the location of cells which express LRRC15.

212. The method of claim 209, wherein said binding molecule is selected from the group consisting of a fusion protein, an agent which elicits a T-cell response, a ligand, an antibody or immunospecific fragment thereof, and a small molecule.

213. The method of claim 208, wherein said gene product is an LRRC15 messenger RNA.

214. The method of claim 213, wherein said binding molecule is selected from the group consisting of an antisense oligonucleotide, an siRNA, a ribozyme, and a small molecule.

215. The method of claim 212, wherein said binding molecule is an antibody or immunospecific fragment thereof.

216. The method of claim 215, wherein said antibody or fragment thereof is monoclonal.

217. The method of claim 215, wherein said antibody or fragment thereof is humanized.

218. The method of claim 215, wherein said antibody or fragment thereof is chimeric.

219. The method of claim 215, wherein said antibody or fragment thereof is primatized.
